(19) United States
(12) Patent Application Publication
(10) Pub. No.: US 2026/0124287 A1
(43) Pub. Date: May 7, 2026

(54) NEISSERIA MENINGITIDIS COMPOSITIONS AND METHODS THEREOF

(71) Applicant: Pfizer Inc., New York, NY (US)

(72) Inventors: Annaliesa Sybil Anderson, Upper Saddle River, NJ (US); John Lance Perez, Doylestown, PA (US); Kathrin Ute Jansen, New York, NY (US); Paul Liberator, Holmdel, NJ (US); Cuiwen Tan, Ridgewood, NJ (US); Thomas Richard Jones, Bluffton, SC (US); Johannes Frederik Beeslaar, Sandwich, Kent (GB); Judith Absalon, Bronx, NY (US); Jason Douglas Maguire, Chesapeake, VA (US); Shannon Lea Harris, Boston, MA (US)

(21) Appl. No.: 19/257,652

(22) Filed: Jul. 2, 2025

Related U.S. Application Data
(63) Continuation of application No. 17/764,153, now Pat. No. 12,383,611.
(60) Provisional application No. 62/907,097, filed on Sep. 27, 2019.
(60) Provisional application No. 63/040,498, filed on Jun. 17, 2020.

(51) Int. Cl. A61K 39/095; A61K 39/00; A61K 47/02; A61K 47/64; A61P 31/04; C07K 14/22

(52) U.S. Cl. CPC A61K 39/095; A61K 47/02; A61K 47/646; A61P 31/04; C07K 14/22; A61K 2039/545; A61K 2039/55505; A61K 2039/627; A61K 2039/70

(57) ABSTRACT

In one aspect, the disclosure relates to a composition including a factor H binding protein (fHBP) and a Neisseria meningitidis non-serogroup B capsular polysaccharide, and methods of use thereof. The disclosure further relates to uses of a composition that includes fHBP, such as, for example, uses to elicit an immune response against N. meningitidis serogroup B strains and non-serogroup B strains. The compositions and methods described herein are directed to administration in humans, including adults, adolescents, toddlers, and infants.

### CROSS REFERENCE TO RELATED APPLICATIONS

The present application claims the benefit of U.S. Provisional Patent Application 62/907,097, filed Sep. 27, 2019, and U.S. Provisional Patent Application 63/040,498, filed Jun. 17, 2020. All of the foregoing applications are hereby incorporated by reference in their entireties.

### REFERENCE TO SEQUENCE LISTING

This application is being filed electronically via EFS-Web and includes an electronically submitted sequence listing in .xml format. The .xml file contains a sequence listing entitled “PC072522B Sequence Listing.xml” created on Jun. 24, 2025 and having a size of 102,400 bytes. The sequence listing contained in this .xml file is part of the specification and is incorporated herein by reference in its entirety.

### FIELD OF THE DISCLOSURE

The present disclosure relates to Neisseria meningitidis compositions and methods thereof.

### BACKGROUND OF THE DISCLOSURE

Neisseria meningitidis is a Gram-negative encapsulated bacterium that can cause sepsis, meningitis, and death. N. meningitidis can be classified into at least 12 serogroups (including serogroups A, B, C, 29E, H, I, K, L, W-135 (mostly now referred to as W), X, Y and Z) based on chemically and antigenically distinctive polysaccharide capsules. Strains with five of the serogroups (A, B, C, Y, and W135) are responsible for the majority of disease.

Meningococcal meningitis is a devastating disease that can kill children and young adults within hours despite the availability of antibiotics. There is a need for improved immunogenic compositions against meningococcal serogroups A, B, C, Y, and W135 and/or X.

Currently, a cross-protective vaccine or composition effective against a wide range of MnB and meningococcal serogroups A, C, Y, and W and/or X isolates is not yet commercially available. Accordingly, a cross-protective vaccine or composition effective against diverse MnB and meningococcal serogroups A, C, Y, and W and/or X isolates is needed.

It is a further object of the disclosure to provide improved schedules for administering a meningococcal vaccine. Under the current recommendation scheme, there are four to five vaccinations given against meningococcal serogroups A, C, W, Y and B, given at different ages. There is an unmet need for efficient vaccinations that may to simplify immunization schedules and improve vaccination coverage to achieve further reductions in invasive meningococcal disease (IMD).

### SUMMARY OF THE DISCLOSURE

To meet these and other needs, the present disclosure relates to Neisseria meningitidis compositions and methods thereof.

The inventors discovered a method of inducing an immune response in a human, including administering to the human a composition comprising a) a polypeptide derived from a Neisseria meningitidis factor H binding protein (fHBP); (b) a Neisseria meningitidis serogroup A capsular saccharide conjugate; (c) a Neisseria meningitidis serogroup C capsular saccharide conjugate; (d) a Neisseria meningitidis serogroup W capsular saccharide conjugate; and (e) a Neisseria meningitidis serogroup Y capsular saccharide conjugate, wherein the composition induces an immune response to at least one of N. meningitidis serogroups A, C, W-135 and Y capsular polysaccharides and N. meningitidis serogroup B, wherein the immune response includes a titer of serum bactericidal antibodies that is higher than a titer of serum bactericidal antibodies induced by a respective licensed vaccine against the serogroup.

The inventors surprisingly discovered a method of inducing an immune response in a human, including administering to the human a composition comprising a) a first polypeptide derived from a Neisseria meningitidis factor H binding protein (fHBP); (b) a second polypeptide derived from a Neisseria meningitidis factor H binding protein (fHBP); (c) a Neisseria meningitidis serogroup A capsular saccharide conjugate; (d) a Neisseria meningitidis serogroup C capsular saccharide conjugate; (e) a Neisseria meningitidis serogroup W capsular saccharide conjugate; and (f) a Neisseria meningitidis serogroup Y capsular saccharide conjugate, wherein the composition induces an immune response to at least one of N. meningitidis serogroups A, C, W-135 and Y capsular polysaccharides and N. meningitidis serogroup B, wherein the immune response includes a titer of serum bactericidal antibodies that is higher than a titer of serum bactericidal antibodies induced by a respective licensed vaccine against the serogroup.

In some embodiments, the polypeptide includes an amino acid sequence having at least 70% identity to any one amino acid sequence selected from SEQ ID NO: 1 to SEQ ID NO: 62.

In a preferred embodiment, the composition includes (a) a first polypeptide including the amino acid sequence set forth in SEQ ID NO: 1; (b) a second polypeptide including the amino acid sequence set forth in SEQ ID NO: 2; (c) a Neisseria meningitidis serogroup A capsular saccharide conjugated to an adipic acid dihydrazide (ADH) linker by 1-cyano-4-dimethylamino pyridinium tetrafluoroborate, wherein the linker is conjugated to tetanus toxoid by carbodiimide chemistry; (d) a Neisseria meningitidis serogroup C capsular saccharide conjugated to an ADH linker by 1-cyano-4-dimethylamino pyridinium tetrafluoroborate, wherein the linker is conjugated to tetanus toxoid by carbodiimide chemistry; (e) a Neisseria meningitidis serogroup W capsular saccharide directly conjugated to tetanus toxoid by 1-cyano-4-dimethylamino pyridinium tetrafluoroborate, in the absence of a linker; and (f) a Neisseria meningitidis serogroup Y capsular saccharide directly conjugated to tetanus toxoid by 1-cyano-4-dimethylamino pyridinium tetrafluoroborate, in the absence of a linker.

In some embodiments, the composition elicits an immune response to any one of N. meningitidis serogroups A, C, W-135 and Y, wherein said serum bactericidal antibody response is higher than that elicited by a licensed N. meningitidis serogroups A, C, W-135 and Y meningococcal capsular polysaccharide vaccine.

In some embodiments, the composition elicits an immune response to N. meningitidis serogroup A, wherein said serum bactericidal antibody response is higher than that elicited by a licensed N. meningitidis serogroups A, C, W-135 and Y meningococcal capsular polysaccharide vaccine.

In some embodiments, the composition elicits an immune response to N. meningitidis serogroup C, wherein said serum bactericidal antibody response is higher than that elicited by a licensed N. meningitidis serogroups A, C, W-135 and Y meningococcal capsular polysaccharide vaccine.

In some embodiments, the composition elicits an immune response to N. meningitidis serogroup W, wherein said serum bactericidal antibody response is higher than that elicited by a licensed N. meningitidis serogroups A, C, W-135 and Y meningococcal capsular polysaccharide vaccine.

In some embodiments, the composition elicits an immune response to N. meningitidis serogroup Y, wherein said serum bactericidal antibody response is higher than that elicited by a licensed N. meningitidis serogroups A, C, W-135 and Y meningococcal capsular polysaccharide vaccine.

In some embodiments, the composition elicits an immune response to each of the N. meningitidis serogroups A, C, W-135 and Y, wherein said serum bactericidal antibody response is higher than that elicited by a licensed N. meningitidis serogroups A, C, W-135 and Y meningococcal capsular polysaccharide vaccine.

In some embodiments, the composition elicits an immune response to N. meningitidis serogroup B, wherein said serum bactericidal antibody response is higher than that elicited by a licensed meningococcal serogroup B factor H binding vaccine.

In some embodiments, the composition elicits an immune response to each of the N. meningitidis serogroups A, C, W-135 and Y, wherein said serum bactericidal antibody response to each of the N. meningitidis serogroups A, C, W-135 and Y capsular polysaccharides is higher than that elicited by a licensed N. meningitidis serogroups A, C, W-135 and Y meningococcal capsular polysaccharide vaccine; and the composition elicits an immune response to N. meningitidis serogroup B, wherein said serum bactericidal antibody response is higher than that elicited by a licensed meningococcal serogroup B factor H binding vaccine; wherein the licensed N. meningitidis serogroups A, C, W-135 and Y meningococcal capsular polysaccharide vaccine and the licensed meningococcal serogroup B factor H binding vaccine are administered sequentially and are not in a combined dose.

In some embodiments, the composition includes an adjuvant. In some embodiments, the composition includes an aluminum adjuvant. In some embodiments, the composition includes aluminum hydroxide. In some embodiments, the composition includes aluminum phosphate. In some embodiments, the composition includes including aluminum.

In some embodiments, at least 90% of the first polypeptide is bound to aluminum in the composition. In some embodiments, at least 90% of the second polypeptide is bound to aluminum in the composition.

In some embodiments, the composition is formulated as a sterile liquid. In some embodiments, the composition includes a pharmaceutically acceptable preservative. In some embodiments, the composition includes polysorbate-80. In some embodiments, the composition includes Tris-HCl; sodium chloride; sucrose; histidine; polysorbate 80; and aluminum phosphate.

In some embodiments, the composition includes about 120 μg/ml of the first polypeptide; about 120 μg/ml of the second polypeptide; about 0.5 mg/ml aluminum as aluminum phosphate; about 0.02 mg polysorbate-80; about 10 mM histidine; and about 150 mM sodium chloride.

In some embodiments, the composition includes about 60 μg of the first polypeptide; about 60 μg of the second polypeptide; about 5 μg of the MenA capsular saccharide conjugated to about 7.5 μg TT; about 5 μg of the MenC capsular saccharide conjugated to about 7.5 μg TT; about 5 μg of the MenW capsular saccharide conjugated to about 3.75 μg TT; about 5 μg of the MenY capsular saccharide conjugated to about 3.25 μg TT; about 97 μg Tris-HCl, pH 6.8±0.3; 4.69-4.71 mg of sodium chloride; about 28 mg of sucrose; about 0.78 mg of L-Histidine; about 0.02 mg polysorbate-80; about 0.25 mg aluminum; and further including 0.5 mL water, per dose.

In some embodiments, the immune response includes a serum bactericidal antibody. In some embodiments, the composition is capable of eliciting a booster immune response to at least one of the N. meningitidis serogroups A, C, W-135 and Y. In some embodiments, the composition is capable of eliciting a booster immune response to N. meningitidis serogroup B.

In some embodiments, the immune response is elicited in a human up to 25 years old. In some embodiments, the immune response is elicited in a human aged at least 2 months to 25 years old. In some embodiments, the immune response is elicited in a human 10 to 25 years old. In some embodiments, the immune response is elicited in a human 10 to 26 years old. In some embodiments, the immune response is elicited in a human aged 12 to <18 Months or 18 to <24 Months. In some embodiments, the immune response is elicited in a human aged 18 to <24 Months. In some embodiments, the immune response is elicited in a human aged ≥24 Months to <10 Years.

In some embodiments, the immune response is elicited in a human that is seronegative against N. meningitidis serogroups A, C, W-135 and Y. In some embodiments, the immune response is elicited in a human that is seropositive against N. meningitidis serogroups A, C, W-135 and Y.

In some embodiments, the composition is administered to the human in at least two doses, wherein the second dose is about 6 months after the first dose. In some embodiments, the human is at least 10 years of age and at most 17 years of age. In some embodiments, a third dose of the composition is administered to the human, wherein the human is at least 16 years of age.

In some embodiments, the composition is administered to the human in at most two doses, wherein the second dose is about 6 months after the first dose.

In some embodiments, the composition elicits an immune response against A22. In some embodiments, the composition elicits an immune response against A56. In some embodiments, the composition elicits an immune response against B24. In some embodiments, the composition elicits an immune response against B44.

In some embodiments, the composition includes about 60 μg of the first polypeptide; about 60 μg of the second polypeptide; about 5 μg of the MenA capsular saccharide conjugated to about 7.5 μg TT; about 5 μg of the MenC capsular saccharide conjugated to about 7.5 μg TT; about 5 μg of the MenW capsular saccharide conjugated to about 3.75 μg TT; about 5 μg of the MenY capsular saccharide conjugated to about 3.25 μg TT; about 97 μg Tris-HCl, pH 6.8±0.3; 4.69-4.71 mg of sodium chloride; about 28 mg of sucrose; about 0.78 mg of L-Histidine; about 0.02 mg polysorbate-80; about 0.25 mg aluminum; and further including 0.5 mL water, per dose.

A composition including (a) a first polypeptide derived from a Neisseria meningitidis factor H binding protein (fHBP); (b) a second polypeptide derived from a Neisseria meningitidis factor H binding protein (fHBP); (c) a Neisseria meningitidis serogroup A capsular saccharide conjugate; (d) a Neisseria meningitidis serogroup C capsular saccharide conjugate; (e) a Neisseria meningitidis serogroup W capsular saccharide conjugate; and (f) a Neisseria meningitidis serogroup Y capsular saccharide conjugate; wherein the composition elicits an immune response to at least one of N. meningitidis serogroups A, C, W-135 and Y, wherein said serum bactericidal antibody response is higher than that elicited by a licensed vaccine against the N. meningitidis serogroup.

In some embodiments, the polypeptide includes an amino acid sequence having at least 70% identity to any one amino acid sequence selected from SEQ ID NO: 1 to SEQ ID NO: 62.

A composition including (a) a first polypeptide derived from a Neisseria meningitidis factor H binding protein (fHBP); (b) a second polypeptide derived from a Neisseria meningitidis factor H binding protein (fHBP); (c) a Neisseria meningitidis serogroup A capsular saccharide conjugated to an adipic acid dihydrazide (ADH) linker by 1-cyano-4-dimethylamino pyridinium tetrafluoroborate, wherein the linker is conjugated to tetanus toxoid by carbodiimide chemistry; (d) a Neisseria meningitidis serogroup C capsular saccharide conjugated to an ADH linker by 1-cyano-4-dimethylamino pyridinium tetrafluoroborate, wherein the linker is conjugated to tetanus toxoid by carbodiimide chemistry; (e) a Neisseria meningitidis serogroup W capsular saccharide directly conjugated to tetanus toxoid by 1-cyano-4-dimethylamino pyridinium tetrafluoroborate, in the absence of a linker; and (f) a Neisseria meningitidis serogroup Y capsular saccharide directly conjugated to tetanus toxoid by 1-cyano-4-dimethylamino pyridinium tetrafluoroborate, in the absence of a linker; wherein the composition elicits an immune response to at least one of the N. meningitidis serogroups A, C, W-135 and Y, wherein said serum bactericidal antibody response is higher than that elicited by a licensed N. meningitidis serogroups A, C, W-135 and Y meningococcal capsular polysaccharide vaccine.

A method for inducing an immune response against a Neisseria meningitidis serogroup B subfamily A strain and against a Neisseria meningitidis serogroup B subfamily B strain in human, including administering to the human an effective amount of the composition.

A method for inducing an immune response against a Neisseria meningitidis serogroup A, a Neisseria meningitidis serogroup C, a Neisseria meningitidis serogroup W, and/or a Neisseria meningitidis serogroup Y strain in a human, including administering to the human an effective amount of the composition.

A method for inducing an immune response against a Neisseria meningitidis serogroup A, Neisseria meningitidis serogroup B, a Neisseria meningitidis serogroup C, a Neisseria meningitidis serogroup W, and/or a Neisseria meningitidis serogroup Y strain in a human, including administering to the human an effective amount of the composition.

A method for inducing an immune response against a Neisseria meningitidis serogroup A, Neisseria meningitidis serogroup B, a Neisseria meningitidis serogroup C, a Neisseria meningitidis serogroup W, a Neisseria meningitidis serogroup Y strain, and/or a Neisseria meningitidis serogroup X strain in a human, including administering to the human an effective amount of the composition.

In some embodiments, the patient has not previously received a multivalent meningococcal capsular saccharide-carrier protein conjugate vaccine prior to the first administration of the composition.

In some embodiments, the patient previously received a multivalent meningococcal capsular saccharide-carrier protein conjugate vaccine prior to the first administration of the composition.

### BRIEF DESCRIPTION OF THE DRAWINGS

FIG. 1A-B—Immune Responses as Measured in hSBA to (FIG. 1A) MenB Test Strains at 1 Month After Dose 2 and (FIG. 1B) MenA, MenC, MenW, and MenY Test Strains at 1 Month After Doses 1 and 2. Error bars represent 95% Cis. MenB strains note FHbp variants in parentheses. *MenABCWY, n=418-4432; MenB-FHbp, n=814-850. ^{†}Composite response=hSBA titer ≥LLOQ for all 4 MenB test strains. *MenABCWY, n=227-262; MenACWY-CRM, n=446-506. § MenABCWY, n=187-257; MenACWY-CRM, n=370-495. hSBA=serum bactericidal activity with human complement; MenA=meningococcal serogroup A; LLOQ=lower limit of quantitation; m-month; MenABCWY=pentavalent serogroups A, B, C, W, Y vaccine; MenACWY-CRM=MENVEO®, meningococcal (groups A, C, Y, and W-135) oligosaccharide CRM197 conjugate vaccine (quadrivalent meningococcal CRM vaccine); MenB=meningococcal serogroup B; MenB-FHbp=TRUMENBA®, meningococcal group B bivalent recombinant lipoprotein 2086 (bivalent rLP2086). vaccine; MenC=meningococcal serogroup C; MenW=meningococcal serogroup W; MenY=meningococcal serogroup Y; PD=postdose.

FIG. 2A-B. (FIG. 2A) Local Reactions and (FIG. 2B) Systemic Events Reported Within 7 Days After Any Dose. MenABCWY, n=542; MenB-FHbp+MenACWY-CRM, n=1050. *At the MenABCWY/MenB-FHbp injection site. MenABCWY=pentavalent serogroups A, B, C, W, Y vaccine; MenACWY-CRM=MENVEO®, meningococcal (groups A, C, Y, and W-135) oligosaccharide CRM197 conjugate vaccine (quadrivalent meningococcal CRM vaccine); MenB-FHbp=TRUMENBA®, meningococcal group B bivalent recombinant lipoprotein 2086 (bivalent rLP2086).

FIG. 3. Estimated cases averted over 10 years under various vaccine administration strategies compared to a hypothetical no vaccination scenario.

### SEQUENCE IDENTIFIERS

SEQ ID NO: 1 sets forth the amino acid sequence for a recombinant N. meningitidis, serogroup B, 2086 variant A05 polypeptide antigen.

SEQ ID NO: 2 sets forth the amino acid sequence for a recombinant N. meningitidis, serogroup B, 2086 variant B01 polypeptide antigen.

SEQ ID NO: 3 sets forth the amino acid residues at positions 1-4 of SEQ ID NO: 1 and SEQ ID NO: 2.

SEQ ID NO: 4 sets forth the amino acid sequence of the N-terminus of a recombinant Neisserial Subfamily A LP2086 polypeptide (rLP2086) (A05) polypeptide.

SEQ ID NO: 5 sets forth the amino acid sequence of the N-terminus of Neisserial Subfamily A LP2086 M98250771 polypeptide (A05) polypeptide.

SEQ ID NO: 6 sets forth the amino acid sequence for N. meningitidis, serogroup B, 2086 variant B153.

SEQ ID NO: 7 sets forth the amino acid sequence for N. meningitidis, serogroup B, 2086 variant A04.

SEQ ID NO: 8 sets forth the amino acid sequence for N. meningitidis, serogroup B, 2086 variant A05 SEQ ID NO: 9 sets forth the amino acid sequence for N. meningitidis, serogroup B, 2086 variant A12.

SEQ ID NO: 10 sets forth the amino acid sequence for N. meningitidis, serogroup B, 2086 variant A22.

SEQ ID NO: 11 sets forth the amino acid sequence for N. meningitidis, serogroup B, 2086 variant B02.

SEQ ID NO: 12 sets forth the amino acid sequence for N. meningitidis, serogroup B, 2086 variant B03.

SEQ ID NO: 13 sets forth the amino acid sequence for N. meningitidis, serogroup B, 2086 variant B09.

SEQ ID NO: 14 sets forth the amino acid sequence for N. meningitidis, serogroup B, 2086 variant B22.

SEQ ID NO: 15 sets forth the amino acid sequence for N. meningitidis, serogroup B, 2086 variant B24.

SEQ ID NO: 16 sets forth the amino acid sequence for N. meningitidis, serogroup B, 2086 variant B44.

SEQ ID NO: 17 sets forth the amino acid sequence for N. meningitidis, serogroup B, 2086 variant B16.

SEQ ID NO: 18 sets forth the amino acid sequence for N. meningitidis, serogroup B, 2086 variant A07.

SEQ ID NO: 19 sets forth the amino acid sequence for N. meningitidis, serogroup B, 2086 variant A19.

SEQ ID NO: 20 sets forth the amino acid sequence for N. meningitidis, serogroup B, 2086 variant A06.

SEQ ID NO: 21 sets forth the amino acid sequence for N. meningitidis, serogroup B, 2086 variant A15.

SEQ ID NO: 22 sets forth the amino acid sequence for N. meningitidis, serogroup B, 2086 variant A29.

SEQ ID NO: 23 sets forth the amino acid sequence for N. meningitidis, serogroup B, 2086 variant B15.

SEQ ID NO: 24 sets forth the amino acid sequence of the N-terminus of a recombinant Neisserial Subfamily B LP2086 polypeptide (rLP2086) (B01) polypeptide.

SEQ ID NO: 25 sets forth the amino acid sequence of the N-terminus of Neisserial Subfamily B LP2086 CDC-1573 polypeptide (B01) polypeptide.

SEQ ID NO: 26 sets forth the amino acid sequence for N. meningitidis serogroup A strain expressing factor H binding protein (fHBP) B16.

SEQ ID NO: 27 sets forth the amino acid sequence for a N. meningitidis serogroup C strain expressing fHBP A10. SEQ ID NO: 27 also sets forth the amino acid sequence for a N. meningitidis serogroup W strain expressing fHBP A10.

SEQ ID NO: 28 sets forth the amino acid sequence for a N. meningitidis serogroup W strain expressing fHBP A19.

SEQ ID NO: 29 sets forth the amino acid sequence for a N. meningitidis serogroup Y strain expressing fHBP B47.

SEQ ID NO: 30 sets forth the amino acid sequence for a N. meningitidis serogroup X strain expressing fHBP B49.

SEQ ID NO: 31 sets forth the amino acid sequence for a N. meningitidis, serogroup B, 2086 variant B16.

SEQ ID NO: 32 sets forth the amino acid sequence for a N. meningitidis, serogroup B, 2086 variant A07.

SEQ ID NO: 33 sets forth the amino acid sequence for a N. meningitidis, serogroup B, 2086 variant A19.

SEQ ID NO: 34 sets forth the amino acid sequence for a N. meningitidis, serogroup B, 2086 variant A06.

SEQ ID NO: 35 sets forth the amino acid sequence for a N. meningitidis, serogroup B, 2086 variant A15.

SEQ ID NO: 36 sets forth the amino acid sequence for a N. meningitidis, serogroup B, 2086 variant A29.

SEQ ID NO: 37 sets forth the amino acid sequence for a N. meningitidis, serogroup B, 2086 variant B15.

SEQ ID NO: 38 sets forth the amino acid sequence for a N. meningitidis serogroup A strain expressing factor H binding protein (fHBP) B16.

SEQ ID NO: 39 sets forth the amino acid sequence for a N. meningitidis serogroup C strain expressing fHBP A10. SEQ ID NO: 39 also sets forth the amino acid sequence for a N. meningitidis serogroup W strain expressing fHBP A10.

SEQ ID NO: 40 sets forth the amino acid sequence for a N. meningitidis serogroup W strain expressing fHBP A19.

SEQ ID NO: 41 sets forth the amino acid sequence for a N. meningitidis serogroup Y strain expressing fHBP B47.

SEQ ID NO: 42 sets forth the amino acid sequence for a N. meningitidis serogroup X strain expressing fHBP B49.

SEQ ID NO: 43 sets forth the amino acid sequence for a N. meningitidis, serogroup B, 2086 variant B44.

SEQ ID NO: 44 sets forth the amino acid sequence for a N. meningitidis, serogroup B, 2086 variant B09.

SEQ ID NO: 45 sets forth the amino acid sequence for a N. meningitidis, serogroup B, 2086 variant B09.

SEQ ID NO: 46 sets forth the amino acid sequence for a N. meningitidis, serogroup B, 2086 variant A05.

SEQ ID NO: 47 sets forth the amino acid sequence for a N. meningitidis, serogroup B, 2086 variant B01.

SEQ ID NO: 48 sets forth the amino acid sequence for a N. meningitidis, serogroup B, 2086 variant B01, which includes an N-terminal Cys at amino acid position 1.

SEQ ID NO: 49 sets forth the amino acid sequence for a N. meningitidis, serogroup B, 2086 variant B15, which includes an N-terminal Cys at amino acid position 1.

SEQ ID NO: 50 sets forth the amino acid sequence for a N. meningitidis, serogroup B, 2086 variant B16, which includes an N-terminal Cys at amino acid position 1.

SEQ ID NO: 51 sets forth the amino acid sequence for a N. meningitidis, serogroup B, 2086 variant B22.

SEQ ID NO: 52 sets forth the amino acid sequence for a N. meningitidis, serogroup B, 2086 variant A22.

SEQ ID NO: 53 sets forth the amino acid sequence for a N. meningitidis, serogroup B, 2086 variant A12.

SEQ ID NO: 54 sets forth the amino acid sequence for a N. meningitidis, serogroup B, 2086 variant A22.

SEQ ID NO: 55 sets forth the amino acid sequence for a N. meningitidis serogroup B, 2086 variant A62, which includes an N-terminal Cys at amino acid position 1.

SEQ ID NO: 56 sets forth the amino acid sequence for a N. meningitidis, serogroup B, 2086 variant A62.

SEQ ID NO: 57 sets forth the amino acid sequence for a N. meningitidis, serogroup B, 2086 variant A29, which includes an N-terminal Cys at amino acid position 1.

SEQ ID NO: 58 sets forth the amino acid sequence for a N. meningitidis, serogroup B, 2086 variant B22.

SEQ ID NO: 59 sets forth the amino acid sequence for a N. meningitidis, serogroup B, 2086 variant A05.

SEQ ID NO: 60 sets forth the amino acid sequence for a N. meningitidis, serogroup B, 2086 variant A05.

SEQ ID NO: 61 sets forth the amino acid sequence for a N. meningitidis, serogroup B, 2086 variant B24.

SEQ ID NO: 62 sets forth the amino acid sequence for a N. meningitidis, serogroup B, 2086 variant B24.

SEQ ID NO: 63 sets forth the amino acid sequence for a N. meningitidis, serogroup B, 2086 variant A02

SEQ ID NO: 64 sets forth the amino acid sequence for a N. meningitidis, serogroup B, 2086 variant A28.

SEQ ID NO: 65 sets forth the amino acid sequence for a N. meningitidis, serogroup B, 2086 variant A42.

SEQ ID NO: 66 sets forth the amino acid sequence for a N. meningitidis, serogroup B, 2086 variant A63.

SEQ ID NO: 67 sets forth the amino acid sequence for a N. meningitidis, serogroup B, 2086 variant A76.

SEQ ID NO: 68 sets forth the amino acid sequence for a N. meningitidis, serogroup B, 2086 variant B05.

SEQ ID NO: 69 sets forth the amino acid sequence for a N. meningitidis, serogroup B, 2086 variant B07.

SEQ ID NO: 70 sets forth the amino acid sequence for a N. meningitidis, serogroup B, 2086 variant B08.

SEQ ID NO: 71 sets forth the amino acid sequence for a N. meningitidis, serogroup B, 2086 variant B13.

SEQ ID NO: 72 sets forth the amino acid sequence for a N. meningitidis, serogroup B, 2086 variant B52.

SEQ ID NO: 73 sets forth the amino acid sequence for a N. meningitidis, serogroup B, 2086 variant B107.

SEQ ID NO: 74 sets forth the amino acid sequence for a N. meningitidis, serogroup B, 2086 variant A56.

### DETAILED DESCRIPTION

The inventors discovered that a composition including at least one factor H binding protein (fHBP) and N. meningitidis serogroup A, C, W, and Y capsular saccharide conjugates is stable and immunogenic. The inventors further discovered that the composition induces an immune response in humans against at least 2, 3, 4, or 5 of N. meningitidis serogroups A, C, W, Y, and B. Surprisingly, the immune response elicited is higher than an immune response elicited by licensed vaccines against N. meningitidis.

In one aspect, the composition includes at least one polypeptide derived from a N. meningitidis factor H binding protein and at least one meningococcal capsular saccharide conjugated to a carrier protein.

Protein Derived from Factor H Binding Protein (fHBP).

In one embodiment, the composition includes any fHBP, such as, for example, any one of the following polypeptides: B24, B16, B44, A22, B03, B09, A12, A19, A05, A07, A06, A15, A29, B01, A62, B15, and any combination thereof. Preferably, the composition includes a combination of A05 and B01 polypeptides. In another preferred embodiment, the composition includes a combination of B24 and A05 polypeptides. In another embodiment, the composition includes a combination of A05, A12, B09, and B44 polypeptides. In one embodiment, the composition includes a lipidated fHBP. In one embodiment, the composition does not include a non-lipidated fHBP.

In another embodiment, the composition includes a non-lipidated fHBP, such as any one of the non-lipidated fHBP described in International Patent Publication No. WO2012/032489, US Patent Publication No. US20120093852, International Patent Publication No. WO2013/132452, and US Patent Publication No. US20160030543, which are each incorporated herein by reference in their entirety. In one embodiment, the composition includes at least one non-lipidated fHBP and at least one lipidated fHBP.

In some embodiments, the composition includes a polypeptide having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.9% identity to the amino acid sequence set forth in any one of SEQ ID NO: 1, SEQ ID NO: 2, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, and SEQ ID NO: 62.

In a preferred embodiment, the composition includes (a) a first polypeptide including the amino acid sequence set forth in SEQ ID NO: 1; (b) a second polypeptide including the amino acid sequence set forth in SEQ ID NO: 2; (c) a Neisseria meningitidis serogroup A capsular saccharide conjugated to an adipic acid dihydrazide (ADH) linker by 1-cyano-4-dimethylamino pyridinium tetrafluoroborate, wherein the linker is conjugated to tetanus toxoid by carbodiimide chemistry; (d) a N. meningitidis serogroup C capsular saccharide conjugated to an ADH linker by 1-cyano-4-dimethylamino pyridinium tetrafluoroborate, wherein the linker is conjugated to tetanus toxoid by carbodiimide chemistry; (e) a N. meningitidis serogroup W capsular saccharide directly conjugated to tetanus toxoid by 1-cyano-4-dimethylamino pyridinium tetrafluoroborate, in the absence of a linker; and (f) a N. meningitidis serogroup Y capsular saccharide directly conjugated to tetanus toxoid by 1-cyano-4-dimethylamino pyridinium tetrafluoroborate, in the absence of a linker.

### First Polypeptide; MNB RLP2086 Subfamily A (A05) Protein

In one embodiment, the composition includes a first polypeptide having the amino acid sequence set forth in SEQ ID NO: 1. The polypeptide is a modified factor H binding protein (fHBP) from N. meningitidis strain M98250771. A description of fHBP is disclosed in WO2012032489 and US patent publication US 2012/0093852, which are each incorporated by reference in their entirety. The polypeptide is N-terminally lipidated with three predominant fatty acids C16:0, C16:1, and C18:1 covalently linked at three positions of the polypeptide. The first polypeptide includes a total of 258 amino acids.

The representative primary structure of the MnB rLP2086 A05 protein is presented in FIG. 4 of U.S. Pat. No. 10,183,070. The primary structure of the protein is illustrated in FIG. 4 of U.S. Pat. No. 10,183,070 using a single letter notation for all amino acids except for the N-terminal cysteine and glyceryl moieties (illustrated using full chemical formula). This structure includes the primary structure of the protein sequence in which the N-terminal cysteine residue is lipidated. The amino group of the N-terminal cysteine residue at the protein N-terminus is attached to a fatty acid (R1) forming an amide linkage and the cysteinyl sulfhydryl group is attached to a glycerol moiety containing two ester-bound fatty acids (R2). The structure of R1 is deduced to be hexadecanoic acid (C16:0) and the structures of R2 vary depending on the MnB rLP2086 isoforms.

The first polypeptide includes two modifications introduced in the N-terminal region of the polypeptide, as compared to the corresponding wild-type sequence from N. meningitidis strain M98250771. A glycine in the second position is added as a consequence of introducing a cloning site. A second modification includes the deletion of four amino acids. Accordingly, in one embodiment, the first polypeptide includes a C-G-S-S sequence (SEQ ID NO: 3) at the N-terminus. See SEQ ID NO: 1, first four amino acid residues.

The N-terminal differences between the first polypeptide sequence and the wild-type Neisserial sequence is shown below. Accordingly, in one embodiment, the first polypeptide includes at least the first 2, 3, 4, 5, 6, 7, 8, 9, 10, 11, 12, 13, or more amino acid residues of the amino acid sequence set forth in SEQ ID NO: 1. Preferably, the first polypeptide includes at least the first 4, more preferably at least the first 6, and most preferably, at least the first 8 amino acid residues of SEQ ID NO: 1.

Comparison of Predicted N-Terminal Sequences of
Recombinant and Neisserial Subfamily A LP2086
Polypeptide

| | |
|---|---|
| rLP2086 M98250771 | CGSS-----GGGGVAAD |
| | (SEQ ID NO: 4) |
| Neisserial LP2086 | C-SSGS-GSGGGGVAAD |
| M98250771 | (SEQ ID NO: 5) |

>A05 (SEQ ID NO: 1)
CGSSGGGGVAADIGTGLADALTAPLDHKDKGLKSLTLEDSISQNGTLTLS
AQGAEKTFKVGDKDNSLNTGKLKNDKISRFDFVQKIEVDGQTITLASGEF
QIYKQDHSAVVALQIEKINNPDKIDSLINQRSFLVSGLGGEHTAFNQLPS
GKAEYHGKAFSSDDAGGKLTYTIDFAAKQGHGKIEHLKTPEQNVELASAE
LKADEKSHAVILGDTRYGSEEKGTYHLALFGDRAQEIAGSATVKIREKVH
EIGIAGKQ

In one embodiment, the first polypeptide includes the amino acid sequence set forth in SEQ ID NO: 1. In one embodiment, the first polypeptide has a total of 258 amino acids. In one embodiment, the first polypeptide does not include an amino acid sequence having less than 100% sequence identity to SEQ ID NO: 1. In another embodiment, the first polypeptide consists of the amino acid sequence set forth in SEQ ID NO: 1. In another embodiment, the first polypeptide includes the amino acid sequence KDN. See for example, amino acid residues 73-75 of SEQ ID NO: 1. In another embodiment, the first polypeptide includes the amino acid sequence set forth in SEQ ID NO: 3 at the N-terminus of the polypeptide. In another embodiment, the first polypeptide includes the amino acid sequence set forth in SEQ ID NO: 4 at the N-terminus of the polypeptide.

In a preferred embodiment, the first polypeptide is readily expressed in a recombinant host cell using standard techniques known in the art. In another preferred embodiment, the first polypeptide includes a bactericidal epitope on the N- and/or C-domain of SEQ ID NO: 1. In one embodiment, the first polypeptide includes at least the first 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues of the amino acid sequence set forth in SEQ ID NO: 1. Preferably, the first polypeptide includes at least the first 2, more preferably at least the first 4, and most preferably, at least the first 8 amino acid residues of SEQ ID NO: 1.

In another embodiment, the first polypeptide includes at least the last 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues of the amino acid sequence set forth in SEQ ID NO: 1.

In one embodiment, the composition includes about 30 μg/ml of a first polypeptide including the amino acid sequence set forth in SEQ ID NO: 1. In one preferred embodiment, the composition includes about 60 μg of a first polypeptide including the amino acid sequence set forth in SEQ ID NO: 1. In one preferred embodiment, the composition includes about 60 μg of a first polypeptide including the amino acid sequence set forth in SEQ ID NO: 1, wherein the composition preferably has a total volume of 0.5 ml. In another embodiment, the composition includes about 120 μg/ml of a first polypeptide including the amino acid sequence set forth in SEQ ID NO: 1.

### Second Polypeptide; MNB RLP2086 Subfamily B (B01) Protein

In one embodiment, the composition includes a second polypeptide having the amino acid sequence set forth in SEQ ID NO: 2. The polypeptide is a factor H binding protein (fHBP) from N. meningitidis strain CDC1573. A description of fHBP is disclosed in WO2012032489 and US patent publication US 2012/0093852, which are each incorporated by reference in their entirety. The polypeptide is N-terminally lipidated with three predominant fatty acids C16:0, C16:1, and C18:1 covalently linked at three positions of the polypeptide. The second polypeptide includes a total of 261 amino acids.

The representative primary structure of the MnB rLP2086 B01 protein is presented in FIG. 5 of U.S. Pat. No. 10,183,070. The primary structure of the protein is illustrated in FIG. 5 of U.S. Pat. No. 10,183,070 using a single letter notation for all amino acids except for the N-terminal cysteine and glyceryl moieties (illustrated using full chemical formula). This structure includes the primary structure of the protein sequence in which the N-terminal cysteine residue is lipidated. The amino group of the N-terminal cysteine residue at the protein N-terminus is attached to a fatty acid (R1) forming an amide linkage and the cysteinyl sulfhydryl group is attached to a glycerol moiety containing two-ester bound fatty acids (R2). The structure of R1 is deduced to be hexadecanoic acid (C16:0) and the structures of R2 vary depending on the rLP2086 isoforms.

The second polypeptide includes one modification introduced in the N-terminal region for the rLP2086 subfamily B protein, as compared to the corresponding wild-type sequence from N. meningitidis strain CDC-1573. A glycine in the second position is a consequence of introducing a cloning site.

The N-terminal differences from the original Neisserial sequences are shown below.

Comparison of Predicted N-Terminal Sequences of
Recombinant and Neisserial Subfamily B LP2086
Protein

| | |
|---|---|
| rLP2086 CDC- | CGSSGGGGSGGGGVTAD (SEQ ID NO: 24) |
| 1573 | |
| Neisserial | C-SSGGGGSGGGGVTAD (SEQ ID NO: 25) |
| LP2086 | |
| CDC-1573 | |

In one embodiment, the second polypeptide includes a C-G-S-S sequence (SEQ ID NO: 3) at the N-terminus. See the first four amino acid residues of SEQ ID NO: 2.

>B01
(SEQ ID NO: 2)
CGSSGGGGSGGGGVTADIGTGLADALTAPLDHKDKGLKSLTLEDSISQNG
TLTLSAQGAEKTYGNGDSLNTGKLKNDKVSRFDFIRQIEVDGQLITLESG
EFQVYKQSHSALTALQTEQEQDPEHSEKMVAKRRFRIGDIAGEHTSFDKL
PKDVMATYRGTAFGSDDAGGKLTYTIDFAAKQGHGKIEHLKSPELNVDLA
VAYIKPDEKHHAVISGSVLYNQDEKGSYSLGIFGEKAQEVAGSAEVETAN
GIHHIGLAAKQ

In one embodiment, the second polypeptide includes the amino acid sequence set forth in SEQ ID NO: 2. In one embodiment, the second polypeptide has a total of 261 amino acids. In one embodiment, the second polypeptide consists of the amino acid sequence set forth in SEQ ID NO: 2. In another embodiment, the second polypeptide does not further include a polypeptide having less than 100% sequence identity to SEQ ID NO: 2. In a preferred embodiment, the first polypeptide and the second polypeptide includes a C-G-S-S (SEQ ID NO: 3) sequence at the N-terminus of the respective polypeptide.

In a preferred embodiment, the second polypeptide is readily expressed in a recombinant host cell using standard techniques known in the art. In another preferred embodiment, the second polypeptide includes a bactericidal epitope on the N- and/or C-domain of SEQ ID NO: 2. In one embodiment, the second polypeptide includes at least the first 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues of the amino acid sequence set forth in SEQ ID NO: 2. Preferably, the second polypeptide includes at least the first 2, more preferably at least the first 4, and most preferably, at least the first 8 amino acid residues of SEQ ID NO: 2.

In another embodiment, the second polypeptide includes at least the last 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues of the amino acid sequence set forth in SEQ ID NO: 2.

In one embodiment, the composition includes about 30 μg/ml of a first polypeptide including the amino acid sequence set forth in SEQ ID NO: 2. In one preferred embodiment, the composition includes about 60 μg of a first polypeptide including the amino acid sequence set forth in SEQ ID NO: 2. In one preferred embodiment, the composition includes about 60 μg of a second polypeptide including the amino acid sequence set forth in SEQ ID NO: 2, wherein the composition preferably has a total volume of 0.5 ml. In another embodiment, the composition includes 120 μg/ml of a second polypeptide including the amino acid sequence set forth in SEQ ID NO: 2.

### Meningococcal Serogroups A, C, W, and Y (MenACWY) Capsular Saccharides

The term “saccharide” throughout this specification may indicate polysaccharide or oligosaccharide and includes both. Polysaccharides are isolated from bacteria or isolated from bacteria and sized to some degree by known methods and optionally by microfluidisation. Polysaccharides can be sized in order to reduce viscosity in polysaccharide samples and/or to improve filterability for conjugated products. Oligosaccharides have a low number of repeat units (typically 5-30 repeat units) and are typically hydrolysed polysaccharides.

Each N. meningitidis capsular saccharide may be conjugated to a carrier protein independently selected from the group consisting of TT, DT, CRM197, fragment C of TT and protein D. Although one or more N. meningitidis capsular saccharide may be conjugated to different carrier proteins from the others, in one embodiment they are all conjugated to the same carrier protein. For instance they may all be conjugated to the same carrier protein selected from the group consisting of TT, DT, CRM197, fragment C of TT and protein D. In this context CRM197 and DT may be considered to be the same carrier protein as they differ by only one amino acid. In a preferred embodiment all the N. meningitidis capsular saccharides present are conjugated to TT.

If the protein carrier is the same for 2 or more saccharides in the composition, the saccharide could be conjugated to the same molecule of the protein carrier (carrier molecules having 2 more different saccharides conjugated to it) [see for instance WO 04/083251; for example, a single carrier protein might be conjugated to MenA and MenC; MenA and MenW; MenA and MenY; MenC and MenW; MenC and MenY; Men W and MenY; MenA, MenC and MenW; MenA, MenC and MenY; MenA, MenW and MenY; MenC, MenW and MenY; MenA, MenC, MenW and MenY. Alternatively the saccharides may each be separately conjugated to different molecules of the protein carrier (each molecule of protein carrier only having one type of saccharide conjugated to it).

In one embodiment, at least 2 different saccharide conjugates are conjugated separately to the same type of carrier protein, wherein one or more saccharide(s) is/are conjugated to the carrier protein via a first type of chemical group on the protein carrier, and one or more saccharide(s) is/are conjugated to the carrier protein via a second (different) type of chemical group on the protein carrier.

In one embodiment the 2 conjugates involve the same saccharide linked to the same carrier, but by different conjugation chemistries. In an alternative embodiment 2 different saccharides are conjugated to different groups on the protein carrier.

By “conjugated separately to the same type of carrier protein” it is meant that the saccharides are conjugated to the same carrier individually (for example, MenA is conjugated to tetanus toxoid through an amine group on the tetanus toxoid and MenC is conjugated to tetanus toxoid through a carboxylic acid group on a different molecule of tetanus toxoid.)

The capsular saccharide(s) may be conjugated to the same carrier protein independently selected from the group consisting of TT, DT, CRM197, fragment C of TT and protein D. A more complete list of protein carriers that may be used in the conjugates of the disclosure is presented below. In this context CRM197 and DT may be considered to be the same carrier protein as they differ by only one amino acid. In an embodiment all the capsular saccharides present are conjugated to TT.

The saccharides may include any one of: N. meningitidis serogroup A capsular saccharide (MenA), N. meningitidis serogroup C capsular saccharide (MenC), N. meningitidis serogroup Y capsular saccharide (MenY), and N. meningitidis serogroup W capsular saccharide (MenW), or any combination thereof.

The first and second chemical groups present on the protein carrier are different from each other and are ideally natural chemical groups that may be readily used for conjugation purposes. They may be selected independently from the group consisting of: carboxyl groups, amino groups, sulphydryl groups, Hydroxyl groups, Imidazolyl groups, Guanidyl groups, and Indolyl groups. In one embodiment the first chemical group is carboxyl and the second is amino, or vice versa. These groups are explained in greater detail below.

In a specific embodiment the immunogenic composition comprises at least 2 different N. meningitidis capsular saccharides, wherein one or more is/are selected from a first group consisting of MenA and MenC which is/are conjugated to the carrier protein via the first type of chemical group on the protein carrier (for instance carboxyl), and one or more different saccharides is/are selected from a second group consisting of MenC, MenY and MenW which is/are conjugated to the carrier protein via the second type of chemical group on the protein carrier (for instance amino).

In a further embodiment the immunogenic composition of the disclosure comprises MenA conjugated via the first type of chemical group (for instance carboxyl), and MenC conjugated via the second type of chemical group (for instance amino).

In another embodiment the immunogenic composition comprises MenC conjugated via the first type of chemical group (for instance carboxyl), and MenY conjugated via the second type of chemical group (for instance amino).

In another embodiment the immunogenic composition comprises MenA conjugated via the first type of chemical group (for instance carboxyl), and MenC, MenY and MenW conjugated via the second type of chemical group (for instance amino).

In another embodiment the immunogenic composition comprises MenA and MenC conjugated via the first type of chemical group (for instance carboxyl), and MenY and MenW conjugated via the second type of chemical group (for instance amino).

The saccharides of the disclosure included in pharmaceutical (immunogenic) compositions of the disclosure are conjugated to a carrier protein such as tetanus toxoid (TT), tetanus toxoid fragment C, non-toxic mutants of tetanus toxin [note all such variants of TT are considered to be the same type of carrier protein for the purposes of this disclosure], diphtheria toxoid (DT), CRM197, other non-toxic mutants of diphtheria toxin [such as CRM176, CRM 197, CRM228, CRM 45 (Uchida et al J. Biol. Chem. 218; 3838-3844, 1973); CRM 9, CRM 45, CRM102, CRM 103 and CRM107 and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. No. 4,709,017 or U.S. Pat. No. 4,950,740; mutation of at least one or more residues Lys 516, Lys 526, Phe 530 and/or Lys 534 and other mutations disclosed in U.S. Pat. No. 5,917,017 or U.S. Pat. No. 6,455,673; or fragment disclosed in U.S. Pat. No. 5,843,711](note all such variants of DT are considered to be the same type of carrier protein for the purposes of this disclosure), pneumococcal pneumolysin (Kuo et al (1995) Infect Immun 63; 2706-13), OMPC (meningococcal outer membrane protein—usually extracted from N. meningitidis serogroup B—EP0372501), synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al (2001) Eur J Immunol 31; 3816-3824) such as N19 protein (Baraldoi et al (2004) Infect Immun 72; 4884-7) pneumococcal surface protein PspA (WO 02/091998), iron uptake proteins (WO 01/72337), toxin A or B of C. difficile (WO 00/61761) or Protein D (EP594610 and WO 00/56360).

In an embodiment, the immunogenic composition of the disclosure uses the same type of carrier protein (independently) in at least two, three, four or each of the saccharides contained therein.

In an embodiment, the immunogenic composition of the disclosure comprises a N. meningitidis saccharide conjugated to a carrier protein selected from the group consisting of TT, DT, CRM197, fragment C of TT and protein D.

The immunogenic composition of the disclosure optionally comprises at least one meningococcal saccharide (for example MenA; MenC; MenW; MenY; MenA and MenC; MenA and MenW; MenA and MenY; MenC and Men W; Men C and MenY; Men Wand MenY; MenA, MenC and MenW; MenA, MenC and MenY; MenA, MenW and MenY; MenC, MenW and MenY or MenA, MenC, MenW and MenY) conjugate having a ratio of Men saccharide to carrier protein of between 1:5 and 5:1, between 1:2 and 5:1, between 1:0.5 and 1:2.5 or between 1:1.25 and 1:2.5 (w/w). In one preferred embodiment, the composition includes MenA, MenC, MenW and MenY each conjugated to tetanus toxoid at ratios (toxoid to polysaccharide) of about 3, about 3, about 1.5 and about 1.3, respectively.

The ratio of saccharide to carrier protein (w/w) in a conjugate may be determined using the sterilized conjugate. The amount of protein is determined using a Lowry assay (for example Lowry et al (1951) J. Biol. Chem. 193, 265-275 or Peterson et al Analytical Biochemistry 100, 201-220 (1979)) and the amount of saccharide is determined using ICP-OES (inductively coupled plasma-optical emission spectroscopy) for MenA, DMAP assay for MenC and Resorcinol assay for MenW and MenY (Monsigny et al (1988) Anal. Biochem. 175, 525-530).

In an embodiment, the immunogenic composition of the disclosure comprises N. meningitidis saccharide conjugate(s) wherein the N. meningitidis saccharide(s) is conjugated to the carrier protein via a linker, for instance a bifunctional linker. The linker is optionally heterobifunctional or homobifunctional, having for example a reactive amino group and a reactive carboxylic acid group, 2 reactive amino groups or two reactive carboxylic acid groups. The linker has for example between 4 and 20, 4 and 12, 5 and 10 carbon atoms. A possible linker is ADH. Other linkers include B-propionamido (WO 00/10599), nitrophenyl-ethylamine (Gever et al (1979) Med. Microbiol. Immunol. 165; 171-288), haloalkyl halides (U.S. Pat. No. 4,057,685), glycosidic linkages (U.S. Pat. Nos. 4,673,574, 4,808,700), hexane diamine and 6-aminocaproic acid (U.S. Pat. No. 4,459,286).

The saccharide conjugates present in the immunogenic compositions of the disclosure may be prepared by any known coupling technique. The conjugation method may rely on activation of the saccharide with 1-cyano-4-dimethylamino pyridinium tetrafluoroborate (CDAP) to form a cyanate ester. The activated saccharide may thus be coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using GMBS) or a holoacetylated carrier protein (for example using iodoacetimide or N-succinimidyl bromoacetatebromoacetate). Optionally, the cyanate ester (optionally made by CDAP chemistry) is coupled with hexane diamine or ADH and the amino-derivatised saccharide is conjugated to the carrier protein using carbodiimide (e.g. EDAC or EDC) chemistry via a carboxyl group on the protein carrier. Such conjugates are described in PCT published application WO 93/15760 Uniformed Services University and WO 95/08348 and WO 96/29094.

Other suitable techniques use carbiinides, hydrazides, active esters, norborane, p-nitrobenzoic acid, N-hydroxysuccinimide, S-NHS, EDC, TSTU. Many are described in WO 98/42721. Conjugation may involve a carbonyl linker which may be formed by reaction of a free hydroxyl group of the saccharide with CDI (Bethell et al J. Biol. Chem. 1979, 254; 2572-4, Hearn et al J. Chromatogr. 1981. 218; 509-18) followed by reaction of with a protein to form a carbamate linkage. This may involve reduction of the anomeric terminus to a primary hydroxyl group, optional protection/deprotection of the primary hydroxyl group' reaction of the primary hydroxyl group with CDI to form a CDI carbamate intermediate and coupling the CDI carbamate intermediate with an amino group on a protein.

The conjugates can also be prepared by direct reductive amination methods as described in U.S. Pat. No. 4,365,170 (Jennings) and U.S. Pat. No. 4,673,574 (Anderson). Other methods are described in EP-0-161-188, EP-208375 and EP-0-477508.

A further method involves the coupling of a cyanogen bromide (or CDAP) activated saccharide derivatised with adipic acid hydrazide (ADH) to the protein carrier by Carbodiimide condensation (Chu C. et al Infect. Immunity, 1983 245 256), for example using EDAC.

In an embodiment, a hydroxyl group (optionally an activated hydroxyl group for example a hydroxyl group activated by a cyanate ester) on a saccharide is linked to an amino or carboxylic group on a protein either directly or indirectly (through a linker). Where a linker is present, a hydroxyl group on a saccharide is optionally linked to an amino group on a linker, for example by using CDAP conjugation. A further amino group in the linker for example ADH) may be conjugated to a carboxylic acid group on a protein, for example by using carbodiimide chemistry, for example by using EDAC. In an embodiment, N. meningitidis capsular saccharide(s) (or saccharide in general) is conjugated to the linker first before the linker is conjugated to the carrier protein. Alternatively the linker may be conjugated to the carrier before conjugation to the saccharide.

In general the following types of chemical groups on a protein carrier can be used for coupling/conjugation:
A) Carboxyl (for instance via aspartic acid or glutamic acid). In one embodiment this group is linked to amino groups on saccharides directly or to an amino group on a linker with carbodiimide chemistry e.g. with EDAC.
B) Amino group (for instance via lysine). In one embodiment this group is linked to carboxyl groups on saccharides directly or to a carboxyl group on a linker with carbodiimide chemistry e.g. with EDAC. In another embodiment this group is linked to hydroxyl groups activated with CDAP or CNBr on saccharides directly or to such groups on a linker; to saccharides or linkers having an aldehyde group; to saccharides or linkers having a succinimide ester group.
C) Sulphydryl (for instance via cysteine). In one embodiment this group is linked to a bromo or chloro acetylated saccharide or linker with maleimide chemistry. In one embodiment this group is activated/modified with bis diazobenzidine.
D) Hydroxyl group (for instance via tyrosine). In one embodiment this group is activated/modified with bis diazobenzidine.
E) Imidazolyl group (for instance via histidine). In one embodiment this group is activated/modified with bis diazobenzidine.
F) Guanidyl group (for instance via arginine).
G) Indolyl group (for instance via tryptophan).

On a saccharide, in general the following groups can be used for a coupling: OH, COOH or NH2. Aldehyde groups can be generated after different treatments known in the art such as: periodate, acid hydrolysis, hydrogen peroxide, etc.

### Direct Coupling Approaches:

Saccharide-OH+CNBr or CDAP----->cyanate ester+NH2-Prot---->conjugate
Saccharide-aldehyde+NH2-Prot---->Schiff base+NaCNBH3---->conjugate
Saccharide-COOH+NH2-Prot+EDAC---->conjugate
Saccharide-NH2+COOH-Prot+EDAC---->conjugate

### Indirect Coupling Via Spacer (Linker) Approaches:

Saccharide-OH+CNBr or CDAP--->cyanate ester+NH2----NH2---->saccharide----NH2+COOH-Prot+EDAC----->conjugate
Saccharide-OH+CNBr or CDAP---->cyanate ester+NH2-----SH----->saccharide----SH+SH-Prot (native Protein with an exposed cysteine or obtained after modification of amino groups of the protein by SPDP for instance)----->saccharide-S—S-Prot
Saccharide-OH+CNBr or CDAP--->cyanate ester+NH2----SH------->saccharide----SH+maleimide-Prot (modification of amino groups)---->conjugate
Saccharide-COOH+EDAC+NH2-----NH2--->saccharide------NH2+EDAC+COOH-Prot---->conjugate
Saccharide-COOH+EDAC+NH2----SH----->saccharide----SH+SH-Prot (native Protein with an exposed cysteine or obtained after modification of amino groups of the protein by SPDP for instance)----->saccharide-S—S-Prot
Saccharide-COOH+EDAC+NH2----SH----->saccharide----SH+maleimide-Prot (modification of amino groups)---->conjugate
Saccharide-Aldehyde+NH2-----NH2---->saccharide---NH2+EDAC+COOH-Prot---->conjugate
   Note: instead of EDAC above, any suitable carbodiimide may be used.

In summary, the types of protein carrier chemical group that may be generally used for coupling with a saccharide are amino groups (for instance on lysine residues), COOH groups (for instance on aspartic and glutamic acid residues) and SH groups (if accessible) (for instance on cysteine residues).

In an embodiment, at least one of the N. meningitidis capsular saccharides (or saccharide in general) is directly conjugated to a carrier protein; optionally Men W and/or MenY and/or MenC saccharide(s) is directly conjugated to a carrier protein. For example MenW; MenY; MenC; MenW and MenY; MenW and MenC; MenY and MenC; or MenW, MenY and MenC are directly linked to the carrier protein. Optionally, at least one of the N. meningitidis capsular saccharides is directly conjugated by CDAP. For example MenW; MenY; MenC; MenW and MenY; MenW and MenC; MenY and MenC; or MenW, MenY and MenC are directly linked to the carrier protein by CDAP (see WO 95/08348 and WO 96/29094). In an embodiment, all N. meningitidis capsular saccharides are conjugated to tetanus toxoid.

In an embodiment, the ratio of Men W and/or Y saccharide to carrier protein is between 1:0.5 and 1:2 (w/w) and/or the ratio of MenC saccharide to carrier protein is between 1:0.5 and 1:4 or 1:0.5 and 1:1.5 (w/w), especially where these saccharides are directly linked to the protein, optionally using CDAP.

In an embodiment, at least one of the N. meningitidis capsular saccharide(s) (or saccharide in general) is conjugated to the carrier protein via a linker, for instance a bifunctional linker. The linker is optionally heterobifunctional or homobifunctional, having for example a reactive amine group and a reactive carboxylic acid group, 2 reactive amine groups or 2 reactive carboxylic acid groups. The linker has for example between 4 and 20, 4 and 12, 5 and 10 carbon atoms. A possible linker is ADH.

In an embodiment, MenA; MenC; or MenA and MenC is conjugated to a carrier protein (for example tetanus toxoid) via a linker.

In an embodiment, at least one N. meningitidis saccharide is conjugated to a carrier protein via a linker using CDAP and EDAC. For example, MenA; MenC; or MenA and MenC are conjugated to a protein via a linker (for example those with two hydrazino groups at its ends such as ADH) using CDAP and EDAC as described above. For example, CDAP is used to conjugate the saccharide to a linker and EDAC is used to conjugate the linker to a protein. Optionally the conjugation via a linker results in a ratio of saccharide to carrier protein of between 1:0.5 and 1:6; 1:1 and 1:5 or 1:2 and 1:4, for MenA; MenC; or MenA and MenC.

In an embodiment, the MenA capsular saccharide, where present is at least partially 0-acetylated such that at least 50%, 60%, 70%, 80%, 90%, 95% or 98% of the repeat units are O-acetylated at at least one position. O-acetylation is for example present at least at the 0-3 position of at least 50%, 60%, 70%, 80%, 90%, 95% or 98% of the repeat units.

In an embodiment, the MenC capsular saccharide, where present is at least partially O-acetylated such that at least 30%, 40%, 50%, 60%, 70%, 80%, 90%, 95% or 98% of (α2→9)-linked NeuNAc repeat units are O-acetylated at at least one or two positions. O-acetylation is for example present at the 0-7 and/or 0-8 position of at least 30%. 40%, 50%, 60%, 70%, 80%, 90%, 95% or 98% of the repeat units.

In an embodiment, the MenW capsular saccharide, where present is at least partially O-acetylated such that at least 30%, 40%, 50%, 60%, 70%, 80%, 90%, 95% or 98% of the repeat units are O-acetylated at at least one or two positions. O-acetylation is for example present at the 0-7 and/or 0-9 position of at least 30%, 40%, 50%, 60%, 70%, 80%, 90%, 95% or 98% of the repeat units.

In an embodiment, the MenY capsular saccharide, where present is at least partially O-acetylated such that at least 20%, 30%, 40%, 50%, 60%, 70%, 80%, 90%, 95% or 98% of the repeat units are O-acetylated at at least one or two positions. O-acetylation is present at the 7 and/or 9 position of at least 20%, 30%, 40%, 50%, 60%, 70%, 80%, 90%, 95% or 98% of the repeat units.

The percentage of O-acetylation refers to the percentage of the repeat units containing O-acetylation. This may be measured in the saccharide prior to conjugate and/or after conjugation.

In one embodiment of the disclosure the immunogenic composition, saccharide present, or each N. meningitidis capsular saccharide present, is conjugated to TT. In a further embodiment each N. meningitidis capsular saccharide is separately conjugated to a separate carrier protein. In a further embodiment each N. meningitidis capsular saccharide conjugate has a saccharide:carrier ratio of 1:5-5:1 or 1:1-1:4 (w/w). In a further embodiment at least one, two or three N. meningitidis capsular saccharide conjugate(s) is directly conjugated to a carrier protein. In a further embodiment Men W and/or MenY, MenW and/or MenC, MenY and/or MenC, or MenW and MenC and MenY are directly conjugated to a carrier protein. In a further embodiment at least one, two or three N. meningitidis saccharide conjugate(s) is directly conjugated by CDAP chemistry. In a further embodiment the ratio of Men W and/or Y saccharide to carrier protein is between 1:0.5 and 1:2 (w/w). In a further embodiment the ratio of MenC saccharide to carrier protein is between 1:0.5 and 1:2 (w/w). In a further embodiment at least one, two or three N. meningitidis capsular saccharide(s) are conjugated to the carrier protein via a linker (which may be bifunctional such as having two reactive amino groups (such as ADH) or two reactive carboxyl groups, or a reactive amino group at one end and a reactive carboxyl group at the other). The linker can have between 4 and 12 carbon atoms. In a further embodiment the or each N. meningitidis capsular saccharide(s) conjugated via a linker are conjugated to the linker with CDAP chemistry. In a further embodiment the carrier protein is conjugated to the linker using carbodiimide chemistry, for example using EDAC. In a further embodiment the or each N. meningitidis capsular saccharide is conjugated to the linker before the carrier protein is conjugated to the linker. In a further embodiment MenA is conjugated to a carrier protein via a linker (the ratio of MenA saccharide to carrier protein may be between 1:2 and 1:5 (w/w)). In a further embodiment MenC is conjugated to a carrier protein via a linker (the ratio of MenC saccharide to carrier protein may be between 1:2 and 1:5 (w/w)).

By using native or slightly sized polysaccharide conjugates, one or more of the following advantages may be realised: 1) a conjugate having high immunogenicity which is filterable through a 0.2 micron filter; 2) immune memory may be enhanced (as in example three); 3) the alteration of the ratio of polysaccharide to protein in the conjugate such that the ratio of polysaccharide to protein (w/w) in the conjugate may be increased (this can result in a reduction of the carrier suppression effect); 4) immunogenic conjugates prone to hydrolysis (such as MenA conjugates) may be stabilised by the use of larger polysaccharides for conjugation. The use of larger polysaccharides can result in more cross-linking with the conjugate carrier and may lessen the liberation of free saccharide from the conjugate. The conjugate vaccines described in the prior art tend to depolymerise the polysaccharides prior to conjugation in order to improve conjugation. Meningococcal (or saccharide) conjugate vaccines retaining a larger size of saccharide can provide a good immune response against meningococcal disease.

The immunogenic composition of the disclosure may thus comprise one or more saccharide conjugates wherein the average size of each saccharide before conjugation is above 50 kDa, 75 kDa, 100 kDa, 110 kDa, 120 kDa or 130 kDa. In one embodiment the conjugate post conjugation should be readily filterable through a 0.2 micron filter such that a yield of more than 50, 60, 70, 80, 90 or 95% is obtained post filtration compared with the pre filtration sample.

In particular, the immunogenic composition of the disclosure comprises N. meningitidis capsular saccharides from at least one, two, three or four of serogroups A, C, W and Y conjugated to a carrier protein, wherein the average size (weight-average molecular weight; Mw) of at least one, two, three or four or each N. meningitidis saccharide is above 50 kDa, 60 kDa, 75 kDa, 100 kDa, 110 kDa, 120 kDa or 130 kDa.

In a preferred embodiment, the average Mw of the MenA_{AH}-TT conjugate is at least 250 kDa, 260 kDa, 270 kDa, 280 kDa, or 290 kDa, most preferably about 300 kDa, and at most 350 kDa or 330 kDa. In a preferred embodiment, the average Mw of the MenC_{AH}-TT conjugate is at least 150 kDa, 160 kDa, 170 kDa, 180 kDa, or 190 kDa, most preferably about 200 kDa, and at most 250 kDa or 230 kDa. In a preferred embodiment, the average Mw of the MenW-TT conjugate is at least 240, 250 kDa, 260 kDa, or 270 kDa, most preferably about 280 kDa, and at most 330 kDa or 310 kDa. In a preferred embodiment, the average Mw of the MenY-TT conjugate is at least 220 kDa, 230 kDa, 240 kDa, or 250 kDa, most preferably about 270 kDa, and at most 320 kDa or 300 kDa.

The immunogenic composition may comprise N. meningitidis capsular saccharides from at least one, two, three or four of serogroups A, C, W and Y conjugated to a carrier protein, wherein at least one, two, three or four or each N. meningitidis saccharide is either a native saccharide or is sized by a factor up to ×2, ×3, ×4, ×5, ×6, ×7, ×8, ×9 or ×10 relative to the weight average molecular weight of the native polysaccharide.

For the purposes of the disclosure, “native polysaccharide” refers to a saccharide that has not been subjected to a process, the purpose of which is to reduce the size of the saccharide. A polysaccharide can become slightly reduced in size during normal purification procedures. Such a saccharide is still native. Only if the polysaccharide has been subjected to sizing techniques would the polysaccharide not be considered native.

For the purposes of the disclosure, “sized by a factor up to ×2” means that the saccharide is subject to a process intended to reduce the size of the saccharide but to retain a size more than half the size of the native polysaccharide. ×3, ×4 etc. are to be interpreted in the same way i.e. the saccharide is subject to a process intended to reduce the size of the polysaccharide but to retain a size more than a third, a quarter etc. the size of the native polysaccharide.

In an aspect of the disclosure, the immunogenic composition comprises N. meningitidis capsular saccharides from at least one, two, three or four of serogroups A, C, W and Y conjugated to a carrier protein, wherein at least one, two, three or four or each N. meningitidis saccharide is native polysaccharide.

In an aspect of the disclosure, the immunogenic composition comprises N. meningitidis capsular saccharides from at least one, two, three or four of serogroups A, C, W and Y conjugated to a carrier protein, wherein at least one, two, three or four or each N. meningitidis saccharide is sized by a factor up to ×1.5, ×2, ×3, ×4, ×5, ×6, ×7, ×8, ×9 or ×10. The immunogenic compositions of the disclosure optionally comprise conjugates of: N. meningitidis serogroup C capsular saccharide (MenC), serogroup A capsular saccharide (MenA), serogroup W135 capsular saccharide (MenW), serogroup Y capsular saccharide (MenY), serogroup C and Y capsular saccharides (MenCY), serogroup C and A capsular saccharides (MenAC), serogroup C and W capsular saccharides (MenCW), serogroup A and Y capsular saccharide (MenAY), serogroup A and W capsular saccharides (MenAW), serogroup W and Y capsular saccharides (Men WY), serogroup A, C and W capsular saccharide (MenACW), serogroup A, C and Y capsular saccharides (MenACY); serogroup A, W135 and Y capsular saccharides (MenAWY), serogroup C, W135 and Y capsular saccharides (MenCWY); or serogroup A, C, W135 and Y capsular saccharides (MenACWY). This is the definition of “one, two, three or four”, or “at least one of” of serogroups A, C, W and Y, or of each N. meningitidis saccharide where mentioned herein.

In an embodiment, the average size of at least one, two, three, four or each N. meningitidis saccharide is between 50 KDa and 1500 kDa, 50 kDa and 500 kDa, 50 kDa and 300 KDa, 101 kDa and 1500 kDa, 101 kDa and 500 kDa, 101 kDa and 300 kDa as determined by MALLS.

In an embodiment, the MenA saccharide, where present, has a molecular weight of 50-500 kDa, 50-100 kDa, 100-500 kDa, 55-90 KDa, 60-70 kDa or 70-80 kDa or 60-80 kDa. In an embodiment, the MenC saccharide, where present, has a molecular weight of 100-200 kDa, 50-100 kDa, 100-150 kDa, 101-130 kDa, 150-210 kDa or 180-210 kDa.

In an embodiment the MenY saccharide, where present, has a molecular weight of 60-190 kDa, 70-180 kDa, 80-170 kDa, 90-160 kDa, 100-150 kDa or 110-140 kDa, 50-100 kDa, 100-140 kDa, 140-170 kDa or 150-160 kDa.

In an embodiment the MenW saccharide, where present, has a molecular weight of 60-190 kDa, 70-180 kDa, 80-170 kDa, 90-160 kDa, 100-150 kDa, 110-140 kDa, 50-100 kDa or 120-140 kDa.

The molecular weight or average molecular weight of a saccharide herein refers to the weight-average molecular weight (Mw) of the saccharide measured prior to conjugation and is measured by MALLS.

The MALLS technique is well known in the art and is typically carried out as described in example 2. For MALLS analysis of meningococcal saccharides, two columns (TSKG6000 and 5000PWxl) may be used in combination and the saccharides are eluted in water. Saccharides are detected using a light scattering detector (for instance Wyatt Dawn DSP equipped with a 10 mW argon laser at 488 nm) and an inferometric refractometer (for instance Wyatt Otilab DSP equipped with a P100 cell and a red filter at 498 nm).

In an embodiment the N. meningitidis saccharides are native polysaccharides or native polysaccharides which have reduced in size during a normal extraction process.

In an embodiment, the N. meningitidis saccharides are sized by mechanical cleavage, for instance by microfluidisation or sonication. Microfluidisation and sonication have the advantage of decreasing the size of the larger native polysaccharides sufficiently to provide a filterable conjugate (for example through a 0.2 micron filter). Sizing is by a factor of no more than ×20, ×10, ×8, ×6, ×5, ×4, ×3, ×2 or ×1.5.

In an embodiment, the immunogenic composition comprises N. meningitidis conjugates that are made from a mixture of native polysaccharides and saccharides that are sized by a factor of no more than ×20. For example, saccharides from MenC and/or MenA are native. For example, saccharides from MenY and/or MenW are sized by a factor of no more than ×20, ×10, ×8, ×6, ×5, ×4, ×3 or ×2. For example, an immunogenic composition contains a conjugate made from MenY and/or MenW and/or MenC and/or MenA which is sized by a factor of no more then ×10 and/or is microfluidised. For example, an immunogenic composition contains a conjugate made from native MenA and/or MenC and/or MenW and/or MenY. For example, an immunogenic composition comprises a conjugate made from native MenC. For example, an immunogenic composition comprises a conjugate made from native MenC and MenA which is sized by a factor of no more then ×10 and/or is microfluidised. For example, an immunogenic composition comprises a conjugate made from native MenC and MenY which is sized by a factor of no more then ×10 and/or is microfluidised.

In an embodiment, the polydispersity of the saccharide is 1-1.5, 1-1.3, 1-1.2, 1-1.1 or 1-1.05 and after conjugation to a carrier protein, the polydispersity of the conjugate is 1.0-2.5, 1.0-2.0. 1.0-1.5, 1.0-1.2, 1.5-2.5, 1.7-2.2 or 1.5-2.0. All polydispersity measurements are by MALLS.

Saccharides are optionally sized up to 1.5, 2, 4, 6, 8, 10, 12, 14, 16, 18 or 20 times from the size of the polysaccharide isolated from bacteria.

In one embodiment each N. meningitidis saccharide is either a native polysaccharide or is sized by a factor of no more than ×10. In a further embodiment each N. meningitidis capsular saccharide is a native polysaccharide. In a further embodiment at least one, two, three or four N. meningitidis capsular saccharide(s) is sized by microfluidization. In a further embodiment each N. meningitidis capsular saccharide is sized by a factor of no more than ×10. In a further embodiment the N. meningitidis conjugates are made from a mixture of native polysaccharides and saccharides that are sized by a factor of no more than ×10. In a further embodiment the capsular saccharide from serogroup Y is sized by a factor of no more than ×10. In a further embodiment capsular saccharides from serogroups A and C are native polysaccharides and saccharides from serogroups W135 and Y are sized by a factor of no more than ×10. In a further embodiment the average size of each N. meningitidis capsular saccharide is between 50 kDa and 300 KDa or 50 kDa and 200 kDa. In a further embodiment the immunogenic composition comprises a MenA capsular saccharide having an average size of above 50 kDa, 75 kDa, 100 kDa or an average size of between 50-100 kDa or 55-90 KDa or 60-80 kDa. In a further embodiment the immunogenic composition comprises a MenC capsular saccharide having an average size of above 50 kDa, 75 kDa, 100 kDa or between 100-200 kDa, 100-150 kDa, 80-120 kDa, 90-110 kDa, 150-200 kDa, 120-240 kDa, 140-220 kDa, 160-200 kDa or 190-200 kDa. In a further embodiment the immunogenic composition comprises a MenY capsular saccharide, having an average size of above 50 kDa, 75 kDa, 100 kDa or between 60-190 kDa or 70-180 kDa or 80-170 kDa or 90-160 kDa or 100-150 kDa, 110-145 kDa or 120-140 kDa. In a further embodiment the immunogenic composition comprises a MenW capsular saccharide having an average size of above 50 kDa, 75 kDa, 100 kDa or between 60-190 kDa or 70-180 kDa or 80-170 kDa or 90-160 kDa or 100-150 kDa, 140-180 kDa, 150-170 kDa or 110-140 kDa.

In an embodiment of the disclosure, the saccharide dose of each of the at least two, three, four or each of the N. meningitidis saccharide conjugates is optionally the same, or approximately the same.

In an embodiment, the immunogenic composition of the disclosure is adjusted to or buffered at, or adjusted to between pH 7.0 and 8.0, pH 7.2 and 7.6 or around or exactly pH 7.4.

The immunogenic composition or vaccines of the disclosure are optionally lyophilised in the presence of a stabilising agent for example a polyol such as sucrose or trehalose.

For the N. meningitidis saccharide combinations discussed above, it may be advantageous not to use any aluminium salt adjuvant or any adjuvant at all.

The active agent can be present in varying concentrations in the pharmaceutical composition or vaccine of the disclosure. Typically, the minimum concentration of the substance is an amount necessary to achieve its intended use, while the maximum concentration is the maximum amount that will remain in solution or homogeneously suspended within the initial mixture. For instance, the minimum amount of a therapeutic agent is optionally one which will provide a single therapeutically effective dosage. For bioactive substances, the minimum concentration is an amount necessary for bioactivity upon reconstitution and the maximum concentration is at the point at which a homogeneous suspension cannot be maintained.

In another embodiment, the composition includes a conjugate of a Neisseria meningitidis serogroup X capsular polysaccharide and a carrier molecule. The structure of the group X capsular polysaccharide consists of N-acetylglucosamine-4-phosphate residues held together by al-4 phosphodiester bonds without 0-acetyl groups. The carrier molecule may be a diphtheria or tetanus toxoid, CRM 197 or protein D. In a preferred embodiment, as exemplified in the Examples, the composition does not include a conjugate of a N. meningitidis serogroup X capsular polysaccharide.

Further descriptions of exemplary compositions are described below.

### Composition and Vaccine

In some embodiments, the composition includes a lyophilized MenACWY-TT composition that is reconstituted with a liquid MnB bivalent rLP2086 composition. The lyophilized MenACWY-TT composition and the liquid MnB bivalent rLP2086 composition are preferably compatible and stable following reconstitution for at least 24 hours at room temperature.

In preferred embodiments, the composition elicits a bactericidal antibody against N. meningitidis serogroup B and N. meningitidis serogroups other than B. For example, in some embodiments, the MnB bivalent rLP2086 composition elicits a bactericidal antibody against at least N. meningitidis serogroups A, C, W, Y, and X.

Furthermore, the inventors discovered that the composition elicited a geometric mean titer against N. meningitidis serogroups A, C, W, and Y that is consistent with the geometric mean titer observed for a licensed vaccine against N. meningitidis serogroups A, C, W, and Y.

In some embodiments, the composition elicits a geometric mean titer against N. meningitidis serogroups A, C, W, and Y that is higher than the geometric mean titer observed for a licensed vaccine against N. meningitidis serogroups A, C, W, and Y.

Moreover, the inventors discovered that the composition elicited a geometric mean titer against N. meningitidis serogroup B that is consistent with a geometric mean titer observed for a licensed vaccine against N. meningitidis serogroup B.

In some embodiments, the composition elicits a geometric mean titer against N. meningitidis serogroup B that is higher than the geometric mean titer observed for a licensed vaccine against N. meningitidis serogroup B.

In some embodiments, the composition including fHBP elicits an effective immune response in humans aged at least 12 months. The composition also elicits an immune response against a N. meningitidis serogroup X strain. In some embodiments, the composition includes at least one factor H binding polypeptide (fHBP) and at least one N. meningitidis capsular saccharide conjugate. In a preferred embodiment, the composition is stable and elicited an immune response against strains that express fHBP variants that are homologous to the fHBP variant in the multi-component composition and an immune response against strains that express fHBP variants that are heterologous to the fHBP variant in the multi-component composition.

In some embodiments, the liquid MnB bivalent rLP2086 composition can readily reconstitute a lyophilized MenACWY-TT composition and the combined composition is compatible and stable.

In one aspect, the disclosure relates to a composition against Neisseria meningitidis. The composition includes (a) a first lipidated polypeptide including the amino acid sequence set forth in SEQ ID NO: 1; (b) a second lipidated polypeptide including the amino acid sequence set forth in SEQ ID NO: 2; (c) a Neisseria meningitidis serogroup A (MenA) capsular saccharide conjugated to an adipic acid dihydrazide (ADH) linker by 1-cyano-4-dimethylamino pyridinium tetrafluoroborate chemistry, wherein the linker is conjugated to tetanus toxoid carrier protein (TT) by carbodiimide chemistry (MenA_{AH}-TT conjugate); (d) a Neisseria meningitidis serogroup C (MenC) capsular saccharide conjugated to an ADH linker by 1-cyano-4-dimethylamino pyridinium tetrafluoroborate chemistry, wherein the linker is conjugated to tetanus toxoid carrier protein (TT) by carbodiimide chemistry (MenC_{AH}-TT conjugate); (e) a Neisseria meningitidis serogroup W₁₃₅ (MenW) capsular saccharide directly conjugated to tetanus toxoid carrier protein (TT) by 1-cyano-4-dimethylamino pyridinium tetrafluoroborate chemistry, in the absence of a linker (MenW-TT conjugate); (f) a Neisseria meningitidis serogroup Y (MenY) capsular saccharide directly conjugated to tetanus toxoid carrier protein (TT) by 1-cyano-4-dimethylamino pyridinium tetrafluoroborate chemistry, in the absence of a linker (MenY-TT conjugate).

In another aspect, the disclosure relates to a composition that includes a combination of a MnB bivalent rLP2086 composition and a MenACWY-TT composition. The MnB bivalent rLP2086 composition refers to a composition that includes a single N. meningitidis polypeptide component that induces an effective broadly protective immune response against multiple strains of N. meningitidis serogroup B. Specifically, in one embodiment, the MnB bivalent rLP2086 composition includes a MnB rLP2086 subfamily A protein (SEQ ID NO: 1) and MnB rLP2086 subfamily B protein (SEQ ID NO: 2). In one embodiment, the composition does not include a fusion protein. In one embodiment, the composition does not include a chimeric protein. In one embodiment, the composition does not include a hybrid protein. In one embodiment, the composition does not further include a peptide fragment. In another embodiment, the composition does not further include a Neisserial polypeptide that is not fHBP. For example, in one embodiment, the composition does not include a PorA protein. In another embodiment, the composition does not include a NadA protein. In another embodiment, the composition does not further include a Neisserial heparin binding antigen (NHBA). In another embodiment, the composition does not further include a Neisserial outer membrane vesicle (OMV). In a preferred embodiment, the composition does not further include antigens, other than the first polypeptide and the second polypeptide. In a preferred embodiment, the MnB bivalent rLP2086 composition further includes polysorbate-80. In one embodiment, the MnB bivalent rLP2086 composition further includes histidine buffer. In one embodiment, the MnB bivalent rLP2086 composition further includes sodium chloride. In one embodiment, the MnB bivalent rLP2086 composition further includes aluminum phosphate. In one embodiment, the MnB bivalent rLP2086 composition further includes polysorbate-80, histidine buffer, sodium chloride, and aluminum phosphate. Preferably, the MnB bivalent rLP2086 composition is a liquid formulation, wherein the polypeptides are formulated as 120 mcg/mL/subfamily in 10 mM histidine buffer, pH 6.0, 150 mM sodium chloride (NaCl) with 0.5 mg/mL aluminum phosphate (AlPO₄), and further includes 0.018 mg polysorbate-80 in a 0.5 mL dose.

The MenACWY-TT composition refers to a composition that includes purified capsular polysaccharides of Neisseria meningitidis Serogroup A, C, W-135 and Y, each independently conjugated to TT at ratios (TT to polysaccharide) of ˜3˜, 3, ˜1.5 and ˜1.3, respectively. Specifically, the composition includes (c) a Neisseria meningitidis serogroup A (MenA) capsular saccharide conjugated to an adipic acid dihydrazide (ADH) linker by 1-cyano-4-dimethylamino pyridinium tetrafluoroborate chemistry, wherein the linker is conjugated to tetanus toxoid carrier protein (TT) by carbodiimide chemistry (MenA_{AH}-TT conjugate); (d) a Neisseria meningitidis serogroup C (MenC) capsular saccharide conjugated to an ADH linker by 1-cyano-4-dimethylamino pyridinium tetrafluoroborate chemistry, wherein the linker is conjugated to tetanus toxoid carrier protein (TT) by carbodiimide chemistry (MenC_{AH}-TT conjugate); (e) a Neisseria meningitidis serogroup W₁₃₅ (MenW) capsular saccharide directly conjugated to tetanus toxoid carrier protein (TT) by 1-cyano-4-dimethylamino pyridinium tetrafluoroborate chemistry, in the absence of a linker (MenW-TT conjugate); (f) a Neisseria meningitidis serogroup Y (MenY) capsular saccharide directly conjugated to tetanus toxoid carrier protein (TT) by 1-cyano-4-dimethylamino pyridinium tetrafluoroborate chemistry, in the absence of a linker (MenY-TT conjugate). Preferably, the MenACWY-TT composition is presented as a lyophilized powder.

MenA_{AH}-TT, MenC_{AH}-TT, MenW-TT, and MenY-TT conjugates are prepared through the following steps: manufacture of the polysaccharide drug substance intermediate, manufacture of the TT drug substance intermediate, microfluidization of the polysaccharide, derivatization of the polysaccharide (for the MenAAH-TT and MenCAH-TT processes only), additional purification of the TT, and conjugation of the individual polysaccharides to TT.

Regarding the MenA_{AH}-TT conjugate, the MenA polysaccharide is first microfluidized to reduce molecular size and viscosity, then activated via cyanylation with 1-cyano-4-dimethylamino-pyridinium tetrafluoroborate (CDAP). Activated MenA is derivatized with adipic acid dihydrazide (ADH) to form the MenA_{AH}. MenA_{AH} and Tetanus Toxoid (TT) are coupled through carbodiimide-mediated condensation (1-ethyl-3-(3-dimethylaminopropyl)carbodiimide (EDAC) coupling technology) to form MenA_{AH}-Tetanus Toxoid Conjugate (MenA_{AH}-TT).

Regarding the MenC_{AH}-TT conjugate, the MenC polysaccharide is first microfluidized to reduce molecular size and viscosity, then activated via cyanylation with CDAP. Activated MenC is derivatized with adipic acid dihydrazide (ADH) to form the MenC_{AH}. MenC_{AH} and TT are coupled through carbodiimide-mediated condensation EDAC coupling technology) to form MenC_{AH}-Tetanus Toxoid (MenC_{AH}-TT).

Regarding the MenW-TT conjugate, MenW polysaccharide is first microfluidized to reduce molecular size and viscosity, then activated via cyanylation with CDAP. Activated MenW is directly coupled to TT to form MenW-Tetanus Toxoid (MenW-TT).

Regarding the MenY-TT conjugate, MenY polysaccharide is first microfluidized to reduce molecular size and viscosity, then activated via cyanylation with CDAP. Activated MenY is directly coupled to TT to form MenY-Tetanus Toxoid (MenY-TT).

In another aspect, the polypeptide antigens are derived from at most two N. meningitidis serogroup B strains induces an effective broadly protective immune response against multiple strains of N. meningitidis serogroup B. Accordingly, in one embodiment, the composition does not further include a polypeptide that is not derived from N. meningitidis serogroup B fHBP subfamily A M98250771 strain and/or N. meningitidis serogroup B fHBP subfamily B CDC1573 strain.

In one embodiment, the composition does not further include a polypeptide having less than 100% sequence identity to SEQ ID NO: 1. In another embodiment, the composition does not further include a polypeptide having less than 100% sequence identity to SEQ ID NO: 2. For example, the composition does not further include a polypeptide having less than 100% sequence identity to the full length of SEQ ID NO: 1 and/or SEQ ID NO: 2.

In one embodiment, the composition further includes polysorbate-80, aluminum, histidine, and sodium chloride. In one embodiment, the composition includes about 60 μg of a first lipidated polypeptide including the amino acid sequence set forth in SEQ ID NO: 1, about 60 μg of a second lipidated polypeptide including the amino acid sequence set forth in SEQ ID NO: 2, 2.8 molar ratio of polysorbate-80 to each polypeptide, 0.5 mg aluminum/ml as aluminum phosphate, 10 mM histidine, and 150 mM sodium chloride, wherein the composition preferably has a total volume of about 0.5 ml.

In another aspect, the composition includes about 120 μg/ml of a first lipidated polypeptide including the amino acid sequence set forth in SEQ ID NO: 1, about 120 μg/ml of a second lipidated polypeptide including the amino acid sequence set forth in SEQ ID NO: 2, 2.8 molar ratio of polysorbate-80 to each polypeptide, 0.5 mg aluminum/ml as aluminum phosphate, 10 mM histidine, and 150 mM sodium chloride.

In a further aspect, the composition includes a) 60 μg of a first lipidated polypeptide including the amino acid sequence set forth in SEQ ID NO: 1; b) 60 μg of a second lipidated polypeptide including the amino acid sequence set forth in SEQ ID NO: 2; c) 18 μg polysorbate-80; d) 250 μg aluminum; e) 780 μg histidine, and; f) 4380 μg sodium chloride.

In an exemplary embodiment, the composition includes about 60 μg of a first lipidated polypeptide consisting of the amino acid sequence set forth in SEQ ID NO: 1, about 60 μg of a second lipidated polypeptide consisting of the amino acid sequence set forth in SEQ ID NO: 2, 2.8 molar ratio of polysorbate-80 to first lipidated polypeptide and to second lipidated polypeptide, 0.5 mg/ml aluminum phosphate, 10 mM histidine, and 150 mM sodium chloride, wherein the composition has a total volume of about 0.5 ml. In the exemplary embodiment, the composition is a sterile isotonic buffered liquid suspension. In the exemplary embodiment, the composition has a pH 6.0. In the exemplary embodiment, the first polypeptide and the second polypeptide are adsorbed to aluminum.

In one embodiment, the composition includes a MenA_{AH}-TT conjugate having a mean TT/polysaccharide ratio 3; a MenC_{AH}-TT conjugate having a mean TT/polysaccharide ratio 3; a MenW-TT conjugate having a mean TT/polysaccharide ratio 1.5; and a MenY-TT conjugate having a mean TT/polysaccharide ratio 1.3. In a preferred embodiment, the composition includes a MenA_{AH}-TT conjugate having 5 mcg MenA polysaccharide and ˜15 mcg TT; a MenC_{AH}-TT conjugate having 5 mcg MenC polysaccharide and ˜15 mcg TT; a MenW-TT conjugate having 5 mcg MenW polysaccharide and ˜7.5 mcg TT; and a MenY-TT conjugate having 5 mcg MenY polysaccharide and ˜6.5 mcg TT. The composition may further include Tris-HCl, sucrose, and sodium chloride.

In another embodiment, the composition includes a MenA_{AH}-TT conjugate; MenC_{AH}-TT conjugate; MenW-TT conjugate; and MenY-TT conjugate, which includes MenA polysaccharide; MenC polysaccharide; MenW polysaccharide; and MenY polysaccharide and TT carrier protein. The composition may further include sucrose and Trometanol. For example, in one embodiment, the composition includes 10 μg/mL MenA polysaccharide; 10 μg/mL MenC polysaccharide; 10 μg/mL MenW polysaccharide; and 10 μg/mL MenY polysaccharide; 88 μg/mL TT carrier protein; 164 mM sucrose; and 1.6 mM Trometanol.

In one embodiment, the composition has a total volume of about 0.5 ml. In one embodiment, a first dose of the composition has a total volume of about 0.5 ml. A “first dose” refers to the dose of the composition that is administered on Day 0. A “second dose” or “third dose” refers to the dose of the composition that is administered subsequently to the first dose, which may or may not be the same amount as the first dose.

In one aspect, the disclosure relates to a liquid immunogenic composition resulting from the lyophilized MenACWY-TT composition having been reconstituted with the liquid MnB bivalent rLP2086 composition. Reconstitution refers to restoring a dry lyophilized composition to a liquid form by the addition of a liquid diluent. In one preferred embodiment, the liquid MnB bivalent rLP2086 composition is not concomitantly administered, is not coadministered with, and is not simultaneously administered with the lyophilized MenACWY-TT composition, wherein the lyophilized MenACWY-TT composition has been reconstituted with a liquid composition that is not the liquid MnB bivalent rLP2086 composition. For example, in one preferred embodiment, the lyophilized MenACWY-TT composition is not reconstituted with an aqueous diluent consisting of sodium chloride and water and is not subsequently concomitantly administered, is not coadministered with, and is not simultaneously administered with the liquid MnB bivalent rLP2086 composition.

Rather, in a preferred embodiment, the lyophilized MenACWY-TT composition is administered with the MnB bivalent rLP2086 composition in one, i.e., a single, administration to the human. The resulting single administration (e.g., the MenABCWY composition) may result from the MnB bivalent rLP2086 composition, from a first container, being mixed with the lyophilized MenACWY-TT composition, from a second container. Alternatively, single administration of the MenABCWY composition may result from one (single) container that includes the MnB bivalent rLP2086 composition and the lyophilized MenACWY-TT composition. Delivery devices for vaccine or immunogenic compositions are known in the art. In one embodiment, the MenABCWY composition is administered concomitantly with any one of ibuprofen, paracetamol, and amoxicillin.

The composition is immunogenic after administration of a first dose to a human. In one embodiment, the first dose is about 0.5 ml in total volume.

The composition induces a bactericidal titer of serum immunoglobulin that is at least greater than 1-fold higher, preferably at least 2-fold higher, in the human after receiving the first dose than a bactericidal titer of serum immunoglobulin in the human prior to receiving the first dose, when measured under identical conditions in a serum bactericidal assay using human complement (hSBA).

The bactericidal titer or bactericidal immune response is against N. meningitidis serogroup B. In a preferred embodiment, the bactericidal titer or bactericidal immune response is against a N. meningitidis serogroup B fHBP subfamily A strain and against a N. meningitidis serogroup B fHBP subfamily B strain. Most preferably, the bactericidal titer or bactericidal immune response is at least against N. meningitidis serogroup B, fHBP subfamily B, B01 strain. In one embodiment, the composition induces a bactericidal titer of serum immunoglobulin that is at least greater than 1-fold, such as, for example, at least 1.01-fold, 1.1-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, or 16-fold higher in the human after receiving a dose of the composition than a bactericidal titer of serum immunoglobulin in the human prior to receiving said dose, when measured under identical conditions in a serum bactericidal assay using human complement.

In one embodiment, the composition is an immunogenic composition. In one embodiment, the composition is an immunogenic composition for a human. In another embodiment, the composition is a vaccine. A “vaccine” refers to a composition that includes an antigen, which contains at least one epitope that induces an immune response that is specific for that antigen. The vaccine may be administered directly into the subject by subcutaneous, oral, oronasal, or intranasal routes of administration. Preferably, the vaccine is administered intramuscularly. In one embodiment, the composition is a human vaccine. In one embodiment, the composition is an immunogenic composition against N. meningitidis.

In one embodiment, the composition is a liquid composition. In a preferred embodiment, the composition is a liquid suspension composition. In another preferred embodiment, the composition is not lyophilized.

### Stability

The terms “stable” and “stability” refer the ability of an antigen to remain immunogenic over a period of time. Stability may be measured in potency over time. The terms “stable” and “stability” further refer to the physical, chemical, and conformational stability of the immunogenic composition. Instability of a protein composition may be caused by chemical degradation or aggregation of the protein molecules to form higher order polymers, by dissociation of the heterodimers into monomers, deglycosylation, modification of glycosylation, or any other structural modification that reduces at least one biological activity of the protein composition included in the present disclosure. Stability may be assessed by methods well-known in the art, including measurement of a sample's light scattering, apparent attenuation of light (absorbance, or optical density), size (e.g. by size exclusion chromatography), in vitro or in vivo biological activity and/or properties by differential scanning calorimetry (DSC). Other methods for assessing stability are known in the art and can also be used according to the present disclosure.

In some embodiments, an antigen in a stable formulation of the disclosure may maintain at least 50%, 60%, 70%, 75%, 80%, 85%, 90%, 95%, 96%, 97%, 98%, 99% or 100% potency, as compared to a reference standard, for at least 1 month, 2 months, 3 months, 4 months, 5 months, 6 months, 9 months, 12 months, 18 months, 24 months, 30 months, 36 months, 42 months, 48 months, 54 months, or 60 months. In some embodiments, an antigen in a stable formulation of the disclosure may maintain at least 50% potency, as compared to a reference standard, for at least 1 year, 2 years, 3 years, 4 years or 5 years. The terms “stable” and “stability” also refer to the ability of an antigen to maintain epitopes or immunoreactivity over a period of time. For example, an antigen in a stable formulation of the disclosure may maintain at least 50%, 60%, 70%, 75%, 80%, 85%, 90%, 95%, 96%, 97%, 98%, 99% or 100% of its epitopes or immunoreactivity, as compared to a reference standard, for at least 1 month, 2 months, 3 months, 4 months, 5 months, 6 months, 9 months, 12 months, 18 months, 24 months, 30 months, 36 months, 42 months, 48 months, 54 months, or 60 months. In some embodiments, stability is measured with respect to an environmental condition. Non-limiting examples of environmental conditions include light, temperature, freeze/thaw cycles, agitation, and pH. One of skill in the art would be able to determine the presence of antigenic epitopes or immunoreactivity using the methods disclosed herein or other methods known in the art. In some embodiments, the stability of an antigen is measured from the date of its formulation. In some embodiments, the stability of an antigen is measured from the date of a change in its storage conditions. Non-limiting examples of changes in storage conditions include changing from frozen to refrigerated, changing from frozen to room temperature, changing from refrigerated to room temperature, changing from refrigerated to frozen, changing from room temperature to frozen, changing from room temperature to refrigerated, changing from light to dark, or introduction of agitation.

In one embodiment, the terms “stable” and “stability” includes the ability of an antigen to be bound to aluminum. For example, a stable formulation of the disclosure includes at least 50%, 60%, 70%, 75%, 80%, 85%, 90%, 95%, 96%, 97%, 98%, 99% or 100% of a protein that is bound to aluminum (e.g., aluminum phosphate) in the formulation, as compared to a reference standard, for at least 1 hour, 6 hours, 12 hours, 18 hours, 24 hours, 1 month, 2 months, 3 months, 4 months, 5 months, 6 months, 9 months, 12 months, 18 months, 24 months, 30 months, 36 months, 42 months, 48 months, 54 months, or 60 months. See, for example Example 13. In a preferred embodiment, at least 90%, more preferably at least 95%, and most preferably at least 99% of the total Subfamily A rLP2086 polypeptide (e.g., a polypeptide that includes the amino acid sequence set forth in SEQ ID NO: 1) is bound to aluminum in the composition. In a preferred embodiment, at least 90%, more preferably at least 95%, and most preferably at least 99% of the total Subfamily B rLP2086 polypeptide (e.g., a polypeptide that includes the amino acid sequence set forth in SEQ ID NO: 2) is bound to aluminum in the composition.

Determination of Aluminum Binding. A composition comprising aluminum and at least one protein antigen was centrifuged such that the aluminum was pelleted. Centrifugation of aluminum absorbed proteins is known in the art. See e.g., Egan et al., Vaccine, Vol. 27(24): 3175-3180 (2009). Aluminum-bound protein was also pelleted, while non-aluminum-bound protein remained in the supernatant. Total protein in the supernatant and pellet were determined by Lowry Assay. The percentage bound protein was calculated by dividing the total protein in the supernatant by the total protein added to the composition and multiplying by 100%. Similarly, the percentage unbound protein was calculated by dividing the total protein in the supernatant by the total protein added to the composition and multiplying by 100%. For compositions comprising both Subfamily A and Subfamily B antigens, the individual Subfamily A and B protein concentrations in the supernatant were determined by ion-exchange chromatography. The separation and elution of Subfamily A and B proteins was carried out using a strong anion column and a high salt concentration eluent. Both Subfamily A and B proteins were detected and quantified using a fluorescence detector set at Excitation=280 run and Emission=310 run. Subfamily A and Subfamily B proteins elute at distinct retention times and were quantified using a standard curve generated against a rLP2086 protein reference material. The percentage unbound protein was calculated by dividing the total protein in the supernatant by the total protein added to the composition and multiplying by 100%. The percentage bound protein was calculated by subtracting the percentage unbound protein from 100%.

### Polysorbate-80

Polysorbate 80 (PS-80) is a non-ionic surfactant. Accelerated stability studies using an in vitro monoclonal antibody based potency assay demonstrated instability of the subfamily B protein at higher molar ratios of PS-80 to MnB rLP2086 protein in the final formulation. Further experiments with varying ratios of PS-80 have demonstrated that the optimal molar ratio of PS-80 to MnB rLP2086 protein is approximately 2.8±1.4 to retain potency.

The concentration of PS-80 in the composition is dependent on a molar ratio of PS-80 to the polypeptide. In one embodiment, the composition includes a 2.8±1.4 molar ratio of PS-80 to the first polypeptide and to the second polypeptide. In one embodiment, the composition includes a 2.8±1.1 molar ratio of PS-80 to the first polypeptide and to the second polypeptide. In one embodiment, the composition includes at least 1.9, 2.0, 2.1, 2.2, 2.3, 2.4, 2.5, 2.6, 2.7, 2.8, 2.9, 3.0, 3.1, 3.2, or 3.3 molar ratio of PS-80 to polypeptide. In one embodiment, the composition includes at most 4.0, 3.9, 3.8, 3.7, 3.6, 3.5, 3.4, 3.3, 3.2, 3.1, 3.0, or 2.9 molar ratio of PS-80 to polypeptide. Any minimum value may be combined with any maximum value described herein to define a range. Preferably, the composition includes a 2.8 molar ratio of PS-80 to polypeptide.

The PS-80 to polypeptide molar ratio is determined by calculation from the measured concentration of PS-80 and the measured total polypeptide concentration, in which both values are expressed in moles. For example, PS-80 to Protein molar ratio is determined by calculation of the measured concentration of PS-80 (e.g., by reverse phase high pressure liquid chromatography (RP-HPLC)) to the measured total protein concentration (e.g., by ion exchange-high pressure liquid chromatography (IEX-HPLC)) in the final drug substance, where both values are expressed in moles.

A RP-HPLC is used to quantitate the concentration of Polysorbate 80 in vaccine formulations. The concentration of detergent is determined by saponification of the fatty acid moiety; Polysorbate 80 is converted to free oleic acid by alkaline hydrolysis at 40° C. The sample is separated by RP-HPLC using a C18 column and quantitated using a UV detector at a wavelength of 200 nm.

The first and the second polypeptides are resolved by anion-exchange HPLC. rLP2086(fHBP) Subfamily A and B proteins elute at distinct retention times and are quantitated using a standard curve generated against the respective rLP2086 protein reference material. The term “molar ratio” and a description of an immunogenic composition including a fHBP and PS-80 is further disclosed in WO2012025873 and US patent publication US 2013/0171194, which are each incorporated by reference in their entirety.

The term “molar ratio” as used herein refers to the ratio of the number of moles of two different elements in a composition. In some embodiments, the molar ratio is the ratio of moles of detergent to moles of polypeptide. In some embodiments, the molar ratio is the ratio of moles of PS-80 to moles of protein. In one embodiment, based on the protein and Polysorbate 80 concentrations, the Molar Ratio may be calculated using the following equation:

$$Molar Ratio = \frac{\% PS - 80}{mg / ml protein} \times 216$$

In one embodiment, the composition includes a molar ratio of PS-80 to MnB rLP2086 protein between 1.4 to 4.2 to retain potency. In one embodiment, the composition includes at least 1.2, 1.3, 1.4, 1.5, 1.6, 1.7, 1.8, 1.9, 2.0, 2.1, 2.2, 2.3, 2.4, 2.5, 2.6, 2.7, or 2.8. In one embodiment, the composition includes at most 4.2, 4.1, 4.0, 3.9, 3.8, 3.7, 3.7, 3.6, 3.5, 3.4, 3.3, 3.2, 3.1, 3.0, 2.9, or 2.8. Any minimum value may be combined with any maximum value described herein to define a range.

In one embodiment, the composition includes about 0.0015, 0.0017, 0.0019, 0.0021, 0.0023, 0.0025, 0.0027, 0.0029, 0.0031, 0.0033, 0.0035, 0.0037, 0.0039, 0.0041, 0.0043, 0.0045, 0.0047, 0.0049, 0.0051 mg/mL PS-80. Preferably, the composition includes about 0.0035 mg/mL PS-80.

In another embodiment, the composition includes at least 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, or 25 μg PS-80. In another embodiment, the composition includes at most 30 μg, 29 μg, 28 μg, 27 μg, 26 μg, 25 μg, 24 μg, 23 μg, 22 μg, 21 μg, 20 μg, 19 μg, or 18 μg PS-80. Any minimum value may be combined with any maximum value described herein to define a range. In a preferred embodiment, the composition includes at least 10 μg and at most 20 μg PS-80. In a most preferred embodiment, the composition includes about 18 μg PS-80.

In another embodiment, the composition includes a PS-80 concentration ranging from 0.0005% to 1%. For example, the PS-80 concentration in the composition may be at least 0.0005%, 0.005%, 0.01%, 0.02%, 0.03%, 0.04%, 0.05%, 0.06%, 0.07%, 0.08%, 0.09%, 0.10%, 0.2%, 0.3%, 0.4%, 0.5%, 0.6%, 0.7%, 0.8%, 0.9%, 1%, or 1.1% PS-80. In one embodiment, the PS-80 concentration in the composition may be at most 2.0%, 1.9%, 1.8%, 1.7%, 1.6%, 1.5%, 1.4%, 1.3%, 1.2%, 1.1%, 1.0%, 0.9%, 0.8%, or 0.7% PS-80. In a preferred embodiment, the composition includes about 0.07% PS-80. Any minimum value may be combined with any maximum value described herein to define a range.

In some embodiments, a composition that includes a combination of the first composition and the second composition may have a different molar ratio of polysorbate-80 in relation to the MnB rLP2086 polypeptides, as compared to the molar ratio of polysorbate-80 in relation to the MnB rLP2086 polypeptides in the first composition. In some embodiments, additional surfactant for the combined composition is not necessary to maintain solubility and stability of the MnB rLP2086 polypeptides in the combined composition. Accordingly, in one embodiment, the kit does not comprise greater than 0.02 mg polysorbate-80.

### Aluminum

The composition includes aluminum as aluminum phosphate. AlPO₄ is added as a stabilizer to provide enhanced manufacturability and stability. The process for producing an aluminum phosphate is described in US patent publication US 2009/0016946, which is incorporated by reference in its entirety. In one embodiment, the composition does not further include a multivalent cation, other than aluminum. In one embodiment, the composition does not further include Al(OH)₃ or Al(SO₄)₃.

In one embodiment, the composition includes at least 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, or 250 μg aluminum. In one embodiment, the composition includes at most 500 μg, 490 μg, 480 μg, 470 μg, 460 μg, 450 μg, 440 μg, 430 μg, 420 μg, 410 μg, 400 μg, 390 μg, 380 μg, 370 μg, 360 μg, 350 μg, 340 μg, 330 μg, 320 μg, 310 μg, 300 μg, 290 μg, 280 μg, 270 μg, 260 μg, or 250 μg aluminum. Any minimum value may be combined with any maximum value described herein to define a range. In a most preferred embodiment, the composition includes 250 μg aluminum.

In one embodiment, the composition includes at least 0.005 mg/ml, 0.01 mg/ml, 0.02 mg/ml, 0.03 mg/ml, 0.04 mg/ml, 0.05 mg/ml, 0.06 mg/ml, 0.07 mg/ml, 0.08 mg/ml, 0.09 mg/ml, 0.10 mg/ml, 0.2 mg/ml, 0.3 mg/ml, 0.4 mg/ml, or 0.5 mg/ml aluminum phosphate. In one embodiment, the composition includes at most 2.0 mg/ml, 1.9 mg/ml, 1.8 mg/ml, 1.7 mg/ml, 1.6 mg/ml, 1.5 mg/ml, 1.4 mg/ml, 1.3 mg/ml, 1.2 mg/ml, 1.1 mg/ml, 1.0 mg/ml, 0.9 mg/ml, 0.8 mg/ml, or 0.7 mg/ml PS-80. In a preferred embodiment, the composition includes about 0.07 mg/ml PS-80. Any minimum value may be combined with any maximum value described herein to define a range. In a preferred embodiment, the composition includes 0.5 mg/ml aluminum phosphate. In a most preferred embodiment, the composition includes 0.5 mg aluminum/ml as aluminum phosphate (AlPO₄). This concentration maintains binding (at least 90% binding or better) of the subfamily A and B proteins to aluminum.

In some embodiments, the combination of the first composition and the second composition changes the percentage of MnB rLP2086 polypeptides bound to the aluminum, when compared to the percentage of MnB rLP2086 polypeptides bound to the aluminum in the first composition. In some embodiments, the combination of the first and second compositions maintains binding of at least 90% of the total MnB rLP2086 polypeptides to the aluminum. Accordingly, in one embodiment, the percentage of total MnB rLP2086 polypeptides to the aluminum in the combined composition is at least 80%, 81%, 82%, 83%, 84%, 85%, 86%, 87%, 88%, 89%, 90%, 91%, 92%, 93%, 94%, 95%, 96%, 97%, 98%, 99%, or 100%. Preferably, the percentage of total MnB rLP2086 polypeptides to the aluminum in the combined composition is at least 90%, more preferably at least 95%, and most preferably at least 100%.

In another embodiment, the concentration of polypeptides bound to the aluminum in the immunogenic composition is not decreased after 24 hours, as compared to the concentration of polypeptides bound to the aluminum in the liquid composition prior to reconstituting the lyophilized composition. In another embodiment, the concentration of MenA_{AH}-TT conjugate in the immunogenic composition is not decreased after 24 hours, as compared to the concentration of the MenA_{AH}-TT conjugate in the lyophilized composition. In one embodiment, the concentration is decreased by at most 1%, 2%, 3%, 4%, 5%, 6%, 7%, 8%, 9%, 10%, 11%, 12%, 13%, 14%, 15%, 16%, 17%, 18%, 19%, or 20% after 24 hours, as compared to the respective concentration in the liquid composition prior to reconstitution.

In another embodiment, the concentration of MenC_{AH}-TT conjugate in the immunogenic composition is not decreased after 24 hours, as compared to the concentration of the MenC_{AH}-TT conjugate in the lyophilized composition. In another embodiment, the concentration of MenW-TT conjugate in the immunogenic composition is not decreased after 24 hours, as compared to the concentration of the MenW-TT conjugate in the lyophilized composition. In another embodiment, the concentration of MenY-TT conjugate in the immunogenic composition is not decreased after 24 hours, as compared to the concentration of the MenY-TT conjugate in the lyophilized composition. In one embodiment, the concentration is decreased by at most 1%, 2%, 3%, 4%, 5%, 6%, 7%, 8%, 9%, 10%, 11%, 12%, 13%, 14%, 15%, 16%, 17%, 18%, 19%, or 20% after 24 hours, as compared to the respective concentration in the lyophilized composition prior to reconstitution.

### Excipients

In one embodiment, the composition includes histidine. In one embodiment, the composition includes at least 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, or 850 μg of histidine. In one embodiment, the composition includes at most 1560 μg, 1500 μg, 1400 μg, 1300 μg, 1200 μg, 1100 μg, 1000 μg, 950 μg, 900 μg, 890 μg, 880 μg, 870 μg, 860 μg, 850 μg, 840 μg, 830 μg, 820 μg, 810 μg, 800 μg, 790 μg, or 780 μg of histidine. Any minimum value may be combined with any maximum value described herein to define a range. Preferably, the composition includes 780 μg histidine.

In one embodiment, the composition includes a tris, phosphate, or succinate buffer. In a preferred embodiment, the composition does not include tris buffer. In a preferred, the composition does not include phosphate buffer. In one preferred embodiment, the composition does not include succinate buffer. In a preferred embodiment, the composition includes histidine buffer.

In one embodiment, the composition includes sodium chloride. Sodium chloride concentration in MenABCWY composition may vary between 160.5-161.1 mM.

In one embodiment, the pH of the composition is between 5.5 and 7.5. In a preferred embodiment, the pH of the composition is between 5.8 and 7.0, most preferably pH 5.8 to pH 6.0. In one embodiment, the pH of the composition is at most 6.1. In one embodiment, the pH of the composition is 5.8.

### Kits

A further aspect of the disclosure is a kit for administering a dose of a composition for eliciting bactericidal antibodies against Neisseria meningitidis in a mammal.

In one aspect, the kit includes a first composition including a first polypeptide as described above and a second polypeptide as described above. In a preferred embodiment, the first polypeptide comprises the amino acid sequence set forth in SEQ ID NO: 1. In another preferred embodiment, the second polypeptide comprises the amino acid sequence set forth in SEQ ID NO: 2. The kit further includes a second composition including a MenA_{AH}-TT conjugate, a MenC_{AH}-TT conjugate, a MenW-TT conjugate, and a MenY-TT conjugate. In one embodiment, the kit includes at least two containers, wherein a first container includes the first composition, a second container includes the second composition.

In one embodiment, the kit includes a liquid first composition and a lyophilized second composition. Preferably, the kit includes a liquid MnB bivalent rLP2086 composition and a lyophilized MenACWY-TT composition.

In some embodiments, the composition includes a combination of the first composition and the second composition changes the molar ratio of polysorbate-80 in relation to the MnB rLP2086 polypeptides in the first composition. In some embodiments, additional surfactant for the combined composition is not necessary to maintain solubility and stability of the MnB rLP2086 polypeptides in the combined composition. Accordingly, in one embodiment, the kit does not comprise greater than 0.02 mg polysorbate-80.

In one embodiment of the disclosure, the kit does not further comprise any one of the following commercial immunogenic compositions: MENACTRA®, MENVEO®, ADACEL®, HAVRIX®, GARDASIL®, REPEVAX, or any combination thereof. For example, the kit preferably does not further include a meningococcal A, C, Y and W-135 polysaccharide conjugate (MCV4) composition, wherein the carrier protein is diphtheria toxoid. In one embodiment, the kit does not further include a meningococcal A, C, Y and W-135 polysaccharide conjugate (MCV4) composition, wherein the carrier protein is CRM₁₉₇. In one embodiment, the kit does not further comprise NIMENRIX vaccine, wherein NIMENRIX comprises a diluent consisting of sodium chloride and water.

### Bactericidal Activity

Disease incidence of MnB is approximately 1 in 100,000, meaning that extremely large numbers of subjects (400,000 to over 6 million) would be required to support a statistically significant assessment of efficacy. Thus, a serum bactericidal assay using human complement (hSBA), which is a surrogate of protection and vaccine efficacy, is used to assess immunogenicity in clinical trials.

Pfizer has built an extensive MnB strain collection (N=at least 1263) comprising IMD-causing isolates from Years 2000 to 2006. The MnB isolates were systematically collected from the US Centers for Disease Control and Prevention (CDC) and health and reference laboratories from European countries.

In one embodiment, immune response induced by administering the composition to a human is determined using a serum bactericidal assay using human complement (hSBA) against four N. meningitidis serogroup B (MnB) strains. The MnB strains used in the hSBA were selected from the strain pool. The strain pool represented a collection of systematically collected clinically relevant N. meningitidis strains.

The high proportion of hSBA response to all test strains, especially strains expressing lipoprotein 2086 variants with sequences heterologous to both the first polypeptide and the second polypeptide suggests that the composition is a broadly protective vaccine are sufficient to confer high seroprotection against N. meningitidis strains expressing rLP2086 (FHBP) from at least serogroup B, including additional serogroups, such as serogroup X.

### Subfamily A Strains

In one embodiment, the hSBA strain is an N. meningitidis strain that expresses LP2086 (fHBP) subfamily A protein. In one embodiment, the hSBA strain is an LP2086 (fHBP) subfamily A strain that expresses a lipoprotein 2086 variant that is heterologous to a N. meningitidis strain expressing A05. For example, in one embodiment, the hSBA strain is an LP2086 (fHBP) subfamily A strain that expresses a lipoprotein 2086 variant that is heterologous to strain M98250771.

In one embodiment, the hSBA strain is a N. meningitidis strain expressing fHBP A10. In one embodiment, the hSBA strain is a N. meningitidis strain expressing LP2086 (fHBP) A22. In one embodiment, the hSBA strain is a N. meningitidis strain expressing LP2086 (fHBP) A56. In a further embodiment, the hSBA strains are LP2086 (fHBP) A22 and LP2086 (fHBP) A56 strains. In another embodiment, the hSBA strain is a N. meningitidis strain expressing LP2086 A04. In one embodiment, the hSBA strain is a N. meningitidis strain expressing LP2086 A05. In one embodiment, the hSBA strain is a N. meningitidis strain expressing LP2086 A12. In one embodiment, the hSBA strain is a N. meningitidis strain expressing LP2086 A22. In one embodiment, the hSBA strain is a N. meningitidis strain expressing LP2086 A12. In one embodiment, the hSBA strain is a N. meningitidis strain expressing LP2086 A04. In one embodiment, the hSBA strain is a N. meningitidis strain expressing LP2086 A19. In one embodiment, the hSBA strain is a N. meningitidis strain expressing LP2086 A07. In a further embodiment, the hSBA strain includes any one of an A22-, A12-, A19-, A05-, and A07-expressing strain. In one embodiment, the hSBA strains include any one of an A06-, A15-, and A29-expressing strain.

In one embodiment, the immune response is bactericidal against a N. meningitidis serogroup B fHPB subfamily A strain that is heterologous to a N. meningitidis strain expressing A05. In one embodiment, the immune response is against N. meningitidis serogroup B A22 strain. In one embodiment, the immune response is against N. meningitidis serogroup B A56 strain. In one embodiment, the immune response is against N. meningitidis serogroup B A06 strain. In one embodiment, the immune response is against N. meningitidis serogroup B A15 strain. In one embodiment, the immune response is against N. meningitidis serogroup B A29 strain. In one embodiment, the immune response is against N. meningitidis serogroup B A62 strain. In one embodiment, the immune response is bactericidal against a N. meningitidis serogroup B subfamily A strain that is heterologous to N. meningitidis strain M98250771.

In one embodiment, the immune response is bactericidal against a N. meningitidis serogroup B subfamily A strain that expresses a factor H binding protein including an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the first polypeptide. In another embodiment, the immune response is bactericidal against a N. meningitidis serogroup B subfamily A strain that expresses a factor H binding protein including an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a factor H binding protein expressed by N. meningitidis strain M98250771. In a preferred embodiment, the immune response is bactericidal against a N. meningitidis serogroup B subfamily A strain that expresses a factor H binding protein including an amino acid sequence that has at least 80%, more preferably at least 84%, identity to a factor H binding protein expressed by N. meningitidis strain M98250771.

In another embodiment, the immune response is bactericidal against a N. meningitidis serogroup B subfamily A strain that expresses a factor H binding protein including an amino acid sequence that has at most 81%, 82%, 83%, 84%, 85%, 86%, 87%, 88%, 89%, 90%, 91%, 92%, 93%, 94%, 95%, 96%, 97%, 98%, 99%, or 100% identity to the first polypeptide. In another embodiment, the immune response is bactericidal against a N. meningitidis serogroup B subfamily A strain that expresses a factor H binding protein including an amino acid sequence that has at most 81%, 82%, 83%, 84%, 85%, 86%, 87%, 88%, 89%, 90%, 91%, 92%, 93%, 94%, 95%, 96%, 97%, 98%, 99%, or 100% identity to a factor H binding protein expressed by N. meningitidis strain M98250771. In a preferred embodiment, the immune response is bactericidal against a N. meningitidis serogroup B subfamily A strain that expresses a factor H binding protein including an amino acid sequence that has at most 85%, more preferably at most 99%, identity to a factor H binding protein expressed by N. meningitidis strain M98250771. Any minimum value may be combined with any maximum value described herein to define a range.

In one embodiment, the immune response elicited by the composition is bactericidal not only against a N. meningitidis serogroup B fHPB subfamily A strain but also a N. meningitidis strain expressing an fHBP subfamily A polypeptide, wherein the serogroup is not serogroup B. For example, in one preferred embodiment, the immune response elicited by the composition is bactericidal against a N. meningitidis serogroup B subfamily A strain and against a N. meningitidis serogroup C strain that expresses an fHBP subfamily A polypeptide heterologous to fHBP A05. For example, in one embodiment, the immune response is against a N. meningitidis serogroup C strain expressing fHBP A10. In another embodiment, the immune response is against a N. meningitidis serogroup W strain expressing fHBP A19. In one embodiment, the immune response is bactericidal against a N. meningitidis strain that expresses an fHBP subfamily A polypeptide, wherein the strain is heterologous to N. meningitidis strain M98250771.

### Subfamily B Strains

In one embodiment, the hSBA strain is an LP2086 (fHBP) subfamily B strain. In one embodiment, the hSBA strain is an LP2086 (fHBP) subfamily B strain that expresses a lipoprotein 2086 variant that is heterologous to a N. meningitidis strain expressing B01. For example, in one embodiment, the hSBA strain is an LP2086 (fHBP) subfamily B strain that expresses a lipoprotein 2086 variant that is heterologous to strain CDC1127. In a preferred embodiment, the hSBA strain is an LP2086 (fHBP) subfamily B strain that expresses a lipoprotein 2086 variant that is heterologous to strain CDC1573.

In one embodiment, the immune response is bactericidal against a N. meningitidis serogroup B fHPB subfamily B strain that is heterologous to a N. meningitidis strain expressing B01. In one embodiment, the immune response is against N. meningitidis serogroup B B24 strain. In one embodiment, the immune response is against N. meningitidis serogroup B B44 strain. In one embodiment, the immune response is against N. meningitidis serogroup B B16 strain. In one embodiment, the immune response is against N. meningitidis serogroup B B03 strain. In one embodiment, the immune response is against N. meningitidis serogroup B B09 strain. In one embodiment, the immune response is against N. meningitidis serogroup B B15 strain. In one embodiment, the immune response is against N. meningitidis serogroup B B153 strain. In one embodiment, the immune response is bactericidal against a N. meningitidis serogroup B subfamily B strain that is heterologous to N. meningitidis strain CDC1573.

In one embodiment, the immune response is bactericidal against a N. meningitidis serogroup B subfamily B strain that expresses a factor H binding protein including an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the second polypeptide. In another embodiment, the immune response is bactericidal against a N. meningitidis serogroup B subfamily B strain that expresses a factor H binding protein including an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a factor H binding protein expressed by N. meningitidis strain CDC1573. In a preferred embodiment, the immune response is bactericidal against a N. meningitidis serogroup B subfamily B strain that expresses a factor H binding protein including an amino acid sequence that has at least 80% identity, more preferably at least 87% identity, to a factor H binding protein expressed by N. meningitidis strain CDC1573. In another preferred embodiment, the immune response is bactericidal against a N. meningitidis serogroup B subfamily B strain that expresses a factor H binding protein including an amino acid sequence that has 100% identity to a factor H binding protein expressed by N. meningitidis strain CDC1573.

In another embodiment, the immune response is bactericidal against a N. meningitidis serogroup B subfamily B strain that expresses a factor H binding protein including an amino acid sequence that has at most 81%, 82%, 83%, 84%, 85%, 86%, 87%, 88%, 89%, 90%, 91%, 92%, 93%, 94%, 95%, 96%, 97%, 98%, 99%, or 100% identity to the second polypeptide. In another embodiment, the immune response is bactericidal against a N. meningitidis serogroup B subfamily B strain that expresses a factor H binding protein including an amino acid sequence that has at most 81%, 82%, 83%, 84%, 85%, 86%, 87%, 88%, 89%, 90%, 91%, 92%, 93%, 94%, 95%, 96%, 97%, 98%, 99%, or 100% identity to a factor H binding protein expressed by N. meningitidis strain CDC1573. In a preferred embodiment, the immune response is bactericidal against a N. meningitidis serogroup B subfamily B strain that expresses a factor H binding protein including an amino acid sequence that has at most 88% identity, more preferably at least 99% identity, to a factor H binding protein expressed by N. meningitidis strain CDC1573. Any minimum value may be combined with any maximum value described herein to define a range.

In one embodiment, the hSBA strain is an LP2086 (fHBP) B24 strain. In another embodiment, the hSBA strains is an LP2086 (fHBP) B44 strain. In a further embodiment, the hSBA strains includes LP2086 (fHBP) B24 and LP2086 (fHBP) B44 strains. In one embodiment, the hSBA strains includes LP2086 (fHBP) A22, LP2086 (fHBP) A56, LP2086 (fHBP) B24, and LP2086 (fHBP) B44 strains. In one embodiment, the hSBA strain includes B15. In one embodiment, the hSBA strain includes B153. In another embodiment, the hSBA strain is an LP2086 B16 strain. In one embodiment, the hSBA strain is an LP2086 B03 strain. In one embodiment, the hSBA strain is an LP2086 B09 strain. In a further embodiment, the hSBA strains include B24, B16, B44, B03, and B09, or any combination thereof. In another embodiment, the hSBA strains include B24, B16, B44, A22, B03, B09, A12, A19, A05, and A07, or any combination thereof. In another embodiment, the hSBA strains include A06, A07, A12, A15, A19, A29, B03, B09, B15, and B16, or any combination thereof.

In one embodiment, the method induces an immune response against a N. meningitidis serogroup B fHPB subfamily A strain and against a N. meningitidis serogroup B fHPB subfamily B strain. Preferably, the immune response is bactericidal against a N. meningitidis serogroup B fHPB subfamily A strain and against a N. meningitidis serogroup B fHPB subfamily B strain.

In one embodiment, the immune response elicited by the composition is bactericidal not only against a N. meningitidis serogroup B fHPB subfamily B strain but also a N. meningitidis strain expressing an fHBP subfamily B polypeptide, wherein the serogroup is not serogroup B. For example, in one preferred embodiment, the immune response elicited by the composition is bactericidal against a N. meningitidis serogroup B subfamily B strain and against a N. meningitidis serogroup Y strain that expresses an fHBP subfamily B polypeptide heterologous to fHBP B01. For example, in one embodiment, the immune response is against a N. meningitidis serogroup A strain expressing fHBP B16. In another embodiment, the immune response is against a N. meningitidis serogroup Y strain expressing fHBP B47. In another embodiment, the immune response is against a N. meningitidis serogroup X strain expressing fHBP B49. In one embodiment, the immune response is bactericidal against a N. meningitidis strain that expresses an fHBP subfamily B polypeptide, wherein the strain is heterologous to N. meningitidis serogroup B strain CDC1573.

In one aspect, the invention relates to uses of a composition including a first lipidated polypeptide variant of a Neisseria meningitidis serogroup B factor H binding protein (fHBP) and a second lipidated polypeptide variant of a Neisseria meningitidis serogroup B fHBP. In one embodiment, the composition induces a bactericidal immune response against at least one N. meningitidis serogroup B strain expressing a polypeptide selected from the group consisting of B A02, A28, A42, A63, A76, B05, B07, B08, B13, B52 and B107. For example, in one aspect, the invention relates to uses of a composition including a first lipidated polypeptide including the amino acid sequence set forth in SEQ ID NO: 1 and a second lipidated polypeptide including the amino acid sequence set forth in SEQ ID NO: 2.

In one embodiment, the hSBA strain is a N. meningitidis strain expressing fHBP B05. In one embodiment, the hSBA strain is a N. meningitidis strain expressing LP2086 (fHBP) B07. In one embodiment, the hSBA strain is a N. meningitidis strain expressing LP2086 (fHBP) B08. In another embodiment, the hSBA strain is a N. meningitidis strain expressing LP2086 B13. In one embodiment, the hSBA strain is a N. meningitidis strain expressing LP2086 B52. In one embodiment, the hSBA strain is a N. meningitidis strain expressing LP2086 B107. In a further embodiment, the hSBA strain includes any one strain selected from the group consisting of B05, B07, B08, B13, B52 and B107. In a further embodiment, the hSBA strain includes any one strain selected from the group consisting of B05, B07, B08, B13, B52, B107, B01, B24, B44, B16, B03, B09, B15, and B153.

In one embodiment, the immune response is bactericidal against a N. meningitidis serogroup B subfamily B strain that is heterologous to a N. meningitidis strain expressing B01. In one embodiment, the immune response is against N. meningitidis serogroup B B05 strain. In one embodiment, the immune response is against N. meningitidis serogroup B B07 strain. In one embodiment, the immune response is against N. meningitidis serogroup B B08 strain. In one embodiment, the immune response is against N. meningitidis serogroup B B13 strain. In one embodiment, the immune response is against N. meningitidis serogroup B B52 strain. In one embodiment, the immune response is against N. meningitidis serogroup B B107 strain. In one embodiment, the immune response is against N. meningitidis serogroup B B24 strain. In one embodiment, the immune response is against N. meningitidis serogroup B B44 strain. In one embodiment, the immune response is against N. meningitidis serogroup B B16 strain. In one embodiment, the immune response is against N. meningitidis serogroup B B03 strain. In one embodiment, the immune response is against N. meningitidis serogroup B B09 strain. In one embodiment, the immune response is against N. meningitidis serogroup B B15 strain. In one embodiment, the immune response is against N. meningitidis serogroup B B153 strain. In one embodiment, the immune response is bactericidal against a N. meningitidis serogroup B subfamily B strain that is heterologous to N. meningitidis strain CDC1573.

In one embodiment, the immune response is against a N. meningitidis serogroup B strain selected from the group consisting of A02, A28, A42, A63, and A76. In one embodiment, the immune response is against a N. meningitidis serogroup B strain selected from the group consisting of B05, B07, B08, B13, B52, B107, B01, B24, B44, B16, B03, B09, B15, and B153, and any combination thereof.

In one embodiment, the hSBA strains include B05, B07, B08, B13, B52 and B107, and any combination thereof. In a further embodiment, the hSBA strains include B05, B07, B08, B13, B52 and B107, B24, B16, B44, B03, and B09, and any combination thereof. In one embodiment, the hSBA strains include A02, A28, A42, A63, A76, B05, B07, B08, B13, B52 and B107, and any combination thereof. In another embodiment, the hSBA strains further include A06, A07, A12, A15, A19, A29, B03, B09, B15, and B16, or any combination thereof. In another embodiment, the hSBA strains include A02, A28, A42, A63, A76, B05, B07, B08, B13, B52 and B107, A06, A07, A12, A15, A19, A29, B03, B09, B15, and B16, and any combination thereof.

In one embodiment, the method induces an immune response against a N. meningitidis serogroup B subfamily A strain and against a N. meningitidis serogroup B subfamily B strain. Preferably, the immune response is bactericidal against a N. meningitidis serogroup B subfamily A strain and against a N. meningitidis serogroup B subfamily B strain. In one embodiment, the method induces an immune response against a N. meningitidis serogroup B strain selected from the group consisting of A02, A28, A42, A63, A76, B05, B07, B08, B13, B52 and B107, and any combination thereof. In one embodiment, the method induces an immune response against a N. meningitidis serogroup B strain selected from the group consisting of A02, A28, A42, A63, A76, B05, B07, B08, B13, B52 and B107, A06, A07, A12, A15, A19, A29, B03, B09, B15, and B16, and any combination thereof.

### Titers

In one embodiment, the composition induces an increase in bactericidal titer in the human, as compared to the bactericidal titer in the human prior to administration of a dose of the composition, when measured under identical conditions in an hSBA. In one embodiment, the increase in bactericidal titer is compared to the bactericidal titer in the human before administration of the first dose of the composition, as compared to the bactericidal titer in the human prior to administration of the first dose of the composition, when measured under identical conditions in an hSBA. In one embodiment, the increase in titer is observed after a second dose of the composition, as compared to the bactericidal titer in the human prior to administration of the second dose of the composition, when measured under identical conditions in an hSBA. In another embodiment, the increase in bactericidal titer is observed after a third dose of the composition, as compared to the bactericidal titer in the human prior to administration of the third dose of the composition, when measured under identical conditions in an hSBA.

In one embodiment, the composition induces a bactericidal titer in the human after administration of a dose, wherein the bactericidal titer is at least greater than 1-fold higher than the bactericidal titer in the human prior to administration of the dose, when measured under identical conditions in an hSBA. For example, the bactericidal titer may be at least 1.01-fold, 1.1-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, or 16-fold higher in the human after receiving a dose of the composition, as compared to the bactericidal titer in the human prior to administration of the dose, when measured under identical conditions in an hSBA.

In one embodiment, a “responder” refers to a human, wherein the composition induces a bactericidal titer in the human after administration of a dose, wherein the bactericidal titer is at least greater than 1-fold higher than the bactericidal titer in the human prior to administration of the dose. In a preferred embodiment, the responder achieves at least a 4-fold rise in hSBA titer, as compared to a bactericidal titer in the human prior to administration of the dose. Such a responder may be referred to as having a protective titer. In some embodiments, a protective titer is one that is greater than 1:4.

In one embodiment, the hSBA titer is the reciprocal of the highest dilution of a serum sample that produces a measurable effect. For example, in one embodiment, the hSBA titer is the reciprocal of the highest 2-fold dilution of a test serum that results in at least a 50% reduction of MnB bacteria (50% bacterial survival) compared to the T30 CFU value (i.e., the number of bacteria surviving after incubation in assay wells containing all assay components except test serum; 100% bacterial survival).

In one embodiment, the composition induces a bactericidal titer in the human after receiving the first dose that is at least 2-fold higher than the bactericidal titer in the human prior to receiving the first dose (e.g., higher than the bactericidal titer in the human in the absence of the first dose), when measured under identical conditions in the hSBA. In one embodiment, the composition induces a bactericidal titer in the human that is at least 4-fold higher than the bactericidal titer in the human prior to receiving the first dose, when measured under identical conditions in a human serum bactericidal assay that utilizes human complement (hSBA). In one embodiment, the composition induces a bactericidal titer in the human that is at least 8-fold higher than the bactericidal titer in the human prior to receiving the first dose, when measured under identical conditions in a human serum bactericidal assay that utilizes human complement (hSBA).

In a preferred embodiment, the human serum complement is derived from a human having low intrinsic bactericidal activity for a given hSBA test strain. Low intrinsic bactericidal activity refers to, for example, a bactericidal titer that is at least less than a 1:4 dilution against the given hSBA test strain. In one embodiment, the human complement is derived from a human having an hSBA titer that is at least less than 1:4, such as a 1:2 dilution, against the given hSBA test strain, wherein the composition was not administered to the human.

A human may exhibit an hSBA titer of less than 1:4 prior to administration of a composition, such as the bivalent rLP2086 composition, or a human may exhibit an hSBA titer of ≥1:4 prior to administration of the composition. Accordingly, in preferred embodiments and examples, administration of at least one dose of the composition to the human results in an hSBA titer that is at least 4-fold greater than the titer in the human prior to the administration. In some embodiments, administration of at least one dose of the composition to the human results in an hSBA titer that is at least greater than 1:4, such as, for example, an hSBA titer of ≥1:8, an hSBA titer of 1:16, and an hSBA titer of ≥1:32. The respective Examples described herein include assessments of the proportion of human subjects having an hSBA titer ≥21:8 and/or ≥1:16, wherein the bivalent rLP2086 composition was administered to the human. In some embodiments, a 4-fold rise in titer in the human after administration of the composition as compared to before administration of the composition show that protection is associated with the composition. In some embodiments, such preferred assessments of hSBA titers greater than 1:4 show that the protection, i.e., the bactericidal immune response induced in the human, is associated with the composition.

In one embodiment, the human has an hSBA titer equal to or greater than the hSBA's lower limit of quantitation (LLOQ) after administration of the first dose of the composition. In another embodiment, the human has an hSBA titer equal to or greater than the hSBA's LLOQ after administration of the second dose of the composition. In another embodiment, the human has an hSBA titer equal to or greater than the hSBA's LLOQ after administration of the third dose of the composition.

### Methods and Administration

In one aspect, the disclosure relates to a method of inducing an immune response against N. meningitidis in a human. In another aspect, the disclosure relates to a method of vaccinating a human.

In some embodiments, the method includes administering to the human the composition, wherein the composition induces an immune response to each of N. meningitidis serogroups A, C, W-135 and Y capsular polysaccharides, wherein the immune response includes a titer of serum bactericidal antibodies, and wherein the titer is higher than that induced by a licensed vaccine against N. meningitidis serogroups A, C, W-135 and Y meningococcal capsular polysaccharides. In some embodiments, the licensed vaccine against N. meningitidis serogroups A, C, W-135 and Y meningococcal capsular polysaccharides is MENVEO.

In some embodiments, the method includes administering to the human the composition, wherein the composition induces an immune response to N. meningitidis serogroup B, wherein the immune response includes a titer of serum bactericidal antibodies that is higher than a titer of serum bactericidal antibodies induced by a licensed vaccine against N. meningitidis serogroup B. In some embodiments, the licensed vaccine against N. meningitidis serogroup B is TRUMENBA.

In some embodiments, the method includes administering to the human the composition, wherein the composition induces an immune response to each of N. meningitidis serogroups A, C, W-135 and Y capsular polysaccharide and N. meningitidis serogroup B, wherein the immune response includes a titer of serum bactericidal antibodies to each of N. meningitidis serogroups A, C, W-135 and Y capsular polysaccharide that is higher than a titer of serum bactericidal antibodies induced by a licensed vaccine against N. meningitidis serogroups A, C, W-135 and Y capsular polysaccharide, and wherein the immune response includes a titer of serum bactericidal antibodies to N. meningitidis serogroup B that is higher than a titer of serum bactericidal antibodies induced by a licensed vaccine against N. meningitidis serogroup B. In some embodiments, the licensed vaccine against N. meningitidis serogroups A, C, W-135 and Y meningococcal capsular polysaccharides is MENVEO. In some embodiments, the licensed vaccine against N. meningitidis serogroup B is TRUMENBA.

The disclosure relates to a method for eliciting an immune response in a human of any age. In some embodiments, the human is aged at least 4 weeks, 5 weeks, 6 weeks, 7 weeks, 8 weeks, 9 weeks, 10 weeks, 11 weeks, or 12 weeks old. For example, in a preferred embodiment, the human is aged at least 6 weeks. As is known in the art, a Meningococcal Group A, C, W-135, and Y Conjugate Vaccine, such as NIMENRIX®, is suitable for infants as early as six weeks of age, and can be administered to any human aged six weeks and above. In some embodiments, the human is aged at least 6 months, 7 months, 8 months, 9 months, 10 months, 11 months, or 12 months. For example, in a preferred embodiment, the human is aged at least 12 months. In one embodiment, the human is aged between 12 and 18 months. In another aspect, the disclosure relates to a method for eliciting an immune response in a patient aged at least 18 months. In one embodiment, the human is aged between 18 and 24 months. In yet another aspect, the disclosure relates to a method for eliciting an immune response in a patient aged at least 24 months. In one embodiment, the human is aged between 24 months and 10 years. In another aspect, the disclosure relates to a method for eliciting an immune response in a patient aged 10 years and above.

In some embodiments, the human is aged between 10 years and 25 years. In some embodiments, the human is aged between 10 to 26 years old. In some embodiments, the human is aged between 12 to <18 Months. In some embodiments, the human is aged between 18 to <24 Months. In some embodiments, the human is aged between 18 to <24 Months. In some embodiments, the human is aged between ≥24 Months to <10 Years.

In some embodiments, the human is at least 16 years old. In such embodiments, the method includes administering one dose to the human, preferably at most one dose to the human aged at least 16 years old. In some embodiments, the human is at most 17 years of age.

In some embodiments, the human is aged between 10 to 12 years old. In such embodiments, the method includes administering at least one dose to the human. In a preferred embodiment, a second dose is administered to the human about 6 months after the first dose. In a preferred embodiment, the method includes administering a first dose and a second dose of the composition to the human aged between 10 to 12 years old, and a third dose of the composition is administered to the human at least four years after the first dose.

In some embodiments, the method includes administering at least two doses to the human. In a preferred embodiment, the two doses are at least about 6 months apart. In a preferred embodiment, the method includes administering a first dose and a second dose of the composition to the human aged between 10 to 12 years old, and a third dose of the composition is administered to the human at least four years after the first dose.

In some embodiments, the method includes administering a first dose of the composition to the human at about 11 years old and administering at least two doses of the composition to the human at least four years after the first dose. In some embodiments, the method includes administering the second and subsequent doses of the composition about five years after the first dose.

In some embodiments, the human is seronegative against N. meningitidis serogroup A. In some embodiments, the human is seronegative against N. meningitidis serogroup C. In some embodiments, the human is seronegative against N. meningitidis serogroup W. In some embodiments, the human is seronegative against N. meningitidis serogroup Y. In some embodiments, the human is seronegative against N. meningitidis serogroup A, C, W-135 and Y capsular polysaccharides.

In some embodiments, the human is seropositive against N. meningitidis serogroup A. In some embodiments, the human is seropositive against N. meningitidis serogroup C. In some embodiments, the human is seropositive against N. meningitidis serogroup W. In some embodiments, the human is seropositive against N. meningitidis serogroup Y. In some embodiments, the human is seropositive against N. meningitidis serogroup A, C, W-135 and Y capsular polysaccharides.

In one embodiment, the method includes administering to the human at least one dose of the composition described above. In a preferred embodiment, the method includes administering to the human at most one dose of the composition described above. In another embodiment, the method includes administering to the human at least a first dose and a second dose of the composition described above.

In one embodiment, the second dose is administered at least 20, 30, 50, 60, 100, 120, 160, 170, or 180 days after the first dose, and at most 250, 210, 200, or 190 days after the first dose. Any minimum value may be combined with any maximum value described herein to define a range.

In another embodiment, the second dose is administered about 30 days after the first dose. In another embodiment, the second dose is administered about 60 days after the first dose, such as, for example, in a 0, 2 month immunization schedule. In another embodiment, the second dose is administered about 180 days after the first dose, such as, for example, in a 0, 6 month immunization schedule. In yet another embodiment, the second dose is administered about 120 days after the first dose, such as, for example, in a 2, 6 month immunization schedule.

In one embodiment, the method includes administering to the human two doses of the composition and at most two doses. In one embodiment, the two doses are administered within a period of about 6 months after the first dose. In one embodiment, the method does not include further administration of a booster to the human. A “booster” as used herein refers to an additional administration of the composition to the human. Administering to the human at most two doses of the composition may be advantageous. Such advantages include, for example, facilitating a human to comply with a complete administration schedule and facilitating cost-effectiveness of the schedule.

In one embodiment, the first dose and the second dose are administered to the human over a period of about 25, 30, 40, 50, 60, 70, 80, 90, 100, 110, 120, 130, 140, 150, 160, 170, 180, 190, or 200 days, and most 400, 390, 380, 370, 365, 350, 340, 330, 320, 310, 300, 290, 280, 270, 260, 250, 240, 230, 220, 210, or 200 days after the first dose. Any minimum value may be combined with any maximum value described herein to define a range. Preferably, the first and second doses will be administered at least 4 weeks apart e.g. ≥8 weeks apart, ≥2 months apart, ≥3 months apart, ≅6 months apart, etc.

In one embodiment, the first dose and the second dose are administered to the human over a period of about 30 days. In another embodiment, the first dose and the second dose are administered to the human over a period of about 60 days. In another embodiment, the first dose and the second dose are administered to the human over a period of about 180 days.

Conveniently, the first dose can be administered at substantially the same time as (e.g. during the same medical consultation or visit to a healthcare professional or within 24 hours of the first dose of the meningococcal vaccine) another vaccine e.g. at substantially the same time as a hepatitis B virus vaccine, a diphtheria vaccine, a tetanus vaccine, a pertussis vaccine (either cellular or, preferably, acellular), a Haemophilus influenzae type b vaccine, a Streptococcus pneumoniae vaccine, and/or a polio vaccine (preferably in inactivated poliovirus vaccine). Each of these optionally co-administered vaccines may be a monovalent vaccine or may be part of a combination vaccine (e.g. as part of a DTP vaccine).

Conveniently, the second dose can be administered at substantially the same time as (e.g. during the same medical consultation or visit to a healthcare professional or within 24 hours of the second dose of the meningococcal vaccine) another vaccine e.g. at substantially the same time as a hepatitis B virus vaccine, a diphtheria vaccine, a tetanus vaccine, a pertussis vaccine (either cellular or acellular), a Haemophilus influenzae type b vaccine, a Streptococcus pneumoniae vaccine, a polio vaccine (preferably in inactivated poliovirus vaccine), an influenza vaccine, a chickenpox vaccine, a measles vaccine, a mumps vaccine, and/or a rubella vaccine. Each of these optionally co-administered vaccines may be a monovalent vaccine or may be part of a combination vaccine (e.g. as part of an MMR vaccine).

Conveniently, the third dose can be administered at substantially the same time as (e.g. during the same medical consultation or visit to a healthcare professional or within 24 hours of the third dose of the meningococcal vaccine) another vaccine e.g. at substantially the same time as a hepatitis B virus vaccine, a diphtheria vaccine, a tetanus vaccine, a pertussis vaccine (either cellular or acellular), a Haemophilus influenzae type b vaccine, a Streptococcus pneumoniae vaccine, a polio vaccine (preferably in inactivated poliovirus vaccine), an influenza vaccine, a chickenpox vaccine, a measles vaccine, a mumps vaccine, and/or a rubella vaccine. Each of these optionally co-administered vaccines may be a monovalent vaccine or may be part of a combination vaccine (e.g. as part of an MMR vaccine).

In one embodiment, a three-dose schedule of the composition induces a bactericidal titer against multiple strains expressing LP2086 (fHBP) heterologous to the first and/or second polypeptide in a greater percentage of humans than a two-dose schedule.

In one embodiment, the method includes administering to the human three doses of the composition. In another embodiment, the method includes administering at most three doses of the composition. In one embodiment, the three doses are administered within a period of about 6 months after the first dose. In one embodiment, the method includes an administration of a booster dose to the human after the third dose. In another embodiment, the method does not include administration of a booster dose to the human after the third dose. In another embodiment, the method does not further include administering a fourth or booster dose of the composition to the human. In a further embodiment, at most three doses within a period of about 6 months are administered to the human.

In an exemplary embodiment, the second dose is administered about 30 days after the first dose, and the third dose is administered about 150 days after the second dose, such as, for example, in a 0, 1, 6 month immunization schedule. In another exemplary embodiment, the second dose is administered about 60 days after the first dose, and the third dose is administered about 120 days after the second dose, such as, for example, in a 0, 2, 6 month immunization schedule.

In one embodiment, the first dose, second dose, and third dose are administered to the human over a period of about 150, 160, 170, or 180 days, and at most 240, 210 200, or 190 days. Any minimum value may be combined with any maximum value described herein to define a range. Preferably, the first dose, second dose, and third dose is administered to the human over a period of about 180 days or 6 months. For example, the second dose may be administered to the human about 60 days after the first dose, and the third dose may be administered to the human about 120 days after the second dose. Accordingly, an exemplary schedule of administration includes administering a dose to the human at about months 0, 2, and 6.

As described above, multiple doses of the immunogenic composition may be administered to the human, and the number of days between each dose may vary. An advantage of the method includes, for example, flexibility for a human to comply with the administration schedules.

In one embodiment, the method includes administering to the human at most three doses of the identical immunogenic composition. For example, in a preferred embodiment, the method does not include administering to the human a first dose of a first composition, administering to the human a second dose of a second composition, and administering to the human a third dose of a third composition, wherein the first, second, and third compositions are not identical. In another embodiment, the method includes administering to the human at most four doses of the identical immunogenic composition.

### EXAMPLES

The following Examples illustrate embodiments of the disclosure. Unless noted otherwise herein, reference is made in the following Examples to a MnB bivalent rLP2086 composition, at the 120-μg bivalent rLP2086 dose level, which is a preferred exemplary embodiment of a composition including: 60 μg of a first lipidated polypeptide including the amino acid sequence set forth in SEQ ID NO: 1 per 0.5 mL dose, 60 μg of a second lipidated polypeptide including the amino acid sequence set forth in SEQ ID NO: 2 per 0.5 mL dose, 2.8 molar ratio polysorbate-80 to the first polypeptide, 2.8 molar ratio polysorbate-80 to the second polypeptide, 0.5 mg Al³⁺/ml of the composition, 10 mM histidine, and 150 mM sodium chloride.

More specifically, the investigational bivalent recombinant rLP2086 vaccine at the 120-g bivalent rLP2086 dose level includes (a) 60 μg of a first lipidated polypeptide including the amino acid sequence set forth in SEQ ID NO: 1; (b) 60 μg of a second lipidated polypeptide including the amino acid sequence set forth in SEQ ID NO: 2; (c) 18 μg polysorbate-80; (d) 250 μg aluminum; (e) 780 μg histidine, and (f) 4380 μg sodium chloride. Each dose is 0.5 mL.

Unless noted otherwise herein, reference is made in the following Examples to a MenACWY-TT composition, which is a preferred exemplary embodiment of a tetravalent meningococcal polysaccharide conjugated composition that includes Neisseria meningitidis capsular polysaccharides A, C, W-135 and Y each coupled to tetanus toxoid as a carrier protein. The Neisseria meningitidis serogroups A and C polysaccharides are conjugated with an adipic dihydrazide (AH) spacer and indirectly conjugated to the tetanus toxoid whereas the W-135 and Y polysaccharides are conjugated directly to tetanus toxoid. The composition does not contain any preservatives or adjuvants.

More specifically, the lyophilized MenACWY-TT composition described in the examples below includes 5 micrograms of Neisseria meningitidis serogroup A polysaccharide conjugated to tetanus toxoid carrier protein; 5 micrograms of Neisseria meningitidis serogroup C polysaccharide conjugated to tetanus toxoid carrier protein; 5 micrograms of Neisseria meningitidis serogroup W-135 polysaccharide conjugated to tetanus toxoid carrier protein; 5 micrograms of Neisseria meningitidis serogroup Y polysaccharide conjugated to tetanus toxoid carrier protein; 28 mg sucrose; 97 μg trometamol, per dose (0.5 mL).

### Example 1: The MenABCWY Composition

The final MenABCWY composition is prepared by reconstituting the lyophilized MenACWY-TT Drug Product (described in Example 2 below) vial with 0.67 mL of MnB Bivalent rLP2086 Drug Product (described in Example 3 below) in order to withdraw 0.5 mL dose of MenABCWY vaccine for intramuscular injection. All components used in the preparation of the MenABCWY vaccine and their functions are provided in Table 1 below.

TABLE 1
Composition of MenABCWY vaccine

| Ingredients | Amount/dose | |
|---|---|---|
| MnB rLP2086 subfamily A (SEQ ID NO: 1) | 60 | mcg |
| MnB rLP2086 subfamily B (SEQ ID NO: 2) | 60 | mcg |
| MenA_{AH}-TT conjugate | 5 | mcg MenA |
| (mean TT/polysaccharide ratio: ~3) | ~7.5 | mcg TT |
| MenC_{AH}-TT conjugate | 5 | mcg MenC |
| (mean TT/polysaccharide ratio: ~3) | ~7.5 | mcg TT |
| MenW-TT conjugate | 5 | mcg MenW |
| (mean TT/polysaccharide ratio: ~1.5) | ~3.75 | mcg TT |
| MenY-TT conjugate | 5 | mcg MenY |
| (mean TT/polysaccharide ratio: ~1.3) | ~3.25 | mcg TT |
| Tris-HCl | 97 | mcg |
| Sodium Chloride^{a} | 4.69-4.71 | mg |
| Sucrose | 28 | mg |
| L-Histidine | 0.78 | mg |
| Polysorbate 80 (PS80) | 0.02 | mg |
| Aluminum phosphate | 0.25 | mg aluminum |

| | |
|---|---|
| Water for injection | qs to 0.5 mL |

^{a}Sodium chloride concentration in MenABCWY Vaccine may vary between 160.5-161.1 mM based on the composition of the clinical and commercial NIMENRIX Drug Product (DP) lots.

MenABCWY is a combination product comprising of a single-dose of lyophilized MenACWY-TT in a 2 mL Type 1 glass vial, a non-graduated 1 mL Type I glass standard pre-filled syringe (PFS) containing MnB Bivalent rLP2086 suspension for reconstitution, and a 13 mm vial adapter.

The lyophilized MenACWY-TT drug product will be reconstituted by affixing the supplied vial adapter to the lyophilized vial and attaching the MnB Bivalent rLP2086 PFS to the vial adapter. This process is intended to facilitate aseptic delivery of the MnB suspension from the PFS into the vial and subsequent transfer of the reconstituted vaccine from the vial into the PFS for administration. After reconstitution, the contents of the vial will be withdrawn into the same syringe via the vial adapter. The vial adapter will be detached from the syringe and a needle will be affixed to the PFS for intramuscular (IM) injection. MnB Bivalent rLP2086 PFS will be designed to have a higher fill volume than TRUMENBA (˜0.67 mL as opposed to the current 0.57 mL) to ensure that target dose volume (0.5 mL) of the final vaccine (MenABCWY) can be delivered.

The final vaccine composition includes rLP2086 subfamily A and B proteins formulated at 120 μg/mL/subfamily, purified capsular polysaccharides of Neisseria meningitidis serogroups A, C, W and Y at concentration of 10 μg/mL/type conjugated to tetanus toxoid at ratios of ˜1:3, ˜1:3, ˜1:1.5, ˜1:1.3, respectively, in 10 mM histidine, 1.2 mM Tris buffer, 160.5-161.1 mM sodium chloride, 0.5 mg/mL aluminum as aluminum phosphate (AlPO4), 1.35 mg/mL polysorbate 80 and 56 mg/mL sucrose

### Example 2: Description and Composition of the MnB Bivalent rLP2086 Drug Product

MnB bivalent rLP2086 drug product is a sterile liquid formulation composed of rLP2086 subfamily A and B proteins formulated at 120 μg/mL/subfamily in 10 mM histidine buffer, 150 mM sodium chloride (NaCl) at pH 6.0 with 0.5 mg/mL aluminum as aluminum phosphate (AlPO4). Polysorbate 80 (PS-80) is added to drug substance to obtain the target PS-80 to protein molar ratio. Therefore, PS-80 is not added during the drug product formulation but is present in the final drug product at the same ratio. The drug product is filled into 1 mL syringes. A single dose of vaccine is 0.5 mL with no preservatives.

TABLE 2
Composition of MnB Bivalent rLP2086 Drug Product

| Ingredients | Quantity/dose | |
|---|---|---|
| MnB rLP2086 subfamily A (SEQ ID NO: 1) | 120 | μg/mL |
| MnB rLP2086 subfamily B (SEQ ID NO: 2) | 120 | μg/mL |
| Sodium chloride | 150 | mM |
| L-Histidine | 10 | mM |
| Aluminum phosphate | 0.50 | mg Aluminum |
| | | phosphate/mL |

| | |
|---|---|
| Water for injection | qs to 1 mL |

^{a} Polysorbate 80 (PS-80) is part of drug substance. PS-80 functions as a surfactant in the drug product.
^{b} Equivalent to 0.25 mg aluminum per dose

### The Effect of Polysorbate 80 Concentration

Polysorbate 80 (PS-80) is a non-ionic surfactant. It is used to stabilize and solubilize MnB rLP2086 subfamily A and B proteins in the formulation by preventing aggregation and adsorption that may be caused by temperature, filter, tubing, container/closure contact and process mixing. Stability studies using an in vitro monoclonal antibody based potency assay demonstrated instability of the subfamily B protein at higher molar ratios of PS-80 to MnB rLP2086 protein in the final formulation. Experiments with varying molar ratios of PS-80 to protein have demonstrated that the optimal molar ratio of PS-80 to MnB rLP2086 protein is approximately between 1.4 to 4.2 to retain potency.

### Example 3: Description and Composition of the MenACWY-TT Composition

MenACWY-TT drug product is composed of the purified polysaccharides of Neisseria meningitidis serogroups A, C, W and Y, each conjugated to Tetanus Toxoid (TT) at ratios to polysaccharide of ˜3, ˜3, ˜1.5 and ˜1.3, respectively.

The MenACWY-TT drug product is presented as a lyophilized powder, supplied in a 3 mL glass vial with bromobutyl rubber closures suitable for lyophilization and aluminum flip-off caps. All components used in the manufacture of the MenACWY-TT Drug product and their functions are provided in Table 3.

TABLE 3
Composition of MenACWY-TT Drug product

| Ingredients | Quantity/dose | |
|---|---|---|
| MenA_{AH}-TT conjugate | 5 | mcg MenA |
| (mean TT/polysaccharide ratio: ~3) | ~15 | mcg TT |
| MenC_{AH}-TT conjugate | 5 | mcg MenC |
| (mean TT/polysaccharide ratio: ~3) | ~15 | mcg TT |
| MenW-TT conjugate | 5 | mcg MenW |
| (mean TT/polysaccharide ratio: ~1.5) | ~7.5 | mcg TT |
| MenY-TT conjugate | 5 | mcg MenY |
| (mean TT/polysaccharide ratio: ~1.3) | ~6.5 | mcg TT |
| Tris-HCl | 97 | mcg |
| Sucrose | 28 | mg |
| Sodium Chloride^{a} | 306.0-325.0 | mg |

^{a}Lyophilized cake also contains sodium chloride resulting from the salt present in each of the bulk purified TT conjugates. Sodium chloride concentration varies between 10.5-11.1 mM based on the composition of the clinical and commercial lots.

### Example 4: Preparation of the MenABCWY Composition

The final MenABCWY composition is prepared in the clinic by reconstituting the lyophilized MenACWY-TT drug product vial with 0.67 mL of MnB Bivalent rLP2086. The resulting MenABCWY composition (a vaccine liquid drug product) contains rLP2086 subfamily A and B proteins at 120 mcg/mL/subfamily, purified polysaccharides of Neisseria meningitis types A, C, W and Y at concentration of 10 mcg/mL/type conjugated to Tetanus Toxoid at ratios of 3, ˜3, ˜1.5, and ˜3 respectively in 10 mM histidine and 1.6 mM tris buffer containing 160.5-161.1 mM sodium chloride, 0.5 mg/mL aluminum as aluminum phosphate (AlPO4), 0.035 mg/mL polysorbate 80 and 56 mg/mL sucrose at pH of 6.05 for intramuscular injection.

The MenABCWY vaccine is prepared by mixing of two drug products, MenACWY-TT and MnB Bivalent rLP2086. Buffering components and excipients were chosen based on the individual development of each component and are shown to provide the necessary stability profile for extended shelf life.

Dosage verification studies were performed to demonstrate that MenACWY-TT drug product and MnB Bivalent rLP2086 drug product are compatible when mixed together for administration of MenABCWY vaccine and that all drug product and dosing solutions are compatible with the administration components and that dosing solutions are stable in the administration components for a period of time adequate to perform the dose preparation and administration operations. The stability of MenABCWY vaccine prepared by reconstitution of MenACWY-TT drug product with 0.67 mL of MnB Bivalent rLP2086 drug product over the hold time at ambient temperature and light conditions was confirmed in reconstituted vials and in dosing syringes.

Samples representing the dosing solutions of MenABCWY vaccine were tested using stability indicating methods such as RP-HPLC for antigen binding and purity, bioplex activity assay, ELISA, and ICP-MS with predefined acceptance criteria. The results of this study show acceptable stability of MenABCWY vaccine for 24 hours at room temperature and light conditions.

### Example 5: Evaluation of the MenABCWY Vaccine

A study was carried out to assess whether there is acceptable physical compatibility and short-term stability when a lyophilized MenACWY-TT composition is reconstituted with the MnB bivalent rLP2086 composition. The lyophilized MenACWY-TT composition and the liquid MnB bivalent rLP2086 composition were combined and stored for up to 24 hours in an uncontrolled room temperature environment to approximate real life conditions. It was demonstrated that lyophilized MenACWY-TT composition could be reconstituted with the liquid MnB bivalent rLP2086 composition with gentle hand mixing and the combined pH and osmolality were within typical range for an injectable. All key attributes for the conjugates and proteins were similar to those of a control for up to 24 hours in the uncontrolled room temperature environment.

The physical compatibility was evaluated through assessing pH, appearance, ease of reconstitution, and osmolality of the combined drug product. The stability of the antigens was evaluated through assessing concentration, purity and the in-vitro relative antigenicity (IVRA) of the rLP2086 subfamily A and subfamily B proteins as well as the concentrations of the conjugated Meningococcal A, C, Y, and W-135 polysaccharides by ELISA.

Example 5 through Example 15 demonstrate that the combination of the lyophilized MenACWY-TT composition and the liquid MnB bivalent rLP2086 composition, i.e., the MenABCWY composition, was found to be compatible and stable for at least 24 hours at room temperature.

### ELISAs for Determining Mening a, C, Y, and W-135 Polysaccharide Concentrations in the MenABCWY Composition—Development of the Mening a, C, Y and W-135 ELISA & Screening of the pAb for Detection

Six antibodies were selected for screening for use in the ELISA assays. Each of four groups of ten rabbits was immunized with either Men A, C, Y or W-135 polysaccharide TT conjugates with rabbits subsequently exsanguinated after antibody development. Each rabbit was individually screened using Men A, C, Y or W-135 polysaccharides conjugated to a carrier protein CRM₁₉₇ for binding and specificity. Rabbit sera was screened for a positive binding signal, which is equivalent to an absorbance reading greater than three-fold above the background absorbance. Additionally, rabbit sera was screened for low non-specific binding, which was any absorbance readings for sera combinations without antigen, secondary or detection above the absorbance reading for the blank, as well as low cross-reactivity, which was any absorbance readings for heterologous serotypes that was above the background absorbance. Rabbits that met the screening criteria were pooled. The standard curve range was established using CRM conjugates and confirmed with reconstituted the lyophilized MenACWY-TT composition. The standard curve range was established using CRM conjugates and confirmed with reconstituted lyophilized MenACWY-TT composition.

The feasibility of quantitating the A, C, Y and W conjugates in the combined drug product (MenABCWY composition) was established. It was determined that the MnB Bivalent rLP2086 composition alone was not detected in the assay. Additionally, when aluminum phosphate in the MenABCWY composition samples was solubilized, full recovery of conjugates was obtained. Therefore, it was determined that the MnB bivalent rLP2086 composition does not interfere with the quantitation of the MenACWY-TT conjugates by ELISA.

### Example 6: Evaluation of Suitability of Methods to Assess the MnB Bivalent rLP2086 Composition in the Presence of the MenACWY-TT Composition

IEX-HPLC was evaluated for its suitability to determine the strength of the MnB bivalent rLP2086 composition subfamily A and B proteins in the presence of the MenACWY-TT composition. The total protein and bound protein results for the MnB bivalent rLP2086 composition in the presence of the MenACWY-TT composition and in the absence of the MenACWY-TT composition were accessed.

TABLE 4

| Sample | Subfamily | Bound Protein, % |
|---|---|---|
| the MnB bivalent | Protein A | 107 |
| rLP2086 composition | (SEQ ID NO: 1) | |
| no the MenACWY- | Protein B | 104 |
| TT composition | (SEQ ID NO: 2) | |
| the MnB bivalent | Protein A | 108 |
| rLP2086 composition | (SEQ ID NO: 1) | |
| with the MenACWY- | Protein B | 103 |
| TT composition | (SEQ ID NO: 2) | |

### Example 7: Evaluation of the MnB Bivalent rLP2086 Composition Purity and Peak Ratio in the Presence of the MenACWY-TT Composition

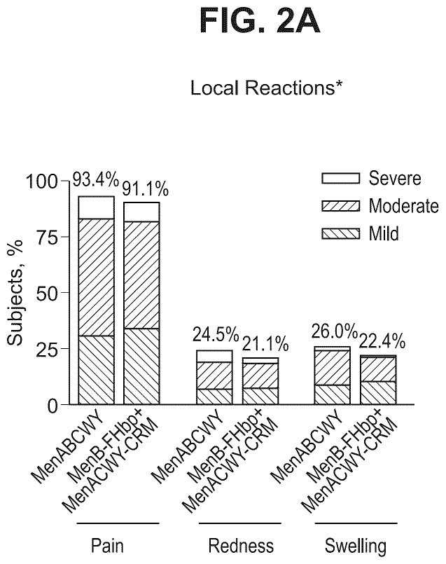

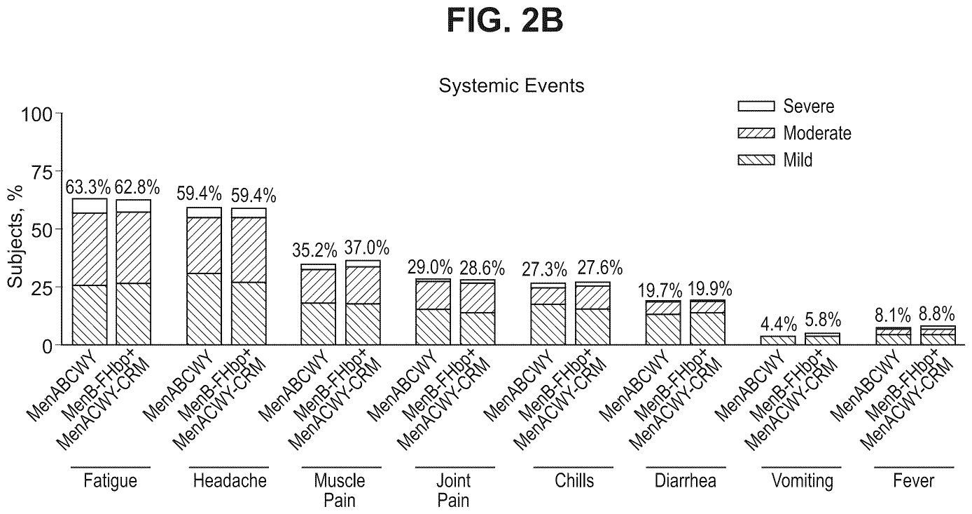

RP-HPLC was evaluated for its suitability to determine the purity of the MnB bivalent rLP2086 composition in the presence of the MenACWY-TT composition. The purity results for the MnB bivalent rLP2086 composition in the presence of the MenACWY-TT composition and in the absence of the MenACWY-TT composition were compared. The overlaid chromatograms are shown in FIG. 2 of U.S. Pat. No. 10,183,070. An example of the integration of the impurity peak is shown as an insert in FIG. 2 of U.S. Pat. No. 10,183,070. The evaluation results show that the presence of the MenACWY-TT composition does not interfere with evaluation of the MnB bivalent rLP2086 composition purity using the RP-HPLC method.

### Example 8: Evaluation of the MnB Bivalent rLP2086 Composition IVRA in the Presence of the MenACWY-TT Composition

The IVRA method was evaluated for its suitability for determination of in-vitro relative antigenicity of the MnB bivalent rLP2086 composition Subfamily A (SEQ ID NO: 1) and Subfamily B (SEQ ID NO: 2) proteins in the presence of the MenACWY-TT composition.

The IVRA results for the MnB bivalent rLP2086 composition Subfamily A and Subfamily B proteins in the presence and in the absence of the MenACWY-TT composition were compared. The feasibility evaluation results show that, within the assay variability the results are comparable and that the presence of the MenACWY-TT composition does not interfere with determination of in-vitro relative antigenicity.

### Example 9: Reconstitution of the MenACWY-TT Composition Vials with the MnB Bivalent rLP2086 Composition

The MenACWY-TT composition and the MnB bivalent rLP2086 composition drug products were performed using the MenACWY-TT composition vials reconstituted with the MnB bivalent rLP2086 composition drug product. The MenACWY-TT composition vials reconstituted with either saline or the MenACWY-TT composition matrix placebo were used as controls depending on the method.

TABLE 5

| Product | Component | Composition | |
|---|---|---|---|
| The MenACWY- | Neisseria meningitidis | | |
| TT composition | Group A polysaccharide | 10 | μg/mL |
| | Group C polysaccharide | 10 | μg/mL |
| | Group W-135 polysaccharide | 10 | μg/mL |
| | Group Y polysaccharide | 10 | μg/mL |
| | Tetanus toxoid carrier protein | 88 | μg/mL |
| | Sucrose | 164 | mM |
| | Trometanol | 1.6 | mM |
| the MnB bivalent | Sub-family A rLP2086 protein | 120 | μg/mL |
| rLP2086 | (SEQ ID NO: 1) | | |
| composition | Sub-family B rLP2086 protein | 120 | μg/mL |
| | (SEQ ID NO: 2) | | |
| | AlPO4 | 0.5 | mg/mL |
| | Histidine | 10 | mM |
| | NaCl | 150 | mM |
| | | pH 6.0 | |
| the MnB bivalent | AlPO4 | 0.5 | mg/mL |
| rLP2086 | Histidine | 10 | mM |
| composition | NaCl | 150 | mM |
| Placebo | | pH 6.01 | |
| Bulk MnB | Sub-family A rLP2086 protein | 120 | μg/mL |
| bivalent rLP2086 | (SEQ ID NO: 1) | | |
| composition DP | Sub-family B rLP2086 protein | 120 | μg/mL |
| | (SEQ ID NO: 2) | | |
| | AlPO4 | 0.5 | mg/mL |
| | Histidine | 10 | mM |
| | NaCl | 150 | mM |

pH 6.0

### Determination of Saline Reconstitution Volume for the MenACWY-TT Composition

The NIMENRIX® commercial product package contains both a vial containing the lyophilized MenACWY-TT composition and a syringe containing 0.9% saline used for reconstitution. In order to reproduce the final NIMENRIX® concentration in the commercial vaccine upon reconstitution with the MnB bivalent rLP2086 composition, the amount of saline dispensed using the syringe from the commercial product had to be determined. This same volume of the MnB bivalent rLP2086 composition would then be used for all reconstitution studies.

Reconstitution of the MenACWY-TT Composition Vials with the MnB Bivalent rLP2086 Composition

The MnB bivalent rLP2086 composition was pooled in a 10 mL glass vial. Approximately 800 μL of the solution was withdrawn into a 1 mL syringe. The adjusted contents of the syringe were injected into a vial containing the MenACWY-TT composition. The vial was swirled to dissolve the contents.

The pH and appearance were determined on duplicate samples on the MenABCWY composition. Osmolality was measured in triplicate on the MenACWY-TT composition reconstituted with saline and on the MenACWY-TT composition reconstituted with the MnB bivalent rLP2086 composition.

### Example 10: SEC-MALLS to Evaluate Mening a, C, Y, and W-135 Polysaccharide Stability in DP Matrix

Mening A, C, Y, and W-135 Polysaccharides were used as surrogates to assess if any instability of the conjugated Meningococcal A, C, Y, and W-135 polysaccharides in the combined drug product (the MenABCWY composition) could be expected.

### Treatment of Mening a, C, Y, and W-135 Polysaccharides

### Reagent Preparation (“Full MenABCWY Composition Buffer Matrix”)

2.24 g of sucrose and 7.8 mg of Tris (Tromethamine) was added to 20 ml of 2×MnB bivalent rLP2086 composition buffer matrix with MnB rLP2086 proteins (Histidine 20 mM pH 6.0, NaCl 300 mM, PS 80 0.07 mg/ml, AlPO4 1 mg/ml (8 mM), rLP2086 subfamily A (SEQ ID NO: 1) and subfamily B (SEQ ID NO: 2) proteins 240 μg/mL each).

### Sample Preparation

Each Mening Polysaccharide was diluted 1:1 with Full MenABCWY composition Buffer Matrix and incubated for 0, 6 and 24 hours at 5° C., 25° C. and 37° C. After incubation the sample suspension was spun for 1 minute at 14,000 r.p.m. The supernatant was analyzed by SEC-MALLS.

### Example 11: Stability of the MenABCWY Composition—Evaluation of pH, Appearance, and Osmolality of the Combined MnB Bivalent rLP2H86 and MenACWY-TT Compositions

The pH and appearance of the combined MnB bivalent rLP2086 composition and the MenACWY-TT composition, i.e., the MenABCWY composition, were evaluated immediately after reconstitution and again after 24 hours.

All results were as expected (Table 6).

TABLE 6
Appearance and pH of the MenABCWY composition

| | | Time Point, | | |
|---|---|---|---|---|
| Sample # | Sample | hours | Appearance | pH |
| 1 | MenABCWY | 0 | Homogeneous | 5.8 |
| | composition, | | white | |
| | Rep1 | | suspension | |
| 2 | MenABCWY | 0 | Homogeneous | 5.8 |
| | composition, | | white | |
| | Rep2 | | suspension | |
| 3 | MenABCWY | 24 | Homogeneous | 5.8 |
| | composition, | | white | |
| | Rep 1 | | suspension | |
| 4 | MenABCWY | 24 | Homogeneous | 5.8 |
| | composition, | | white | |
| | Rep 2 | | suspension | |
| 5 | MenACWY-TT | 0 | Clear, | 6.3 |
| | composition | | Colorless | |
| | w/Saline | | | |
| 6 | MenACWY-TT | 24 | Clear, | 6.4 |
| | composition | | Colorless | |
| | w/Saline | | | |

The average osmolality of the MenACWY-TT composition reconstituted with the MnB bivalent rLP2086 composition was within 3% of the average osmolality of the MenACWY-TT composition reconstituted with saline.

TABLE 7

| | Recon- | | | | |
|---|---|---|---|---|---|
| | stituting | Reading | Reading | Reading | Average |
| Vial | Agent | 1 mOsm | 2 mOsm | 3 mOsm | mOsm |
| MenACWY- | Saline | 471 | 473 | 478 | 474 |
| TT | | | | | |
| composition | | | | | |
| MenACWY- | MnB | 487 | 487 | 489 | 488 |
| TT | bivalent | | | | |
| composition | rLP2086 | | | | |
| | composition | | | | |

### Example 12: Mening a, C, Y, and W-135 Polysaccharide Conjugate Concentrations in the Combined Drug Product

The concentration of the Mening A, C, Y and W-135-TT conjugates in the MenABCWY composition was assessed initially and again after 24 hours. The concentrations of the four conjugates were stable over the twenty four hour time period (Table 8).

TABLE 8
Short Term Stability Results of the MnA, C, Y and W Conjugates
in the MenABCWY composition by ELISA

| | MenACWY-TT composition + | | | The MenACWY-TT | | |
|---|---|---|---|---|---|---|
| | MnB bivalent rLP2086 | | | composition + | | |
| | composition | | | Saline | | |
| | | After 24 | | | After 24 | |
| | Initial, | hrs, | Stability | Initial, | hrs, | Stability |
| Serotype | μg/mL | μg/mL | Ratio | μg/mL | μg/mL | Ratio |
| A | 6.7 | 6.8 | 1.0 | 6.4 | 6.7 | 1.1 |
| C | 6.9 | 6.6 | 1.0 | 6.5 | 6.7 | 1.0 |
| Y | 8.1 | 8.7 | 1.1 | 9.6 | 9.8 | 1.0 |
| W | 8.5 | 8.8 | 1.0 | 8.8 | 9.0 | 1.0 |

### Example 13: Evaluation of the Stability of the MnB Bivalent rLP2086 Proteins in the MenABCWY Composition

Total and Bound rLP2086 Subfamily A (SEQ ID NO: 1) and Subfamily B (SEQ ID NO: 2) Protein Concentrations in the Combined Drug Product

The MenABCWY composition samples were analyzed by IEX-HPLC to determine the protein concentrations. As shown in Table 9, the total protein, bound protein (to aluminum), and % bound of both MnB bivalent rLP2086 Subfamily A (SEQ ID NO: 1) and Subfamily B (SEQ ID NO: 2) proteins (bound to aluminum) did not change within 24 hours indicating that the rLP2086 Subfamily A and Subfamily B proteins were stable over the twenty four hour time period.

TABLE 9
Total and Bound Protein Stability

| Time | | Total | Bound | Bound |
|---|---|---|---|---|
| Point, | | Protein, | Protein, | Protein, |
| hours | Subfamily | μg/mL | μg/mL | % |
| 0 | A | 83 | 85 | 102 |
| | B | 88 | 87 | 99 |
| 24 | A | 87 | 88 | 101 |
| | B | 92 | 91 | 99 |

### Example 14: RLP2086 Protein Purity and Peak Ratio in the Combined MenABCWY Composition

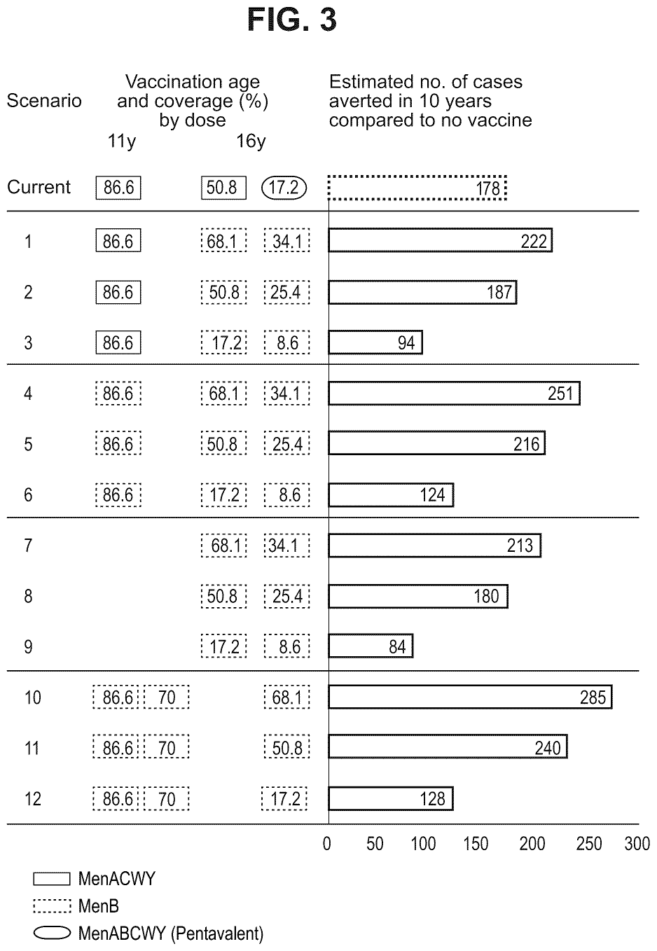

The MenABCWY composition samples were analyzed by RP-HPLC to determine purity and peak ratios for the rLP2086 proteins. See FIG. 3 of U.S. Pat. No. 10,183,070. The peak at 11.9 min is excluded from the purity calculation.

rLP2086 Subfamily a and Subfamily B Protein IVRA in the Combined Drug Product

The IVRA of the MenABCWY composition samples was assessed for up to 24 hours after mixing. It was determined that the relative antigenicity of the rLP2086 Subfamily A (SEQ ID NO: 1) and Subfamily B (SEQ ID NO: 2) proteins in the MenABCWY composition was stable over the twenty four hour time period.

### Example 15: Mening a, C, Y, and W-135 Polysaccharide Stability in Full MenABCWY Composition Buffer Matrix by SEC-MALS

Stability of Mening a PS in Full MenABCWY Composition Buffer Matrix by SEC-MALLS after 6 and 24 Hours Incubation at Various Temperatures

Mening A, C, W, and Y polysaccharides were mixed with the Full MenABCWY Composition Buffer Matrix and evaluated for stability by SEC-MALS after incubation at 5° C., 25° C. and 37° C. C for up to 24 hours. All four polysaccharides appear to be stable for up to 24 hours at 5° C. and 25° C. Some degradation was observed at 37° C. for Mening A and Y. The degree of degradation could not be determined for Mening Y Polysaccharides due to formation of high Mw aggregates under all tested conditions except initial.

TABLE 10

| | Incubation | Incubation | | | |
|---|---|---|---|---|---|
| Sample | Time, hours | Temperature | | Mw (kDa) | Δ Mw (%) |
| Mening A PS | 0 | NA | | 169 | N/A |
| | 24 | 5° | C. | 171 | 1 |
| | | 25° | C. | 157 | −7 |
| | | 37° | C. | 126 | −26 |
| Mening C PS | 0 | NA | | 213 | N/A |
| | 24 | 5° | C. | 216 | 1 |
| | | 25° | C. | 215 | 1 |
| | | 37° | C. | 220 | 3 |
| Mening Y PS* | 0 | NA | | 294 | N/A |
| | 24 | 5° | C. | 734 | 149 |
| | | 25° | C. | 756 | 157 |
| | | 37° | C. | 696 | 136 |
| Mening W-135 PS | 0 | NA | | 205 | N/A |
| | 24 | 5° | C. | 230 | 12 |
| | | 25° | C. | 211 | 3 |
| | | 37° | C. | 219 | 7 |

### Example 16: Assessment of the Neisseria meningitidis Serogroup B Immunogenicity of Mn Pentavalent and Trumenba® Vaccines in CBA/J Mice

The immune response to Neisseria meningitidis serogroup B fHBP following vaccination with either bivalent Mn B fHBP vaccine, Trumenba, or the bivalent Mn B fHBP vaccine formulated with quadrivalent ACWY polysaccharide conjugate vaccine (Mn Pentavalent ABCWY) was evaluated in CBA/J mice. Groups of CBA/J mice were immunized with 3 different vaccines: Pentavalent (ABCYW), Trumenba® (MnB) and Nimenrix® (ACYW) (Table 11).

TABLE 11
Study Design: Dose Levels for each Vaccine

| | Dose Levels, μg/0.25 mL Dose | | | |
|---|---|---|---|---|
| Dilution | Mn Pentavalent | TRUMENBA ® | NIMENRIX ® | AlPO₄ |
| Factor | (ABCWY) | (B) | (ACWY) | (diluent) |
| 1 | 8 + 1.33 | 8 | 1.33 | 125 |
| 2 | 4 + 0.67 | 4 | 0.67 | 125 |
| 4 | 2 + 0.33 | 2 | 0.33 | 125 |
| 8 | 1 + 0.17 | 1 | 0.17 | 125 |

For each arm, CBA/J mice (25/group) were subcutaneously immunized in the scruff of the neck using 2-fold dilution dose levels of the respective vaccine (Table 11). Mice were primed with the vaccine at time 0 and boosted at week 2. Sera were collected PD2 at week 3 for testing using two different serum bactericidal assays that utilized human complement (hSBA). One hSBA used an fHBP subfamily A expressing strain (M98250771) and the other an fHBP subfamily B expressing strain (CDC1127).

The hSBA measures antibody-dependent, complement mediated bactericidal activity against N meningitidis serogroup B strains. Briefly, test sera at the appropriate dilution were mixed in 96-well microtiter assay plates with freshly prepared bacterial cultures of the N meningitidis B strains (subfamily A or B) and human complement. Assay plates were placed on an orbital shaker and mixed for 30 min in a humidified incubator (37° C./5% CO₂). Subsequently, aliquots of the assay reaction from each well were transferred to 96-well filter plates for enumeration of surviving bacteria.

Response rates to vaccination were calculated as the percentage of mice in each dosing group (n=25) that respond in hSBAs. When tested at a predetermined dilution level, mouse serum samples that kill ≥50% of the T₃₀ control meningococcal bacteria are considered responders. The T₃₀ control wells contain bacteria and complement but no test serum and are counted at the end of the 30 minute assay incubation.

### Table 12 and Table 11

Table 13 show comparable dose-dependent response rates induced by either TRUMENBA® or Mn Pentavalent for both subfamily A and subfamily B of the N meningitidis serotype B strains. As expected, NIMENRIX™ did not induce a functional immune response to Mn B strains.

TABLE 12
Subfamily A hSBA responses (% responders)

| Dilution | | | |
|---|---|---|---|
| Factor ^{a} | TRUMENBA | NIMENRIX | Penta |
| 8 | 24% | 0% | 8% |
| 4 | 40% | 0% | 16% |
| 2 | 52% | 0% | 56% |
| 1 | 80% | 0% | 92% |

^{a} See corresponding dose levels in Table 11

TABLE 13
Subfamily B hSBA responses (% responders)

| Dilution | | | |
|---|---|---|---|
| Factor ^{a} | TRUMENBA | NIMENRIX | Penta |
| 8 | 28% | 0% | 36% |
| 4 | 56% | 0% | 60% |
| 2 | 60% | 0% | 76% |
| 1 | 72% | 0% | 76% |

^{a} See corresponding dose levels in Table 11

### Example 16: a Study to Describe the Immunogenicity, Safety, and Tolerability of a Bivalent rLP2086-Containing Pentavalent Vaccine (MenABCWY) in Healthy Subjects 10 to <26 Years of Age (B1971057)

B1971057 is a Phase 2 proof-of concept (POC) study to assess the safety and immunogenicity of Penta in healthy subjects ≥10 to <26 years of age. The study was initiated in April 2017 with approximately 530 subjects received Penta.

Meningococcal vaccines are licensed by an immunological surrogate, the serum bactericidal assay using human complement (hSBA) that demonstrates the ability of immune sera to kill meningococcal strains that represent the serogroups included in the vaccine. For MenACWY responses, one strain from each serogroup were evaluated in hSBAs and Penta responses were compared to the licensed ACWY vaccine MENVEO. For the MenB evaluation; 4 serogroup B strains were tested that were also used during the TRUMENBA licensure studies.

The study data showed Penta to be non-inferior to Menveo after 1 vaccination for the ACWY evaluation and for Penta to be non-inferior to Trumenba after 2 vaccinations for the B evaluation.

### Investigational Penta Vaccine

For this study, the investigational products are bivalent rLP2086 (TRUMENBA), (Penta; described in Example 1 and Example 4 above), MenACWY-CRM (MENVEO), and placebo.

### Study Design

B1971057 is a Phase 3, randomized, active-controlled, observer-blinded multicenter trial in which approximately 1590 subjects were randomly assigned to receive either Penta and placebo (saline), or Trumenba (Pfizer), and Menveo (GSK). All subjects were naive to any meningococcal group B vaccine prior to enrollment. Randomization was stratified by prior vaccination history; ˜50% ACWY-naive subjects and ˜50%. ACWY-experienced (having received 1 prior dose of a vaccine containing 1 or more ACWY Groups ≥4 years prior to the date of randomization). This ACWY experienced group was included because in the US 86% of teenagers receive a dose of an ACWY vaccine at approximately 11 years of age and should receive a booster dose at 16 years of age. Randomization was also stratified by geographic region. Approximately 80% subjects from US investigative sites and approximately 20% subjects from Europe. Regional stratification ensured sufficient population representation. The study was conducted in 2 stages. Stage 1, now completed, comprised the vaccination phase of a primary series. The visit schedule for Stage 1 is noted in Table 14 below. Stage 2 will evaluate persistence of immunity and a booster dose administered approximately 4 years after completion of the primary series of Penta.

TABLE 14
B1971057 Stage 1 Study Design

| | | | Post | | Post | | Stage 2 |
|---|---|---|---|---|---|---|---|
| | | | Vaccination | | Vaccination | Safety | Progression |
| | | Vaccination | 1 Blood | Vaccination | 2 Blood | Telephone | Telephone |
| | | 1 | Draw | 2 | Draw | Call | call |
| | | Month | | | | | |
| | | 0 | 1 | 6 | 7 | 12 | 12-18 |
| | | Visit | | | | | |
| | | 1 | 2 | 3 | 4 | 5 | 6 |
| ACWY | Group | Penta + | | Penta | | | |
| Naïve | 1 | Saline | | | | | |
| Subjects | (n~265) | | | | | | |
| | Group | Trumenba + | | Trumenba | | | |
| | 2 | Menveo | | | | | |
| | (n~530) | | | | | | |
| ACWY | Group | Penta + | | Penta | | | |
| Naïve | 3 | Saline | | | | | |
| Subjects | (n~265) | | | | | | |
| | Group | Trumenba + | | Trumenba | | | |
| | 4 | Menveo | | | | | |
| | (n~530) | | | | | | |

For assessment of the immune response, functional antibodies were analysed in hSBAs with meningococcal group A, B, C, W, and Y strains. The hSBA measures antibodies in human sera that result in complement-dependent killing of the target meningococcal strain. For assessment of the immune response to Trumenba and the B component of Penta, 4 primary MnB test strains, PMB80 (A22), PMB2001 (A56), PMB2948 (B24), and PMB2707 (B44), were used in the hSBAs for determination of the immunogenicity endpoints in this study. For assessment of the immune response to MENVEO and the ACWY components of Penta, test strains specific for each of the ACWY groups were identified and qualified in hSBAs prior to the commencement of testing. The validated assays used to evaluate MenB were the same as the ones used to license TRUMENBA.

### Objectives

This study was designed to describe the safety, tolerability, and immunogenicity of Penta, and describe the immune response to groups A, B, C, W, and Y following administration of Penta, or Trumenba and Menveo a US licensed meningococcal group A, C, W-135, and Y conjugate vaccine. Nimenrix was not used as the ACWY comparator; it is not licensed in the US.

The immunogenicity of the MenACWY component of Penta was based on the hSBA geometric mean titers (GMTs) of Penta after a single dose when compared to hSBA GMTs observed for Trumenba+Menveo after 1 dose.

We conducted this assessment for individuals who had not previously received a dose of a MenACWY vaccine (ACWY naive individuals) in addition to individuals who would be receiving their second MenACYW booster dose (ACWY experienced individuals). The POC criteria for the MenACWY component of Penta needed to be met for ACWY naive individuals.

The criteria for immunogenicity of the MenB component of Penta was based on achieving point estimates of the 4-fold rise and composite hSBA responses after 2 doses of Penta administered on a 0-, 6-month schedule that predict meeting the Phase 3 LCI criteria established for the 0,6-month schedule.

Secondary endpoints for the study included the standard noninferiority evaluations of the ratio of 2 GMTs being with a 2.0-fold margin and percent responders for B component of Penta. The percent responder analysis was conducted by defining a responder as achieving a 4-Fold Rise in hSBA Titer and Composite Response 1 Month After Vaccination 2 for Primary MenB Strains and then calculating the difference between Penta and Trumenba. A difference no larger than 10% was required to achieve success.

### Immune Response to Penta and POC Immunogenicity Results

Study B1971057 shows in the following, with respect to the bactericidal responses against MenACWY between Penta and Menveo: similar hSBA GMTs for the ACWY components were observed for subjects who received Penta compared to Menveo after a single dose. A 1.5-fold GMT ratio non-inferiority (NI) margin was achieved in ACWY naive subjects (Table 15) and a 2.0-fold GMT ratio NI margin was achieved in ACWY experienced (Table 16) subjects.

TABLE 15
Penta ACWY hSBA GMTs compared to Menveo were within the 1.5-fold
GMR NI margin after a single dose in ACWY Naïve Subjects

| | | | | Group 2 | | | Ratio | |
|---|---|---|---|---|---|---|---|---|
| | | | | (Trumenba + | | | (Gr 1 vs | |
| Strain | Group 1 (Penta) | | | Menveo | | | Gr 2) | |
| (Variant) | | | (95% | | | (95% | | (95% |
| Timepoint | N | GMT | CI) | N | GMT | CI) | GMR | CI) |
| Men A | | | | | | | | |
| 1 Month after | 264 | 215.8 | (184.6, | 510 | 203.2 | (178.7, | 1.06 | (0.86, |
| Vaccination 1 | | | 252.4) | | | 231.0) | | 1.31) |
| MenC | | | | | | | | |
| 1 Month after | 262 | 111.5 | (87.2, | 509 | 81.4 | (68.1, 97.4) | 1.37 | (1-01, |
| Vaccination 1 | | | 142.6) | | | | | 1.86) |
| MenW | | | | | | | | |
| 1 Month after | 264 | 98.4 | (80.7, | 512 | 71.2 | (61.5, 82.4) | 1.38 | (1.08, |
| Vaccination 1 | | | 120.0) | | | | | 1.77) |
| MenY | | | | | | | | |
| 1 Month after | 263 | 141.9 | (118.8, | 510 | 96.6 | (83.9, | 1.47 | (1.16, |
| Vaccination 1 | | | 169.4) | | | 111.2) | | 1.85) |

POC GMR point estimates for Penta vs Menveo ≥0.612 for MenA;
≥0.67 for MenC;
≥0.635 for MEnW and
≥0.626 for MenY.
All achieved.
Penta Non-inferiority for Menveo is established in ACWY naïve subjects at the 1.5-fold GMR margin as GMR LCIs for all serogroups are > 0.67
Penta statistically greater to Menveo for Serogroups C, W and Y

TABLE 16
Penta ACWY hSBA GMTs compared to Menveo were within the 1.5-fold
GMR NI margin after a single dose in ACWY Experienced Subjects

| | | | | Group 4 | | | Difference | |
|---|---|---|---|---|---|---|---|---|
| | Group 3 | | | (Trumenba + | | | (Gr 3 vs | |
| Strain | (Penta) | | | Menveo | | | Gr 4) | |
| (Variant) | | | (95% | | | (95% | | (95% |
| Timepoint | N | GMT | CI) | N | GMT | CI) | GMR | CI) |
| MenA | | | | | | | | |
| 1 Month after | 218 | 568.6 | (492.9, | 411 | 916.1 | (809.1, | 0.62 | (0.51, |
| Vaccination 1 | | | 656.0) | | | 1037.3) | | 0.76) |
| MenC | | | | | | | | |
| 1 Month after | 264 | 814.9 | (689.4, | 506 | 827.0 | (722.5, | 0.99 | (0.79, |
| Vaccination 1 | | | 963.2) | | | 946.6) | | 1.23) |
| MenW | | | | | | | | |
| 1 Month after | 219 | 1214.9 | (1032.0, | 414 | 1176.7 | (1017.9, | 1.03 | (0.82, |
| Vaccination 1 | | | 1430.1) | | | 1360.2) | | 1.30) |
| MenY | | | | | | | | |
| 1 Month after | 218 | 1174.0 | (990.3, | 413 | 1000.2 | (872.1, | 1.17 | (0.94, |
| Vaccination 1 | | | 1391.9) | | | 1147.1) | | 1.47) |

Penta Non-inferiority to Menveo is established in this study at the 2.0 fold GMR margin as GMR LCIs for all serogroups are >0.5 in subjects with ACWY background

Trumenba was licensed based on the proportion of subjects achieving 4-fold rise (and composite response) in antibody titers that met a prespecified Lower Limit 95% Confidence Interval (LCI) threshold. When 4-fold antibody responses are assessed they can be influenced by background titers in the population which may independently influence the proportion of subjects that have a 4-fold response to the vaccine. In addition, the hSBA assays used are comprised of biological components that even though tightly controlled, may affect absolute response criteria from study to study. Taking into consideration that the licensure LCI thresholds were calculated based on point estimates achieved in a different population and using different complement sources, we also compared Penta serogroup B responses to those from the Trumenba+Menveo arms. The results are provided in Table 17.

TABLE 17
4-Fold Rise and Composite Response All Ages
(10 to <26) 1 Month Post Dose 2.

| | Group 1 + | | | Group 2 + | | | POC Criteria |
|---|---|---|---|---|---|---|---|
| | 3 Combined | | | 4 Combined | | | Point |
| Endpoint Strain | (Penta) | | | (Trumenba) | | | Estimate |
| (Variant) | N | % (n) | (95% CI) | N | % (n) | (95% CI) | 0, 6 Month |
| 4-fold Rise | | | | | | | |
| from Baseline | | | | | | | |
| PMB80 (A22) | 422 | 75.8 (320) | (71.5, | 827 | 73.8 (610) | (70.6, | 78.1 |
| | | | 79.8) | | | 76.7 | |
| PMB2001 (A56) | 418 | 94.7 (396) | (92.1, | 823 | 95.0 (782) | (93.3, | 87.5 |
| | | | 96.7) | | | 96.4) | |
| PMB2948 (B24) | 430 | 69.3 (298) | (64.7, | — | — | — | 58.6 |
| | | | 73.6) | | | | |
| PMB2707 (B44) | 432 | 91.7 (396) | (88.6, | 850 | 86.4 (734) | (83.9, | 63.6 |
| | | | 94.1) | | | 88.6) | |
| Composite | 424 | 76.7 (325) | (72.3, | — | — | — | 68.5 |
| response | | | 80.6) | | | | |
| (hSBA titer ≥ LLOQ | | | | | | | |
| for all 4 strains) | | | | | | | |

B24 data pending

For three of the 4 serogroup B test strains SEA 4-fold rise and composite responses considerably exceeded the prespecified point estimates. For one strain, PMB80(A22), 75.8% of subjects achieved a 4-fold rise in titers (for the Trumenba group the point estimate (PE) was 73.8), compared to the POC criteria PE of 78.1%; however, as stated above, the 95% LCI criteria are more difficult to control in different populations and over time. The phase 2 study used to create LCI for this POC study enrolled a European population where the baseline hSBA rate for A22 was 22.1%. Since the PE of Penta was higher than that of Trumenba, it suggested that there was no immune interference of the MenB component of Penta. To confirm this point, the secondary endpoint analysis showed that Trumenba+Menveo was noninferior to Penta in 2 noninferiority analyses. Indeed, they met the stringent 1.5 GMR non-inferiority margin (Table 18) and the percent responder analysis at the 5% margin (Table 19).

TABLE 18
GMTs and GMT ratio of Penta Compared to
Trumenba 1 Month Post Dose 2.

| | | | | | | | Ratio | |
|---|---|---|---|---|---|---|---|---|
| | Groups 1 + 3 | | | Groups 2 + 4 | | | (Gr 1 + 3 vs | |
| | Combined (Penta) | | | Combined (Trumenba) | | | Gr 2 + 4) | |
| Strain | | | (95% | | | (95% | | (95% |
| (Variant) | N | % (n) | CI) | N | % (n) | CI) | % | CI) |
| PMB80 | 433 | 51.0 | (46.7, 55.7) | 852 | 49.3 | (46.2, 52.6) | 1.03 | (0.93, 1.16) |
| (A22) | | | | | | | | |
| PMB2001 | 435 | 152.3 | (138.5, 167.5) | 854 | 139.5 | (130.6, 149.1) | 1.09 | (0.97, 1.22) |
| (A56) | | | | | | | | |
| PMB2948 | 431 | 20.2 | (18.3, 22.3) | — | — | — | — | — |
| (B24) | | | | | | | | |
| PMB2707 | 436 | 43.3 | (39.1, 47.9) | 853 | 37.8 | (35.1, 40.8) | 1.14 | (1.01, 1.30) |
| (B44) | | | | | | | | |

Penta Non-inferiority to Trumenba is established in this study at the 1.5-fold GMR margin for A22, A56 and B44 as GMR LCIs are >0.67. No interference

TABLE 19
Difference in % Responders Who Achieved 4-fold response

| | Group 1 + 3 | | | Group 2 + 4 | | | Difference | |
|---|---|---|---|---|---|---|---|---|
| | Combined | | | Combined | | | (Gr 1 + 3 − | |
| Endpoint Strain | (Penta) | | | (Trumenba) | | | Gr 2 + 4) | |
| (Variant) | N | % (n) | (95% CI) | N | % (n) | (95% CI) | % | (95% CI) |
| 4-fold Rise from | | | | | | | | |
| Baseline | | | | | | | | |
| PMB80 (A22) | 422 | 75.8 (320) | (71.5, | 827 | 73.8 (610) | (70.6, | 2.1 | (−3.1, |
| | | | 79.8) | | | 76.7) | | 7.0) |
| PMB2001 (A56) | 418 | 94.7 (396) | (92.1, | 823 | 95.0 (782) | (93.3, | −0.3 | (−3.2, |
| | | | 96.7) | | | 96.4) | | 2.2) |
| PMB2948 (B24) | 430 | 69.3 (298) | (64.7, | — | — | — | — | — |
| | | | 73.6) | | | | | |
| PMB2707 (B44) | 432 | 91.7 (396) | (88.6, | 850 | 86.4 (734) | (83.9, | 5.3 | (1.7, |
| | | | 94.1) | | | 88.6) | | 8.7) |
| Composite | 424 | 76.7 (325) | (72.3, | | | | | |
| response | | | 80.6) | | | | | |
| (hSBA titer ≥ LLOQ | | | | | | | | |
| for all 4 strains) | | | | | | | | |

Penta Non-inferiority to Trumenba is established in this study at the 5% margin for A22, A56 and B44
No interference between Trumenba and Penta

### Example 17: Potential Public Health Impact in the US of a Pentavalent Vaccine Targeting Neisseria meningitidis Serogroups a, B, C, W and Y

Objective: To evaluate the potential for additional reductions of IMD cases among the US population under various assumptions of vaccination schedules and compliance rates with Pentavalent vaccine.

### Vaccination Scenarios

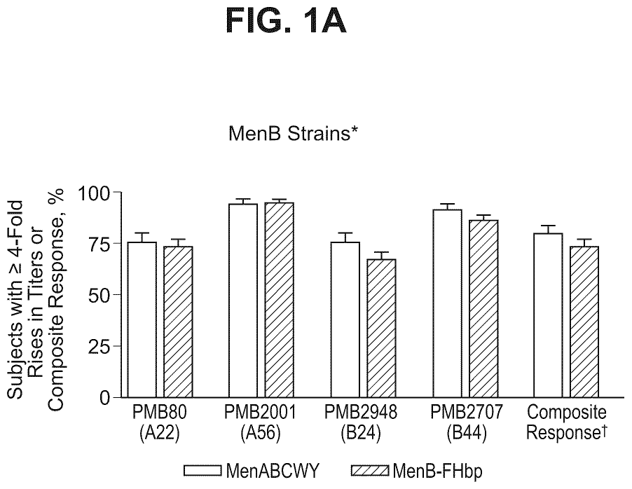

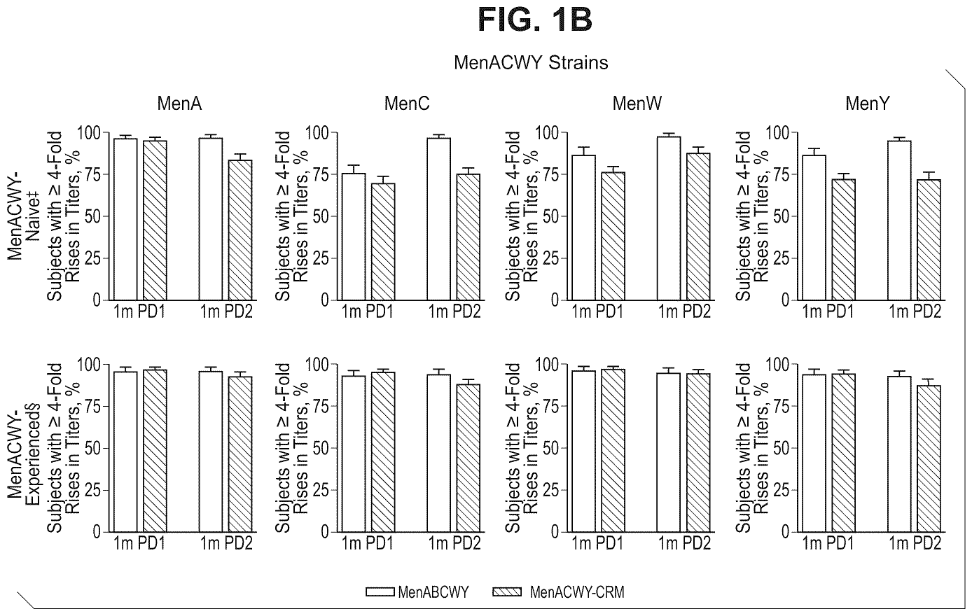

Four primary vaccination scenarios were analyzed and compared to estimated cases averted with current recommendations and compliance levels (FIG. 1)
1. Replacing MenACWY/MenB vaccines at age 16 years with Penta, and retaining MenACWY at age 11 years
2. Replacing MenACWY/MenB vaccines at age 11 years and 16 years with Penta
3. Replacing MenACWY/MenB at age 16 years with Penta, and no MenACWY vaccination at age 11 years
4. Replacing MenACWY at age 11 years with 2 doses of Penta and MenACWY/MenB at age 16 years with 1 dose of Penta

Vaccine coverage with each of the schedules was varied to estimate the impact of different recommendations on the overall levels of IMD reduction

Disease reduction estimates were based on published 2018 estimates of adolescent immunization coverage in the United States
86.6% received ≥1 dose of MenACWY
68.1% of all adolescents received ≥1 dose of HPV
50.8% received ≥2 doses of MenACWY
17.2% received ≥1 dose of MenB
Coverage for 2nd dose in a 2-dose series at age 16 years assumed to be 50% of that of the 1st dose 3 and 70% at age 11 years.

### Results

With the current coverage levels and a vaccination schedule that requires a total of 4 injections, the MenACWY and MenB vaccines are estimated to avert 178 IMD cases over 10 years, compared to the hypothetical of no vaccination at all. Replacing MenACWY or/and MenB vaccinations with Penta eliminates at least 1 injection; assuming the immunization coverage at age 16 years remains similar to the current MenACWY vaccine coverage, the Penta vaccine was estimated to avert a similar or higher number of IMD cases (FIG. 3).

Two doses of Penta at age 11 years and 1 dose of Penta at age 16 years (scenarios 10-12) could prevent the most cases (up to 282), compared with other vaccination schedule with comparable coverage rate.

One dose of Penta at age 11 years and 2 doses of Penta at age 16 years (scenarios 4-6) could prevent up to 251 cases.

With a slightly higher coverage of the 2nd dose of Penta than the current MenB coverage rate at age 16 years, a similar number of IMD cases could be prevented with one dose of MenACWY vaccine at age 11 years and 2 doses of Penta at age 16 years (scenario 3) or 2 doses of Penta at age 16 (scenario 8, see FIG. 1).

### Conclusion

The disease impact of a vaccination strategy is directly related to the level of coverage obtained. Replacing one or more MenACWY or MenB vaccine doses with Penta could further reduce IMD caused by all 5 meningococcal serogroups;

Reduce number of vaccine administrations for adolescents;

Potentially improve compliance with ACIP recommendations, reduce medical visits and the costs of public health responses to individual IMD cases.

Invasive meningococcal disease (IMD) caused by Neisseria meningitidis is an uncommon, rapidly progressing, and potentially deadly infection with highest incidence observed in infant and adolescent age groups. Serogroups A, B, C, W, and Y account for 94% of disease globally; in the United States, most disease is caused by serogroups B, C, and Y. According to 2018 surveillance data from the United States, the incidence of IMD was 0.10 cases per 100,000 population. Serogroup B was predominant among US adolescents and young adults (62% of cases among 16- to 23-year-olds) and also across all age groups (36% of cases).

The US Advisory Committee on Immunization Practices (ACIP) currently recommends 2 types of meningococcal vaccines to help protect healthy adolescents against IMD. The quadrivalent meningococcal serogroups A, C, W, and Y (MenACWY) vaccine is routinely recommended as a primary dose at age 11 to 12 years and a booster dose at age 16 years. Meningococcal serogroup B (MenB) vaccination is recommended for adolescents and young adults aged 16 to 23 years (16-18 years preferred) based on shared clinical decision-making. In 2018, the estimated MenACWY vaccination coverage for adolescents aged 13 to 17 years was 86.6% for ≥1 dose and 50.8% for ≥2 doses. In contrast, only 17.2% of 17-year-olds received ≥1 dose of a MenB vaccine, and fewer than 50% of these individuals completed the multidose vaccination series. These data suggest that many adolescents in the United States are not fully protected against meningococcal disease.

A single vaccine that can help protect against meningococcal disease caused by all 5 serogroups (ie, a MenABCWY pentavalent vaccine) instead of 2 separate MenB and MenACWY vaccines with different vaccination schedules could simplify immunization, reduce the number of injections required, and potentially improve vaccination coverage. We developed a model to evaluate the public health impact of assorted meningococcal immunization programs using a pentavalent MenABCWY vaccine.

### 2.0 Methods

### 2.1 Model Description

A population-based dynamic model was developed to estimate the expected number of IMD cases averted over a 10-year period in the United States. The model structure is similar to what has been previously described in full elsewhere. The population was stratified into 101 single-year age bands, and individuals in each age band transitioned to the next age band in the following year. Meningococcal carriage was the principal source of infectious disease transmission and was the primary consideration in the model calculations. Meningococcal carriage and transmission were modeled by stratifying the population into 10 mutually exclusive age groups (0-5 months, 6-12 months, 1 year, 2-4 years, 5-9 years, 10-14 years, 15-19 years, 20-24 years, 25-59 years, and >60 years). Each age group was characterized by a proportion of individuals who were carriers of meningococcal serogroups A, B, C, W, and Y and had age-specific probabilities of developing IMD and transmitting the bacteria within their age group or to other age groups. The proportion of meningococcal carriers in each of the 10 age groups for each year was calculated based on (1) carriage prevalence in the prior year; (2) bacterial transmission and mixing patterns within and among the age groups; (3) number of vaccinated individuals (vaccination coverage); and (4) vaccine efficacy against carriage acquisition. During each year of the 10-year time horizon within the model, proportions of individuals in targeted age groups were estimated to receive MenACWY, MenB, and/or MenABCWY vaccines under 4 different scheduling scenarios. For all vaccination scenarios, the model estimated that individuals who developed IMD either recovered with or without complications, or died.

### 2.2 Model Inputs

Average age-group-based IMD incidence rates for each serogroup were derived from the Centers for Disease Control and Prevention (CDC) Enhanced Meningococcal Disease Surveillance reports from 2015-2017. The MenABCWY vaccine was assumed to provide direct protection against serogroups A, B, C, W, and Y.

Vaccine efficacy assumptions against serogroup B in adolescents receiving 1 or 2 doses of the MenABCWY vaccine were based on a published clinical study for the MenB-FHbp vaccine (Trumenba®, bivalent rLP2086; Pfizer Inc, Philadelphia, PA). In this study, the percentage of subjects with serum bactericidal activity in assays using human complement (hSBA) titers 21:8 (standard correlate of protection is 1:4) at 1 month postvaccination ranged from 23.8% to 67.6% and 69.1% to 100% after 1 or 2 doses of MenB-FHbp, respectively. Based on these data, estimates of 30% and 85% vaccine efficacy against serogroup B were assumed for adolescents receiving 1 or 2 doses of MenABCWY vaccine, respectively.

Vaccine efficacy assumptions against serogroups A, C, W, and Y were based on a review of published immunogenicity and efficacy data from clinical studies of the MenACWY-TT vaccine (Nimenrix®, Pfizer Ltd, Sandwich, UK) in which the percentage of subjects with serum bactericidal activity in assays using rabbit complement (rSBA) or hSBA titers 21:8 at 1 month postvaccination ranged from 81.9% to 97.4% after 1 dose of MenACWY-TT. Based on these data, an estimate of 95% vaccine efficacy was assumed against serogroups A, C, W, and Y.

Indirect protection of nonvaccinated individuals due to reduction of carriage prevalence and transmission was assumed to be 0% for serogroup B and 36.2% for serogroups A, C, W, and Y, both derived from the published literature on MenB and MenACWY vaccines.

The 5-year duration of protection and a fixed 10% annual waning rate for the MenABCWY vaccine against serogroup B shown in were assumed based on considerations of (1) previous published health economic models and (2) clinical data from a phase 3 extension study in adolescents that evaluated persistence of the immune response elicited by the MenB-FHbp vaccine. Results from the clinical study indicated that response rates peaked after primary vaccination, declined over the subsequent 12 months, and then remained stable above baseline through 48 months. At 48 months after primary vaccination, 18.0% to 61.3% of subjects had hSBA titers greater than or equal to the lower limit of quantification (ie, 1:16 or 1:8 depending on strain) across the 4 diverse serogroup B test stains used to assess breadth of protection.

For assessment of the protection of the MenABCWY vaccine against serogroups A, C, W and Y (Table 2), the 5-year duration of direct protection and 10% annual waning rate were conservative assumptions based on clinical data from studies that evaluated persistence of the immune response elicited by MenACWY-TT in adolescents and adults aged 11 to 55 years through 10 years after primary vaccination. At year 10, 70.2% to 90.7% of vaccinated subjects had rSBA titers 21:8 across serogroups A, C, Wand Y compared with 99.7% to 100% at 1 month postvaccination. Currently no data are available regarding the duration of indirect protection and waning rates for licensed MenACWY vaccines; the model therefore assumed waning rates for MenACWY were equal to MenABCWY.

### 2.3 Vaccination Scenarios and Sensitivity Analyses

The vaccination scenarios were built based on the existing adolescent meningococcal vaccination platform (ie, at age 11 and 16 years) in the United States. Four primary vaccination schedules were examined and compared with the current schedule: (1) 1 dose of MenACWY vaccine at age 11 years and 2 doses of MenABCWY vaccine at age 16 years; (2) 1 dose of MenABCWY vaccine at age 11 years and 2 doses of MenABCWY vaccine at age 16 years; (3) 2 doses of MenABCWY vaccine at age 16 years only; and (4) 2 doses of MenABCWY vaccine at age 11 years and 1 dose at age 16 years.

The assumptions of vaccination coverage for each primary schedule were taken from observed adolescent vaccination coverage, vaccination age, and number of doses required as reported in the 2018 National Immunization Survey-Teen (NIS-Teen). In line with the trends observed in the NIS-Teen survey, vaccination coverage among adolescents aged 11 years was assumed to be higher than at age 16 years, and the compliance (ie completion of the recommended dosing series) with a 2-dose series at age 11 years was assumed to be higher than compliance with a 2-dose series at age 16 years.

For the base case analysis, vaccination coverage for the first dose at age 11 years was assumed to be the same as the overall coverage of the primary dose of MenACWY vaccine reported in 2018 (86.6%), and vaccination coverage for the first dose at age 16 years was assumed to be the same as the coverage of the MenACWY booster dose (50.8%). It was assumed based on these data that 80% of adolescents aged 11 years who received the first dose of MenABCWY would complete the 2-dose series, whereas compliance with a 2-dose series at age 16 years was assumed to be 50% based on available information on MenB vaccine series completion. Sensitivity analyses were performed for each of the 4 meningococcal vaccine administration schedules using levels of adolescent vaccine coverage at age 16 years reported in 2018. The highest coverage assumed was the same as that for ≥1 dose of human papillomavirus (HPV) vaccine (68.1%) at age 11-12 years, and the lowest coverage assumed was the same as that for ≥1 dose of MenB vaccine (17.2%) at age 16 years. After considering the primary dosing schedules and vaccination coverage estimates, a total of 13 different scenarios were assessed.

### 3.0 Results

With the current vaccination schedule and reported vaccination coverage in 2018 (MenACWY, 86.6% at age 11 years and 50.8% at age 16 years; MenB, 17.2% at age 16 years), vaccination with 2 doses each of MenACWY and MenB vaccines, for a total of 4 injections between 11 and 16 years of age, could potentially avert 165 cases of IMD over the next 10 years compared with no meningococcal vaccination (current scenario). Under this scenario, 19 serogroup B cases (11.5% of the total preventable IMD cases) are estimated to be prevented over the next 10 years. Replacing either MenACWY and/or MenB vaccines with a pentavalent MenABCWY vaccine would eliminate 1 or 2 injections, depending on the vaccination schedule, and potentially avert a higher number of IMD cases (scenarios 1, 2, 4, 5, 7, 8, 10, and 11). This is assuming that MenABCWY vaccination coverage at 16 years of age remains similar to 2018 MenACWY vaccination coverage (50.8%; scenarios 1, 4, 7, and 10) or perhaps rises to the slightly higher HPV vaccination coverage observed at age 11 to 12 years (68.1%; scenarios 2, 5, 8, and 11).

However, assuming MenABCWY vaccination coverage at age 16 years is the same as for the current 2-dose MenB vaccination schedule at age 16 years (17.2%), a MenABCWY regimen of 1 dose at age 11 years and 2 doses at age 16 years (ie, similar to the current schedule) would prevent fewer IMD cases (n=137) compared with the current vaccination schedule (FIG. 2, scenario 6). These results are mainly driven by the assumption of lower MenACWY vaccination coverage than is currently reported at age 16 years, leading to a lower estimated number of serogroups A, C, W, and Y cases averted (n=89; 65.0%) compared with the current schedule.

### 3.1 Base Case Vaccination Coverage Assumptions

In all base case vaccination scenarios (scenarios 1, 4, 7, and 10), assuming MenABCWY vaccination coverage is the same as current coverage for the MenACWY vaccine at age 11 years (86.6%) or age 16 years (50.8%), replacing either the MenACWY and/or MenB vaccine with a pentavalent MenABCWY vaccine would avert a greater number of IMD cases than the current schedule (range, 189-256 IMD cases averted, depending on schedule). The higher number of total IMD cases averted compared with the current vaccination schedule is mainly driven by the greater number of serogroup B cases prevented (range, 55-111 serogroup B cases). Among all base case vaccination scenarios assessed, disease prevention would be maximized by administering 2 doses of MenABCWY vaccine at age 11 years and 1 dose at age 16 years (scenario 10; 256 cases averted [111 serogroup B; 146 serogroups A, C, W, and Y]).

### 3.2 Sensitivity Analysis Based on Alternative Vaccination Coverages

If MenABCWY vaccination coverage at age 16 years rises to the levels observed for the HPV vaccine at 11 to 12 years of age (68.1%), the greatest impact on meningococcal disease prevention would be provided by 2 doses of MenABCWY vaccine at age 11 and 1 dose at age 16 (scenario 11; 299 total cases averted). This schedule would also prevent the greatest number and percentage of serogroup B cases out of the total preventable cases over a 10-year period (140 serogroup B cases averted [46.8%]). The next best regimen was 1 dose of MenABCWY vaccine at age 11 and 2 doses of MenABCWY vaccine at age 16 (scenario 5; 263 cases averted; 103 serogroup B; 159 serogroups A, C, W, and Y). Additionally, a similar number of cases would be averted with or without including the currently recommended MenACWY dose at age 11 years (scenarios 2 [234 cases averted; 74 serogroup B; 159 serogroups A, C, W, and Y] and 8 [220 cases averted; 74 serogroup B; 146 serogroups A, C, W, and Y]). In contrast, regimens where MenABCWY vaccination coverage was assumed to be the same as for the current 2-dose MenB vaccination schedule at age 16 (17.2% for ≥1 dose) led to far fewer estimated IMD cases prevented, mainly due to fewer serogroup B cases averted (scenarios 3, 6, 9, and 12). Under this vaccination coverage assumption, the lowest number of total IMD cases prevented would be through a regimen of 2 MenABCWY vaccines at age 16 (scenario 9, 93 total cases averted [19 serogroup B; 74 serogroups A, C, W, and Y]).

### 4.0 Discussion

This is the first study to our knowledge that models the impact of a pentavalent MenABCWY vaccine to protect against meningococcal disease caused by the 5 most prevalent disease-causing serogroups (ie, serogroups A, B, C, W, and Y) in the context of the US adolescent meningococcal immunization platform. Globally, various monovalent, bivalent, or quadrivalent meningococcal vaccine formulations that target different combinations of these 5 serogroups are used to help protect against meningococcal disease. In several countries, ongoing surveillance efforts have detected changes in circulating disease-causing meningococcal serogroups; these epidemiological shifts prompted changes to some national vaccination strategies to include MenACWY vaccines for comprehensive protection against IMD. Deploying a MenABCWY vaccine would potentially protect against the 94% of IMD cases worldwide estimated to be caused by these 5 serogroups.

In the United States, MenACWY vaccination for adolescents has been recommended since 2005, and MenB vaccination recommendations were put into effect in 2015. A single pentavalent MenABCWY vaccine could simplify immunization schedules by eliminating the need for multiple injections using 2 different vaccines at different ages, potentially improving vaccination coverage and enhancing protection against most prevalent disease-causing serogroups. Based on the current schedule and vaccination coverage for MenACWY and MenB vaccines in the United States, our model estimates that vaccination with both MenACWY and MenB vaccines could potentially avert 165 cases of IMD over 10 years compared with no vaccination. Assuming MenABCWY vaccination coverage is similar to current MenACWY vaccination coverage noted by the CDC NIS-Teen survey from 2018, our model estimates that replacing one or more MenACWY or MenB vaccine doses with a MenABCWY vaccine would lead to as many as 256 IMD cases averted among US adolescents while simultaneously reducing the recommended number of vaccine injections. In fact, most of the scenarios examined in this study demonstrate added benefit of a single MenABCWY vaccine in terms of greater number of cases averted compared with the current schedule.

Adolescent immunization delivery is challenging, in part because rates of preventative well-visits—when immunizations typically occur—decline steadily after 16 years of age, and when combined with the transition from pediatricians to medical providers who are typically less involved in adolescent immunizations, may contribute to suboptimal protection against disease in this age-group. An age-based platform for MenABCWY vaccination would support and catalyze adolescent immunization by enabling vaccine administration at ages where adolescents are more likely to receive and comply with multidose regimens and reduce the number of injections and visits. Importantly, immunization of adolescents with MenACWY conjugate vaccines and recombinant protein MenB vaccines both elicit protective immune responses following primary vaccination and robust responses following a booster dose. Together, these data not only support to the existing US adolescent MenACWY and MenB immunization platform, but also lend support to a flexible MenABCWY vaccination schedule where adolescents can start the vaccination series anywhere between the ages of 11 and 16 years and maintain protection throughout the period of highest risk.

Studies have shown that socioeconomic status, education, and race also play a role in vaccination awareness, access, and utilization, and series-completion rates. As such, a MenABCWY vaccine could help reduce these disparities by simplifying meningococcal vaccination recommendations, thereby reducing vaccine access issues, and eliminating confusion surrounding existing MenB and MenACWY vaccine recommendations. Moreover, combination vaccines in general have been shown to improve vaccination coverage among a variety of age groups.

### Example 18: Breadth of the Human Immune Response to TRUMENBA: Summary of fHBP Variants Expressed by MenB Strains that are Susceptible in the hSBA

Introduction & Aims: TRUMENBA (bivalent rLP2086), a vaccine for the prevention of Neisseria meningitis serogroup B (MenB) disease, includes two protein antigens, variants of meningococcal factor H binding protein (fHBP). fHBP exists as two subfamilies, A and B. Within each subfamily several hundred unique fHBP variants have been identified. Despite this sequence diversity, a vaccine containing one protein from each subfamily was demonstrated to induce broad coverage across MenB strains that represent the diversity of fHBP variants. Licensure was based on the ability of the vaccine to elicit antibodies that initiate complement-mediated killing of invasive MenB strains in a serum bactericidal assay using human complement (hSBA). Due to the endemic nature of meningococcal disease, it is not possible to predict which fHBP variants individuals may be exposed to. For this reason we have continued to explore the coverage conferred by TRUMENBA and present here additional evidence to illustrate the breadth of immune coverage.

Materials & Methods: MenB invasive strains (n=109) were selected to confirm TRUMENBA breadth of coverage. The strains encoded 22 and 16 unique subfamily A and subfamily B fHBP variants, respectively. The expression of fHBP at the bacterial surface was determined using the flow cytometric MEningococcal Antigen SURface Expression (MEASURE) assay. Exploratory hSBAs were performed using pre- and post-vaccination sera (subject-matched) from young adults. A strain was considered susceptible to TRUMENBA immune sera if a 4-fold rise in the hSBA titer was achieved between the pre- and post-vaccination serum samples.

Results: Of the 109 strains, 87 (nearly 80%) were susceptible to TRUMENBA immune serum in hSBAs. This included strains expressing fHBP variants A02, A28, A42, A63, A76, B05, B07, B08, B13, B52 and B107, in addition to variants that had been reported previously. The majority of strains that could not be killed had fHBP expression levels that were below the level considered sufficient to initiate bactericidal killing in an hSBA. See FIG. 3, Table 1, Table 2, and Table 3.

TABLE 20

| | fHBP | fHBP Expression | Susceptible |
|---|---|---|---|
| Strain ID | Variant | (MFI) | in hSBA¹ |
| PMB3693 | A02 | 13157 | + |
| PMB876 | A28 | 4193 | + |
| PMB3106 | A42 | 1614 | + |
| PMB2871 | A63 | 10818 | + |
| PMB1606 | A76 | 11331 | + |
| PMB2627 | B05 | 2916 | + |
| PMB2219 | B07 | 1350 | + |
| PMB1610 | B08 | 1561 | + |
| PMB1486 | B13 | 1850 | + |
| PMB2466 | B52 | 8734 | + |
| PMB891 | B107 | 11125 | + |

TABLE 21
fHBP variants expressed by MenB strains that were killed
with TRUMENBA immune sera in hSBAs (% amino acid sequence
identity with A05 (SEQ ID NO: 1) and B01 (SEQ ID NO: 2))

| Subfamily A | Subfamily B |
|---|---|
| A02 (94.3) | B02 (92.0) |
| A04 (96.6) | B03 (90.8) |
| A05 (vaccine antigen) | B05 (87.7) |
| A06 (96.2) | B07 (87.3) |
| A07 (85.4) | B08 (87.7) |
| A12 (85.4) | B09 (88.1) |
| A15 (85.1) | B107 (89.7) |
| A17 (88.1) | B13 (86.9) |
| A19 (88.1) | B15 (86.5) |
| A22 (88.9) | B16 (86.2) |
| A26 (85.8) | B24 (86.2) |
| A28 (96.9) | B44 (91.6) |
| A42 (91.6) | B52 (91.9) |
| A56 (98.1) | |
| A63 (96.6) | |
| A76 (95.0) | |

TABLE 22
fHBP variants* expressed by strains at <1000 MFI
and were not killed with TRUMENBA immune sera in
hSBAs (% amino acid sequence identity with vaccine
antigens A05 (SEQ ID NO: 1) and B01 (SEQ ID NO: 2))

| Subfamily A | Subfamily B |
|---|---|
| A08 (85.9) | B10 (88.1) |
| A10 (85.4) | B91 (90.4) |
| A20 (87.7) | |
| A40 (85.1) | |
| A52 (96.6) | |

Conclusion: The hSBA is recognized as the surrogate of efficacy for meningococcal vaccines. Assay complexity prevents demonstration of the bactericidal activity of TRUMENBA immune sera against MenB strains that express each of the hundreds of unique fHBP sequence variants. To illustrate the breadth of immune coverage conferred by TRUMENBA, we show that MenB strains expressing additional diverse fHBP variants can be killed in hSBAs despite being heterologous to the vaccine antigens.

### Example 19: Selection of Diverse Strains to Assess Broad Coverage of the Bivalent FHbp Meningococcal B Vaccine

Although transmission of Neisseria meningitidis usually results in asymptomatic colonization of the upper respiratory tract, in some individuals, bacteremia and invasive meningococcal disease (IMD) occur. IMD commonly presents as meningitis and/or septicemia; pneumonia, septic arthritis, epiglottitis, and otitis media are less frequently observed. A high case fatality rate is associated with IMD (10%-15%), and approximately 20% of survivors have serious life-long sequelae such as limb amputation, hearing loss, and neurologic impairment.

Nearly all meningococcal disease worldwide is caused by 6 of the 12 characterized meningococcal serogroups (ie, A, B, C, W, X, and Y). Effective vaccines based on capsular polysaccharides have been developed for serogroups A, C, W, and Y. However, immunogenicity of the MenB polysaccharide is poor because of similarity to polysialic acid structures present on human neuronal cells. During recent years, meningococcal serogroup B (MenB) in particular has been associated with a large proportion of IMD in Europe, the United States, Canada, Australia, and New Zealand. Although vaccines based on outer membrane vesicles (OMVs) have been successfully used to control epidemics caused by a single MenB outbreak strain, the generated immune response is predominantly against the highly variable porin A protein (PorA). Therefore, effectiveness is generally limited to the target strain. Consequently, surface-exposed proteins capable of inducing protective bactericidal antibodies across diverse MenB strains have been sought for the development of a broadly effective MenB vaccine.

Factor H binding protein (FHbp; also known as LP2086 and GNA1870), a conserved surface-exposed lipoprotein expressed on nearly all strains of MenB, was identified as such a target. Based on amino acid sequence, FHbp variants segregate into 2 immunologically distinct subfamilies (termed subfamily A and subfamily B); each MenB strain expresses a single subfamily variant (see FIG. 1A).

MenB-FHbp (TRUMENBA®, bivalent rLP2086; Pfizer Inc, Philadelphia, PA, USA) is a bivalent, recombinant protein MenB vaccine composed of equal amounts of 2 recombinant lipidated FHbp antigens, one from subfamily A (variant A05) and the other from subfamily B (variant B01). Importantly, it is predicted that this combination of FHbp variants is capable of providing protection against diverse MenB strains. MenB-FHbp has been approved for the prevention of IMD in several countries and regions, including the United States, Canada, Europe, and Australia. Another MenB vaccine, MenB-4C (Bexsero®, 4CMenB; GlaxoSmithKline Vaccines, Srl, Siena, Italy), also has a recombinant FHbp component (nonlipidated variant 1.1 from subfamily B) as well as 2 other recombinant protein antigens and an OMV. Thus, MenB-4C is different from MenB-FHbp, which contains two variants of a single antigen to afford broad coverage.

The serum bactericidal assay using human complement (hSBA) measures complement-dependent, antibody-mediated lysis of meningococcal bacteria. An hSBA titer is defined as the highest serum dilution killing ≥50% of assay bacteria; an hSBA titer 1:4 is the accepted correlate of protection against meningococcal disease, and hSBA response rates based on this correlate have been used as surrogates for meningococcal vaccine efficacy. The SBA response rate has been specifically correlated with natural protection for the serogroup C and A polysaccharide vaccines. Because serogroup-specific polysaccharides are not variable, a single strain from each serogroup was sufficient to infer broad vaccine coverage. MenB OMV vaccines are also efficacious and vaccine-elicited hSBA titers correlated with protection against the target strain causing the epidemic. Accurately predicting strain coverage of protein-based vaccines is more complex using hSBA than for vaccines targeting capsular polysaccharides, given that protein sequence diversity and variability in expression levels differ among the different meningococcal disease strains. For example, (PorA is the predominant target for serum bactericidal antibodies conferring protection after OMV vaccine immunization. PorA is a cell surface porin whose small cell surface exposed region has a high degree of sequence diversity. It has been estimated that protective immunity would need to be demonstrated with strains expressing 20 different PorA serosubtypes to protect against approximately 80% of sporadic MenB disease-causing strains in the United States. Historically, OMV vaccines have contained 1 PorA and have not demonstrated protection against strains with PorA sequences that are heterologous in amino acid sequence compared with the vaccine antigen. Therefore, selection of representative test strains to demonstrate that vaccine-elicited antibodies can be effective against a meningococcal disease strain is of paramount importance for protein-based vaccines.

Immune sera elicited by MenB-FHbp in preclinical and early clinical studies demonstrated broad bactericidal antibodies that could kill diverse MenB strains containing FHbp subfamily A and B variants heterologous to the vaccine FHbp variants A05 and B01. In an early assessment of the potential breadth of MenB-FHbp coverage, 100 MenB isolates with diverse FHbp variants, geographic origins, and genetic backgrounds were tested in hSBAs using MenB-FHbp immune rabbit serum. Of the 100 strains tested, 87 were killed in these hSBAs. Analysis of the 13 strains that were not killed suggested that the threshold FHbp surface expression level on a given MenB strain affected the hSBA response. A threshold FHbp surface expression level was subsequently determined, above which isolates were predictably killed in hSBA. Additional investigations of potential factors determining strain susceptibility found that killing was largely independent of FHbp sequence variant, multilocus sequence type, or PorA subtype.

To select strains with broad antigenic and epidemiologic diversity for clinical testing, over 1200 invasive MenB disease isolates were collected from laboratories and health agencies in the United States and Europe to represent the prevalence of MenB isolates that were contemporary at the time of collection; all strains contained the FHbp gene. An unbiased approach was used to select 4 antigenically and epidemiologically diverse representative test strains for use in MenB-FHbp immunogenicity studies. Selection criteria included expression of FHbp variants heterologous to the vaccine antigens and adequately reflecting the diversity of FHbp in MenB disease isolates, low to medium FHbp surface expression levels, and low baseline hSBA seropositivity rates. These 4 primary MenB test strains express FHbp variants from both FHbp subfamilies (strain [variant]: PMB2001 [A22], PMB80 [A56], PMB2707 [B24], and PMB2948 [B44]; see FIG. 1A).

To supplement immunogenicity data generated using the 4 primary MenB test strains and to demonstrate that immune responses against the 4 primary MenB test strains are predictive of immune responses against the diversity of FHbp variants expressed by MenB disease-causing isolates, hSBAs using 10 additional test strains were developed. The 10 additional test strains were selected to include prevalent FHbp variants found in MenB disease-causing strains in the United States and Europe. Here, we (i) describe the strategy and criteria used to select the 10 additional test strains, and (ii) present data demonstrating that the immune responses measured by hSBA using the 4 primary MenB strains are predictive of the responses obtained using 10 additional test strains, which further demonstrate and support the broad coverage of the immune response elicited by MenB-FHbp.

### Results

### Sources and Selection Criteria for the Additional MenB Test Strains

Nine of the 10 additional MenB test strains were obtained from a collection of 1263 invasive disease-causing MenB strains (the MenB isolate collection). For the MenB isolate collection, US strains were from the Active Bacterial Core Surveillance sites (2000-2005), covering approximately 13% of the population. European isolates (2001-2006) were from the public health laboratories of Norway, France, Czech Republic and the Health Protection Agency in Manchester (which covers England, Wales, and Northern Ireland) and were collected systematically (every seventh or eighth isolate was included by order received at the country's reference laboratory) and represented approximately 13% of invasive MenB isolates during the period. The strains expressing FHbp variant A07 were obtained from an extension of the MenB isolate collection that included an additional 551 disease-causing MenB strains from Spain and Germany (n=1814). The extended MenB isolate collection was used as A07-expressing strains in the MenB isolate collection were not suitable because of the low surface expression of FHbp on these strains, high baseline seropositivity, and lack of readily available source of complement.

The criteria used to select the additional MenB test strains were (i) FHbp variant prevalence among MenB disease-causing strains in the United States and/or Europe, (ii) the FHbp variant needed to be different from those expressed by MenB primary test strains, (iii) in vitro FHbp expression levels at or below median levels for the respective FHbp variant group to ensure that the strain was representative of the variant group it belonged to, (iv) technical compatibility in the hSBA, and (v) being considered a predominant clonal complex for the variant group (if a predominant complex existed). Strains meeting these criteria also needed to be technically compatible in the hSBA, including adequate availability of suitable human complement lots (FIG. 2). Strains in each FHbp variant group with expression levels below the cutoff level (ie, at or below median levels for the respective FHbp variant group) were randomly selected, with the first strains within an FHbp variant group meeting the required genetic, phenotypic, and hSBA development criteria becoming the additional MenB test strains. An exception to this methodology was made for the strain expressing FHbp variant B03, which was selected in collaboration with and using guidance provided by the US FDA based on its previous use in a phase 2 study.

### Characteristics of the Additional MenB Test Strains

The 10 additional selected MenB test strains express FHbp variants A06, A07, A12, A15, A19, A29, B03, B09, B15, and B16 which differ from the ones in the 4 primary test strains (A22, A56, B24, B44) and have different sequences compared to the vaccine antigens (Table 23). The specific variants expressed by the 4 primary test strains are present in 42.0% (530/1263) of disease-causing isolates in the MenB isolate collection, and the specific variants expressed by the 10 additional test strains are present in an additional, non-overlapping 38.8% (490/1263) of disease-causing isolates in the MenB isolate collection (FIG. 1B).

TABLE 23
Characteristics of the 4 Primary and 10 Additional MenB Test Strains

| | | | | | | |
|---|---|---|---|---|---|---|
| | | Percentage | | FHbp Variant | | |
| | | Identity to | Strain | Group MEASURE | | |
| | FHbp | Vaccine | MEASURE | MFI Median^{b} | Clonal | Country of |
| Strain | Variant | Component | MFI^{a} (±1 SD) | (±1 SD) | Complex | Isolation |

Primary Strains

| | | | | | | |
|---|---|---|---|---|---|---|
| PMB80 | A22 | 88.9 | 3127 (2440, 4007) | 2502 (1952, 3207) | CC41/44 | United States |
| PMB2001 | A56 | 98.1 | 5002 (3903, 6410) | 5002^{c} | CC213 | France |
| PMB2948 | B24 | 86.2 | 6967 (5436, 8929) | 8457 (6599, 10,839) | CC32 | France |
| PMB2707 | B44 | 91.6 | 11,283 (8804, 14,461) | 14,753 (11,511, 18,907) | CC269 | United Kingdom |

Additional Strains

| | | | | | | |
|---|---|---|---|---|---|---|
| PMB3010 | A06 | 96.2 | 3370 (2629, 4319) | 3088 (2410, 3958) | CC461 | United Kingdom |
| PMB3040 | A07 | 85.4 | 1379 (1076, 1767) | 1100 (858, 1409) | CC162 | Germany |
| PMB824 | A12 | 85.4 | 2540 (1982, 3255) | 2467 (1925, 3161) | CC35 | United States |
| PMB1672 | A15 | 85.1 | 2995 (2337, 3838) | 2904 (2266, 3721) | CC103 | France |
| PMB1989 | A19 | 88.1 | 1934 (1509, 2479) | 1759 (1372, 2254) | CC8 | United Kingdom |
| PMB3175 | A29 | 93.1 | 3839 (2995, 4920) | 5994 (4677, 7682) | CC32 | United States |
| PMB1256 | B03 | 90.8 | 3976 (3102, 5096) | 2935 (2290, 3762) | CC41/44 | United Kingdom |
| PMB866 | B09 | 88.1 | 2089 (1630, 2677) | 2275 (1775, 2916) | CC269 | United Kingdom |
| PMB431 | B15 | 86.5 | 3785 (2953, 4851) | 4822 (3763, 6180) | CC41/44 | United States |
| PMB648 | B16 | 86.2 | 2347 (1831, 3008) | 1996 (1557, 2558) | CC41/44 | United Kingdom |

FHbp = factor H binding protein;
MenB = Neisseria meningitidis serogroup B;
MFI = mean fluorescence intensity;
SBA = serum bactericidal assay.
^{a}MFI ± 1 SD from MEASURE assay.
^{b}Based on the MenB SBA isolate collection (n = 1263), except for variant group A07, which was calculated from the extended MenB SBA isolate collection (n = 1814). Strains in each FHbp variant group with expression levels at or below median levels for the respective FHbp variant group were randomly selected. The cutoff level adopted for each FHbp variant group was the observed median MFI plus 1 SD, using the precision estimate of 25.2% relative SD.
^{c}There is only one strain expressing A56; thus, no SD values are included.

Immunogenicity Analysis: Subjects with hSBA Titer ≥LLOQ for the 10 Additional Strains

The 4 primary strains were used to assess serological responses after 2 or 3 doses of MenB-FHbp in subjects participating in 2 pivotal phase 3 studies in adolescents and young adults. Serological responses to the 10 additional hSBA strains were assessed in a subgroup of the study subjects. The majority of subjects had hSBAs ≥lower limit of quantitation (LLOQ; ie, hSBA titer equal to 1:8 or 1:16, depending on strain) 1 month after dose 2 and 1 month after dose 3 for each of the primary (64.0%-99.1% and 87.1%-99.5%, respectively) and the 10 additional MenB test strains (51.6%-100.0% and 71.3%-99.3%, respectively) (Table 24). For the primary and additional MenB test strains, a substantial increase from baseline in the proportion of subjects achieving an hSBA titer LLOQ was observed among MenB-FHbp recipients (0, 2, 6 month schedule) after the second MenB-FHbp dose, with additional increases after the third dose.

TABLE 24
Subjects With hSBA Titers ≥ LLOQ (1:8 or 1:16) for Primary and Additional MenB
Test Strains

| | % (95% CI) | | | | | |
|---|---|---|---|---|---|---|
| | [n] | | | | | |
| | Adolescents^{a} | | | Young Adults^{a} | | |
| | | 1 Month | 1 Month | | 1 Month | 1 Month |
| FHbp | | After | After | | After | After |
| Variant | Prevaccination | Dose 2 | Dose 3 | Prevaccination | Dose 2 | Dose 3 |
| Primary | | | | | | |
| strain | | | | | | |
| A22 | 33.2 (30.6, 35.9) | 94.3 | 97.8 | 33.6 (31.3, 35.9) | 84.7 | 93.5 |
| | [1238] | (92.9, 95.5) | (96.8, 98.5) | [1704] | (82.9, 86.4) | (92.2, 94.6) |
| | | [1263] | [1266] | | [1697] | [1714] |
| A56 | 27.5 (24.9, 30.2) | 99.1 | 99.5 | 32.2 (29.9, 34.5) | 97.4 | 99.4 |
| | [1135] | (98.4, 99.5) | (98.9, 99.8) | [1657] | (96.5, 98.1) | (98.9, 9 9.7) |
| | | [1222] | [1229] | | [1701] | [1708] |
| B24 | 6.4 (5.1, 7.9) | 66.4 | 87.1 | 33.1 (30.9, 35.4) | 86.5 | 95.1 |
| | [1264] | (63.6, 69.0) | (85.1, 88.9) | [1696] | (84.7, 88.1) | (93.9, 96.0) |
| | | [1216] | [1250] | | [1685] | [1702] |
| B44 | 3.6 (2.6, 4.8) | 64.0 | 89.3 | 11.0 (9.6, 12.6) | 68.3 | 87.4 |
| | [1230] | (61.3, 66.8) | (87.4, 90.9) | [1716] | (66.1, 70.6) | (85.8, 89.0) |
| | | [1204] | [1210] | | [1693] | [1703] |
| Additional | | | | | | |
| strain | | | | | | |
| A06 | 9.4 (6.2, 13.5) | 84.0 | 95.7 | 16.0 (11.9, 20.9) | 77.8 | 92.0 |
| | [277] | (75.0, 90.8) | (92.6, 97.8) | [275] | (67.8, 85.9) | (88.1, 94.9) |
| | | [79] | [280] | | [90] | [275] |
| A07 | 43.1 (37.1, 49.3) | 93.8 | 96.4 | 55.8 (49.7, 61.8) | 97.9 | 95.7 |
| | [269] | (86.9, 97.7) | (93.5, 98.3) | [274] | (92.6, 99.7) | (92.6, 97.7) |
| | | [90] | [280] | | [95] | [277] |
| A12 | 3.9 (2.0, 6.9) | 67.4 | 75.1 | 5.0 (2.8, 8.3) | 57.6 | 71.3 |
| | [280] | (57.0, 76.6) | (69.6, 80.1) | [278] | (46.9, 67.9) | (65.5, 76.5) |
| | | [64] | [277] | | [92] | [275] |
| A15 | 20.7 (16.1, 26.1) | 65.6 | 87.2 | 37.3 (31.6, 43.2) | 83.2 | 91.8 |
| | [270] | (55.0, 75.1) | (82.6, 91.0) | [279] | (74.1, 90.1) | (87.9, 94.7) |
| | | [61] | [266] | | [95] | [279] |
| A19 | 11.3 (7.8, 15.7) | 84.5 | 92.7 | 28.8 (23.5, 34.5) | 87.4 | 95.8 |
| | [274] | (75.8, 91.1) | (89.0, 95.5) | [278] | (79.0, 93.3) | (92.7, 97.8) |
| | | [82] | [275] | | [95] | [284] |
| A29 | 17.5 (13.1, 22.5) | 100.0 | 98.6 | 31.1 (25.7, 36.9) | 96.8 | 99.3 |
| | [269] | (96.3, 100.0) | (96.4, 99.6) | [280] | (91.0, 99.3) | (97.5, 99.9) |
| | | [97] | [278] | | [95] | [283] |
| B03 | 4.3 (2.2, 7.4) | 61.1 | 92.5 | 11.2 (7.7, 15.5) | 57.9 | 86.4 |
| | [280] | (50.3, 71.2) | (88.7, 95.3) | [277] | (47.3, 68.0) | (81.8, 90.3) |
| | | [55] | [279] | | [95] | [273] |
| B09 | 15.2 (11.2, 19.9) | 76.3 | 86.2 | 23.5 (18.6, 28.9) | 65.3 | 77.0 |
| | [277] | (66.4, 84.5) | (81.6, 90.1) | [277] | (54.8, 74.7) | (71.6, 81.9) |
| | | [71] | [276] | | [95] | [274] |
| B15 | 28.7 (23.5, 34.5) | 96.8 | 98.2 | 43.8 (37.8, 49.9) | 86.5 | 96.7 |
| | [275] | (90.9, 99.3) | (95.9, 99.4) | [274] | (78.0, 92.6) | (93.9, 98.5) |
| | | [90] | [281] | | [96] | [276] |
| B16 | 7.6 (4.8, 11.4) | 61.6 | 81.7 | 21.9 (17.1, 27.3) | 51.6 | 78.0 |
| | [276] | (50.5, 71.9) | (76.6, 86.0) | [270] | (41.1, 62.0) | (72.6, 82.8) |
| | | [53] | [278] | | [95] | [273] |

FHbp = factor H binding protein;
hSBA = serum bactericidal assay using human complement;
LLOQ = lower limit of quantitation;
MenB = Neisseria meningitidis serogroup B.
Observed proportions of subjects were summarized with exact 2-sided 95% CIs using the Clopper-Pearson method.
LLOQ = 1:16 for A06, A12, A19 and A22;
LLOQ = 1:8 for A07, A15, A29, A56, B03, B09, B15, B16, B24, and B44.
^{a}Evaluable immunogenicity population

### Positive Predictive Values for the Primary and Additional Strains

The relationship between vaccine-induced hSBA responses for the primary MenB test strains and the 10 additional MenB test strains was assessed (Table 25). Within an FHbp subfamily, positive predictive values (PPVs) were greater than 80% for most primary/additional strain pairs 1 month after dose 3. Thus, the immune responses measured in hSBAs using the primary test strains were highly predictive of immune responses for the additional strains within the same subfamily. The PPVs 1 month after dose 2 usually were slightly lower than those observed 1 month after dose 3 and ranged from 61.6% to 100% and 70.0% to 100% for subfamily A and B strain pairs, respectively, across studies. In summary, all PPVs showed high predictability for protective responses when comparing the primary and additional strain hSBA responses.

TABLE 25
Positive Predictive Value of Immune Response to Primary Strain for Immune
Response to Additional Strain following MenB-FHbp Vaccination

| | | % (95% CI)^{a} | | | |
|---|---|---|---|---|---|
| | | [n/N]^{b} | | | |
| FHbp Variant | | Adolescents | | Young Adults | |
| Primary | Additional Test | 1 Month After | 1 Month After | 1 Month After | 1 Month After |
| Test Strain | Strain | Dose 2 | Dose 3 | Dose 2 | Dose 3 |
| A22 | A06 | 89.7 | 96.0 | 87.5 | 94.0 |
| | | (81.27, 95.16) | (92.90, 97.97) | (77.59, 94.12) | (90.26, 96.59) |
| | | [78/87] | [262/273] | [63/72] | [234/249] |
| | A07 | 98.9 | 96.3 | 100.0 | 99.2 |
| | | (93.83, 99.97) | (93.37, 98.23) | (95.20, 100.00) | (97.15, 99.90) |
| | | [87/88] | [263/273] | [75/75] | [249/251] |
| | A12 | 72.7 | 75.9 | 67.6 | 77.9 |
| | | (62.19, 81.68) | (70.37, 80.90) | (55.68, 78.00) | (72.24, 82.91) |
| | | [64/88] | [205/270] | [50/74] | [194/249] |
| | A15 | 70.9 | 89.7 | 92.4 | 93.9 |
| | | (60.14, 80.22) | (85.31, 93.18) | (84.20, 97.16) | (90.27, 96.47) |
| | | [61/86] | [227/253] | [73/79] | [246/262] |
| | A19 | 87.8 | 95.4 | 97.5 | 98.9 |
| | | (79.18, 93.74) | (92.11, 97.60) | (91.15, 99.69) | (96.76, 99.77) |
| | | [79/90] | [249/261] | [77/79] | [265/268] |
| | A29 | 100.0 | 99.6 | 98.7 | 100.0 |
| | | (95.98, 100.00) | (97.91, 99.99) | (93.15, 99.97) | (98.62, 100.00) |
| | | [90/90] | [263/264] | [78/79] | [266/266] |
| A56 | A06 | 84.3 | 96.3 | 83.3 | 93.0 |
| | | (75.02, 91.12) | (93.29, 98.21) | (73.62, 90.58) | (89.23, 95.71) |
| | | [75/89] | [260/270] | [70/84] | [251/270] |
| | A07 | 94.4 | 97.0 | 98.9 | 96.0 |
| | | (87.37, 98.15) | (94.22, 98.71) | (93.90, 99.97) | (92.88, 97.96) |
| | | [84/89] | [261/269] | [88/89] | [261/272] |
| | A12 | 68.2 | 75.6 | 61.6 | 72.2 |
| | | (57.39, 77.71) | (69.94, 80.61) | (50.51, 71.92) | (66.47, 77.48) |
| | | [60/88] | [201/266] | [53/86] | [195/270] |
| | A15 | 64.4 | 89.2 | 84.6 | 92.0 |
| | | (53.38, 74.35) | (84.68, 92.76) | (75.54, 91.33) | (88.10, 94.90) |
| | | [56/87] | [223/250] | [77/91] | [252/274] |
| | A19 | 83.5 | 93.8 | 90.1 | 96.4 |
| | | (74.27, 90.47) | (90.12, 96.41) | (82.05, 95.38) | (93.48, 98.26) |
| | | [76/91] | [242/258] | [82/91] | [268/278] |
| | A29 | 100.0 | 98.9 | 97.8 | 99.6 |
| | | (96.03, 100.00) | (96.68, 99.76) | (92.29, 99.73) | (98.01, 99.99) |
| | | [91/91] | [258/261] | [89/91] | [276/277] |
| B24 | B03 | 80.3 | 97.1 | 75.7 | 89.9 |
| | | (68.16, 89.40) | (94.16, 98.83) | (63.99, 85.17) | (85.53, 93.28) |
| | | [49/61] | [236/243] | [53/70] | [231/257] |
| | B09 | 88.7 | 92.1 | 82.9 | 80.5 |
| | | (78.11, 95.34) | (87.96, 95.19) | (71.97, 90.82) | (75.17, 85.20) |
| | | [55/62] | [222/241] | [58/70] | [207/257] |
| | B15 | 100.0 | 99.6 | 100.0 | 98.8 |
| | | (94.22, 100.00) | (97.75, 99.99) | (94.87, 100.00) | (96.67, 99.76) |
| | | [62/62] | [244/245] | [70/70] | [257/260] |
| | B16 | 82.1 | 86.4 | 70.0 | 81.3 |
| | | (69.60, 91.09) | (81.46, 90.46) | (57.87, 80.38) | (76.01, 85.90) |
| | | [46/56] | [210/243] | [49/70] | [209/257] |
| B44 | B03 | 78.9 | 96.6 | 88.9 | 95.8 |
| | | (66.11, 88.62) | (93.40, 98.52) | (77.37, 95.81) | (92.38, 97.96) |
| | | [45/57] | [227/235] | [48/54] | [227/237] |
| | B09 | 88.3 | 90.1 | 96.4 | 85.9 |
| | | (77.43, 95.18) | (85.50, 93.61) | (87.47, 99.56) | (80.77, 90.09) |
| | | [53/60] | [209/232] | [53/55] | [201/234] |
| | B15 | 100.0 | 99.2 | 100.0 | 98.3 |
| | | (94.04, 100.00) | (96.99, 99.90) | (93.51, 100.00) | (95.74, 99.54) |
| | | [60/60] | [235/237] | [55/55] | [233/237] |
| | B16 | 84.9 | 85.5 | 79.6 | 83.8 |
| | | (72.41, 93.25) | (80.37, 89.77) | (66.47, 89.37) | (78.40, 88.24) |
| | | [45/53] | [201/235] | [43/54] | [196/234] |

hSBA = serum bactericidal assay using human complement; LLOQ = lower limit of quantitation; MenB = Neisseria meningitidis serogroup B.
LLOQ = 1:8 for strains expressing variants A07, A15, A29, A56, B03, B09, B15, B16, B24, and B44; LLOQ = 1:16 for strains expressing variants A06, A12, A19, and A22.
^{a}Exact 2-sided CI based on the observed proportion of subjects using the Clopper-Pearson method.
^{b}N = number of subjects with valid and determinate assay results for both the primary and additional strains with observed hSBA titer ≥LLOQ for the primary strain at 1 month after vaccination 2 and at 1 month after vaccination 3; n = number of subjects with observed hSBA titer ≥LLOQ for the given additional strain at 1 month after vaccination 2 and at 1 month after vaccination 3.

### Discussion

A critical component of the clinical evaluation of the MenB-FHbp vaccine to determine the breadth of protection was the development of hSBAs using test strains with surface protein antigens whose sequence and expression variability are representative of the diversity of MenB disease-causing strains that were contemporary at the time of collection. As described in phase 3 studies in adolescents and young adults, hSBA response data for the 4 primary MenB test strains, all of which express FHbp variants heterologous to the vaccine antigens, strongly suggest that the bivalent MenB-FHbp vaccine provides broad coverage across diverse, disease-causing meningococcal strains. The 10 additional MenB test strains described here provide supportive immunologic data for MenB-FHbp and further confirm the validity of the use of the 4 primary test strains to measure the immune response to MenB-FHbp. As the responses obtained for the 4 primary test strains are predictive of the responses obtained for the additional 10 test strains, the immunological responses obtained by assessing the primary strains in hSBAs are representative of the diversity of strains causing invasive MenB disease.

For the hypothesis test-driven immunogenicity evaluations in licensure studies for MenB-FHbp, an unbiased approach was used to select the 4 primary MenB test strains from panels of disease-causing MenB collected in the United States and Europe. A similar method was used to select the 10 additional MenB hSBA test strains, taking into consideration specific selection criteria to ensure that test strains were representative of the antigenic diversity of MenB isolates. Collectively, the 14 MenB test strains represent the majority of the prevalent meningococcal FHbp, with FHbp variants corresponding to approximately 80% of circulating invasive disease-causing isolates in the United States and Europe.

Positive predictive value analyses were used to determine the association of immune responses, measured by hSBA, among primary and additional test strains expressing FHbps within the same subfamily. All of the PPV analyses showed the high predictability of the protective responses against the primary strain for the protective responses observed against the additional strains. These PPV analyses indicate that the responses observed against the 4 primary MenB test strains are representative of responses to other disease-causing MenB strains that express additional sequence-diverse FHbp variants different from the vaccine antigen variants.

The MenB-FHbp-elicited responses measured by hSBA to the 4 primary and 10 additional MenB test strains were evaluated using sera from individual vaccine recipients. By determining the proportion of vaccinated subjects with functional bactericidal antibodies, assessment of the breadth of MenB-FHbp coverage at the individual level was determined, which is not possible using pooled sera. The 4 primary MenB test strains were selected to represent the diversity of MenB disease-causing IMD and thus support the potential breadth of coverage for MenB-FHbp using hSBA. Responses of individuals with hSBA titers 21:4 are the accepted correlate of protection and a surrogate of meningococcal vaccine efficacy. Thus, the responses provide a comprehensive and biologically predictive assessment of breadth of vaccine coverage. The relevance of the hSBA responses to the 4 primary MenB test strains to describe breadth of vaccine coverage is supported by the demonstration of protective bactericidal responses by MenB-FHbp also observed against diverse and contemporary MenB outbreak strains from Europe and the United States and against non-MenB disease-causing strains (ie, meningococcal serogroups C, Y, W, and X).

Another methodology, the enzyme-linked immunosorbent assay-based Meningococcal Antigen Typing System (MATS), has been used to predict vaccine coverage of MenB-4C. However, MATS only predicts coverage of antigens specific to MenB-4C and is not useful for assessing coverage of other vaccines with different antigen compositions. Specifically, MATS measures antigen expression rather than bactericidal activity and is reported as a relative potency compared with a reference strain for each antigen. If the relative potency for any one of the component antigens is commensurate with bactericidal activity for MenB-4C immune sera (ie, achieves a positive bactericidal threshold), the strain is considered susceptible to killing. However, because sera from vaccinated individuals are not used in MATS, the assay is unable to predict the proportion of a population achieving hSBA titers 21:4 (ie, the correlate of protection) in response to immunization.

Of note, limitations in performing hSBAs exist. For example, hSBAs are labor intensive and can require large quantities of sera and assay-compatible complement, particularly when larger numbers of strains and/or sera are to be assessed. In addition, interlaboratory differences in the performance of the assay reagents and strains used in hSBAs limit comparison of responses and assessments of breadth of coverage between vaccines. A known limitation of PPV analysis is the dependence of the magnitude of the response on prevalence (ie, in this setting, the proportion of subjects achieving hSBA ≥LLOQ for the additional strains). However, it is notable in this analysis that although there was a range of postvaccination responses to the additional strains (at 1 month postdose 2 and postdose 3), PPVs were uniformly high.

Taken together, the immunogenicity data obtained from the 10 additional MenB hSBA test strains support the response data obtained from the 4 primary MenB hSBA test strains and confirm the broad coverage of MenB isolates conferred by MenB-FHbp. This is the first work that has applied a rigorous assessment of a MenB vaccine's elicited immune response using the epidemiology of MenB strains with regard to the vaccine antigen sequence and expression, in conjunction with the recognized surrogate of protection (hSBA), and, using this knowledge, led to vaccine licensure.

### Methods

### Quantitation of FHbp Surface Expression

For all strains, FHbp surface expression was quantified by the MEASURE assay, a flow cytometric assay using monoclonal antibody (MN86-994-11) recognition of a conserved FHbp epitope common to both FHbp subfamilies. Details of the MEASURE assay have been described previously. The cutoff level adopted for each FHbp variant group was the observed median mean fluorescence intensity plus 1 standard deviation, using the precision estimate of 25.2% relative standard deviation.

### Immunogenicity Analysis

Each of the 10 additional MenB test strains were used in hSBAs to test sera from subjects participating in 2 pivotal phase 3 studies of MenB-FHbp. A total of 900 subjects from each study were to be divided into 3 subsets (n=300 each); the 10 additional test strains were allocated across these subsets so that 2 subsets each included 3 test strains and 1 subset included 4 test strains. The subsets included samples from 300 subjects to ensure that 150 evaluable hSBA results from each study would be obtained. Immune responses measured by hSBA using phase 3 clinical study sera were based on the assay LLOQ, which was an hSBA titer equal to 1:8 or 1:16 depending on the strain.

### Positive Predictive Value Analyses

The PPV for each primary/additional strain pair within an FHbp subfamily was defined as the proportion of subjects responding to the additional strain (hSBA titer LLOQ for the additional strain) among the total number of primary strain responders (hSBA titer LLOQ for the primary strain). PPV analyses assessed whether observed hSBA responses to the 4 primary strains predicted immune responses to additional strains expressing FHbps from the same subfamily.

### Example 20: Pentavalent Meningococcal (MenABCWY) Vaccine is Safe and Well Tolerated with Immunogenicity Noninferior to Coadministered MenB-FHbp and MenACWY-CRM in a Phase 2 Study of Healthy Adolescents and Young Adults

Background: Meningococcal serogroup A, B, C, W and Y cause nearly all meningococcal disease globally. Vaccination is complicated by different dosing recommendations for serogroup B (MenB) and quadrivalent (MenACWY) vaccines, which could be solved with a single pentavalent vaccine. This study in adolescents and young adults evaluated a new pentavalent MenABCWY vaccine that combines 2 licensed vaccines, MenB-FHbp (TRUMENBA®; bivalent rLP2086) and MenACWY-TT (NIMENRIX®), into a single vaccine.

Methods: In this ongoing, randomized, controlled, observer-blinded, multicenter study (NCTO3135834), MenB vaccine-naive and MenACWY-naive or -experienced healthy 10-25-year-olds were randomized 1:2 to MenABCWY (Month 0,6) or MenB-FHbp (Month 0,6) and MenACWY-CRM (Month 0). Immune responses were measured by serum bactericidal activity assays with human complement (hSBA) against serogroup A, C, W and Y strains and 4 diverse, vaccine-heterologous MenB strains. Endpoints included percentages of subjects achieving 4-fold rises in titers from baseline. Noninferiority of immune responses were assessed a priori at the 10% margin (95% CI lower limit>−10%). Safety was assessed.

Results: Following dose 2, high percentages of MenABCWY (n=543) and MenB-FHbp (n=1057) recipients achieved 4-fold rises against each of the 4 MenB strains (75.8-94.7% vs 67.4-95.0%) and titers at least the lower limit of quantification against all 4 strains combined (79.9% vs 74.3%; FIG. 1A). MenABCWY was noninferior to MenB-FHbp for all 5 endpoints. MenABCWY was also noninferior to a single MenACWY-CRM dose with 75.5-96.9% and 93.0-97.4% of MenABCWY recipients after dose 1 or 2, respectively, achieving 4-fold rises against serogroup A, C, W and Y depending on prior MenACWY experience (FIG. 1B). Local reactions and systemic events after MenABCWY or MenB-FHbp were similarly frequent, mostly mild/moderate in severity (FIG. 2), and unaffected by MenACWY experience.

6.2.2.3 Immunogenicity Results from MenABCWY Clinical Development Program

(Study B1971057) This portion of the study was the Phase 2 component of the overall study. For the MenB component of MenABCWY, the proportions of MenABCWY and bivalent rLP2086+MenACWY-CRM recipients achieving a 4-fold rise from baseline in hSBA titer at 1 month after Vaccination 2 were 75.8% and 73.8%, respectively, for PMB80 (A22); 94.7% and 95.0%, respectively, for PMB2001 (A56); 76.1% and 67.4%, respectively, for PMB2948 (B24); and 91.7% and 86.4%, respectively, for PMB2707 (B44). The proportions of MenABCWY and bivalent rLP2086+MenACWY-CRM recipients achieving a composite response (hSBA titers LLOQ for all 4 MenB test strains) at 1 month after Vaccination 2 were 79.9% and 74.3%, respectively.

Conclusion: These results indicate that MenABCWY, whether given as a single dose or as a 2-dose series separated by 6 months, provides a high degree of protective immunity against MenB after 2 doses and MenACWY after 1 or 2 doses similar to that achieved when administering bivalent rLP2086 (0, 6-month) and MenACWY-CRM (0-month) separately, regardless of prior ACWY experience. MenABCWY 4-fold immune responses from baseline were robust and noninferior to MenB-FHbp and MenACWY-CRM administered separately. Vaccination on a 0-, 6-month schedule was safe and well tolerated. The favorable benefit-risk profile supports further MenABCWY development as a simplified alternative to current meningococcal vaccination practices.

Data generated from study B1971057 (MenABCWY[FIH]) demonstrated responses to MenABCWY to be noninferior to TRUMENBA (meningococcal group B bivalent recombinant lipoprotein 2086 Vaccine) (MenB evaluation) and MENVEO (meningococcal (groups A, C, Y, and W-135) oligosaccharide CRM197 conjugate vaccine) (MenACWYCRM evaluation) when administered to healthy individuals 10 to 25 years of age (see Section 5.2). There was no immune interference between the component parts of MenABCWY observed in this FIH study.

### Example 21: Effect of Shipping Stresses on Suspension Vaccines

This example illustrates the effect of shipping stresses (shock/drop, vibration, low-pressure/high altitude, and temperature) on the suspension vaccine. Re-dispersion of the vaccine suspension is an important consideration. Understanding the factors that can affect re-dispersion times to minimize re-dispersion time for end-users is the key product development goal. We present the systematic way of assessing the parameters that affect the re-dispersion time for a suspension vaccine and associated control strategy.

Suspension vaccines are thermodynamically unstable systems that constitute significant challenges in maintaining physical properties during storage and shipping in comparison to liquid or lyophilized powder. Although shipping stresses could impact product quality as well as the physical properties of a vaccine product, this example focuses on the impact of shipping stresses on the physical properties of suspension vaccines. Implications for product quality is out of scope since product quality is molecule dependent and can be evaluated by product-specific analytical and biological methods.

Aluminum-containing adjuvants have been included in vaccines to enhance or modify the immune response to antigens for over seven decades. Aluminum-containing vaccine is a suspension in which the internal phase consists of insoluble aluminum-containing salts (aluminum phosphate or aluminum hydroxide), and the external phase is a liquid vehicle. The addition of insoluble adjuvants to a vaccine drug product results in a suspension where the dispersed phase tends to settle with time. Ideally, a suspension should be uniform, and any sedimentation which occurs during storage can be easily re-dispersed on agitation. Compare a pre-filled syringe containing vaccine suspension before and after re-dispersion. Often enough, the re-dispersion issue is prevalent after the suspension is filled into pre-filled syringes and kept for long term storage. Countless research has focused on the addition of controlled flocculating agents or polymer additives to form a large loose and easily dispersible sediments4. Several commercial aluminum-based vaccines clearly instruct in their package insert to “shake vigorously immediately before use.” On Apr. 26, 2010, WHO recommended recall and destruction of all lots of SHAN 5 vaccine as a precautionary measure following incidents of white sediment sticking to SHAN 5 vaccine vials that were difficult or impossible to re-suspend5. Therefore, re-dispersion of vaccine suspension should be evaluated thoroughly to understand the factors affecting the re-dispersion time, and if needed, mitigation countermeasures can be placed.

INTERPLAY BETWEEN PARTICLE SIZE, CHARGE AND SETTLING RATE. It is essential to understand the factors that affect suspension stability as we seek to understand the cause for increased redispersion time. In this section, background on considerations for suspension stability is presented along with a case study of two suspensions to understand the interplay between particle size, charge, and settling of the suspension.

In an ideal suspension system, the dispersed phase is suspended for a prolonged time, and if settling of the sparse system happens, the dispersed system can be easily resuspended or dispersed. Suspension stability can be achieved through thermodynamic or kinetic means. Thermodynamic stability approaches include imparting charge on the suspension particle surface, thereby inducing steric hindrance due to particle-particle repulsion leading to suspension stabilization. On the other hand, a kinetic approach includes increasing viscosity of the suspension results in reduced settling of suspension and, hence providing the stability. Particle size plays an essential role in this interplay of thermodynamic and kinetic stability. For suspension systems which have particle size more than sub-micron, density differences between the clear phase and dispersed phase are significant factors and can lead to settling of the dispersed phase due to gravity. It is reported that particle size may affect the flocculation or coagulation of a suspension system. PEG, for example, is added to a lot of injectable solid/liquid suspensions to allow the solid active ingredient particles to form bigger flocculated sediment, thus making the sediment easier to resuspend. Whereas super-micron particles are ideally suited to slow down the dissolution kinetics, they are much more affected by gravity effects, which may lead to rather compact and hardly re-suspendable sediment.

Sedimentation or settling is a result of collective interactions in concentrated suspensions. Collective interactions include hydrodynamic and particle-particle interactions (determined through charge-charge interactions). Hydrodynamic effects account for the retardation of the backflow, which is the reverse flow of fluid to compensate for the movement of settling particles. Particle-particle interactions are due to the attractive self-depletion and repulsive structural forces in concentrated dispersions.

Two suspension systems are illustrated to describe the interplay between charge, settling rate, and particle size. Vaccine suspension drug product in the study is designated as suspension 1 and 2 for this illustration. Suspension 1 and suspension 2 depict comparable particle size (approximately 15 μm). These suspensions behave vastly differently when settling rates are compared. Suspension 1 settles significantly faster with settling rate of approximately 0.04 abs/min while suspension 2 takes weeks even to start settling. This difference is stark despite having comparable particle size distribution. As discussed earlier, an understanding charge on the particle surface becomes essential. Zeta potential for suspension 1 is approximately −5 mV, while suspension 2 has a zeta potential of −45 mV. Since suspension 1 has a little charge on the particle surface, thermodynamic suspension instability promotes particle-particle attraction and eventually settling of the dispersed phase.

On the other hand, high charge for suspension 2 system makes the systemic thermodynamically stable and particle-particle repulsion leads to significantly slow settling of the dispersed phase. Based on this, suspension 2 imparts maximum thermodynamic stability and is desirable. Although the biggest question is whether the thermodynamically stable suspension is easier to disperse after the dispersed system settles or vice versa, how does this translate into ease of suspension in syringes for vaccine suspensions?Our data with these two suspensions suggests that although suspension 1 is thermodynamically unstable due to lack of charge results in particle-particle interaction and possibly the formation of bigger flocculated sediment resulting in faster sedimentation and hence making the dispersed phase system easily resuspendable. On the other hand, suspension 2 takes a significantly longer time to settle due to charge-charge polarity, although once settled, the finer particles pack together to form significantly harder to resuspend settled dispersed phase.

Based on these observations and background, two key questions were tested in our studies to understand the increase in redispersion time for suspension 1 vaccine: Can shipping stress alter thermodynamic suspension stability resulting in increased redispersion time?What is the impact of individual shipping stress components during shipping, and if there is a correlation to increased redispersion time?

### Effect of Shipping Stress on Thermodynamic Suspension Stability

In another study, vaccine drug product suspension was subjected to simulated shipping stresses including shipping temperature, shock/drop and vibration. Thermodynamic suspension stability was determined through measurement of charge (zeta potential), particle size distribution, and settling rate before and after shipping.

Drug product suspension exhibited zeta potential values around −5 mV, suggesting that there is a little charge on suspension particles. This thermodynamic instability of the suspension promotes particle-particle attraction and eventually settling of the dispersed phase. Our data suggest that there were no apparent changes in zeta potential value, particle size distribution, and measured settling rate before and after the simulated shipping of syringes. These results suggest that shipping stress does not alter the thermodynamic suspension stability for drug product suspension. Since these properties are not changed after shipping, it can be concluded that thermodynamic stability is not the driving factor for observed increased redispersion time and necessitates the need to evaluate the effect of shipping stresses and understand if any specific physical mechanism is contributing to the observed redispersion time.

### Impact of Individual Shipping Stresses on Redispersion Time

When a package is shipped, the product will distinctly experience various shipping stresses. These include shock/drop stress when the package is dropped from various heights, vibrations during either truck or aircraft transport, although the amplitude of vibration frequency is different between aircraft and truck. The impact of individual shipping stress on redispersion time was assessed when drug product syringes were subjected to simulated shipping¹¹. The study design also included the assessment of the impact of shipping stress as a function of syringe orientation. Syringes were kept in three distinct orientations: tip cap down, tip cap up, and tip cap horizontal. The study aims to decipher individual stresses and understand the impact.

It was observed that shock/drop stress reduced re-dispersion time for tip cap down orientation, and there was little effect of shock/drop on the re-dispersion in tip cap horizontal and tip cap up orientations.

On the other hand, aircraft vibration increased re-dispersion time for tip cap down orientation. This was an interesting finding and can be related to the vibration frequencies encountered during aircraft transportation. Vibration frequency when syringes are in tip cap down orientation can further facilitate the particles of the suspension to go down the syringe bore. There was little effect of aircraft vibration on the syringes arranged tip cap up and horizontal. Similar to aircraft vibration, truck vibration increased re-dispersion time in tip cap down orientation. These findings are similar to aircraft vibration, although the extent of increase in redispersion time was more compared to aircraft vibration. Additionally, there was little effect of truck vibration on the syringes arranged tip cap up and horizontally consistent with aircraft vibration.

Also, when the syringes were assessed for redispersion time for combination stress (shock/drop, aircraft vibration, truck vibrational shock/drop), the data was confounding, suggesting that in actual shipping, most likely one or more modes of shipping stress can dominate the results-producing utterly different outcome.

It was interesting to note that vibration frequency intensity is an important factor based on the differences seen in aircraft and truck vibration. The increase in re-dispersion time when the syringe is placed in tip cap down orientation can be because the downward movement of suspension particles originated from gravity and aggravated by vibration facilitates tight packing of larger particles with the smaller particles filling the void spaces. Particles at the bottom, especially the bore of the syringe, are gradually pressed together by the weight of the ones above.

When a drug product is shipped in tip cap up orientation, gravity effect still exists, but sedimentation happens in the broader surface of the plunger instead of the narrow bore. The packing of the mass is not apparent. When drug product is shipped in tip cap horizontal orientation, the lateral motion resulted from Brownian movement and convection current from vibration overcome gravity. Even when sedimentation does happen, it will occur on the large surface of the syringe wall with little chance of packing the particles. Therefore, re-suspension is not an issue for both tip cap up and tip cap horizontal orientations with tip cap horizontal orientation yielding slightly better results presumably due to larger surface area. When shipped, shipping vibration is the dominant force that results in the end state, where sedimentation is packed. Depending on the orientation of the syringe and different surface areas available, re-dispersion time corresponds to these open surface areas.

### Mitigation Strategy to Reduce High Redispersion Time

Based on the simulated shipping study data, it is clear that either tip cap horizontal or tip cap up orientation can mitigate the higher re-dispersion times for the vaccine suspension.

### Impact of Shipping Temperature on Suspension Vaccines

In addition to re-dispersion, product quality of vaccine suspensions could also be affected by temperature during shipping. After freezing, the bond between adjuvant and antigen could be broken. Separated adjuvant tends to form agglomerate that gets bigger in particle size and weight, then gradually settles to the bottom of the container. The size of the agglomerate may increase after repeated freezing and thawing cycles. The formation of agglomerate impacts both re-dispersion, physical and chemical properties of the product. WHO Guidelines on the International Packaging and Shipping of Vaccines have attached a shake test protocol to determine whether adsorbed vaccines have been affected by freezing. The guidelines also specify the shake test on a random sample of vaccines if there is an indication that temperatures have dropped below zero during transportation. Higher temperatures, on the other hand, could also impact product quality of vaccine suspension, resulting in particle formation and chemical property changes or both. Therefore, WHO specifies +8° C. as the maximum temperature allowed inside the insulated Class A packaging during international transport, for at least 48 hours. The maximum temperature allowed for Class B and C packaging is +30° C. The following clauses describe additional embodiments of the invention:
C1. A composition comprising (a) a first polypeptide derived from a Neisseria meningitidis factor H binding protein (fHBP); (b) a second polypeptide derived from a Neisseria meningitidis factor H binding protein (fHBP); (c) a Neisseria meningitidis serogroup A capsular saccharide conjugate; (d) a Neisseria meningitidis serogroup C capsular saccharide conjugate; (e) a Neisseria meningitidis serogroup W capsular saccharide conjugate; and (f) a Neisseria meningitidis serogroup Y capsular saccharide conjugate; wherein the composition elicits an immune response to any one of the N. meningitidis serogroups A, C, W-135 and Y capsular polysaccharides, wherein said serum bactericidal antibody response is higher than that elicited by a licensed vaccine against the N. meningitidis serogroup.
C2. The method according to clause C1, wherein the polypeptide comprises an amino acid sequence having at least 70% identity to any one amino acid sequence selected from SEQ ID NO: 1 to SEQ ID NO: 62.
C3. The method according to clause C1, wherein the composition comprises (a) a first polypeptide comprising the amino acid sequence set forth in SEQ ID NO: 1; (b) a second polypeptide comprising the amino acid sequence set forth in SEQ ID NO: 2; (c) a Neisseria meningitidis serogroup A capsular saccharide conjugated to an adipic acid dihydrazide (ADH) linker by 1-cyano-4-dimethylamino pyridinium tetrafluoroborate, wherein the linker is conjugated to tetanus toxoid by carbodiimide chemistry; (d) a Neisseria meningitidis serogroup C capsular saccharide conjugated to an ADH linker by 1-cyano-4-dimethylamino pyridinium tetrafluoroborate, wherein the linker is conjugated to tetanus toxoid by carbodiimide chemistry; (e) a Neisseria meningitidis serogroup W capsular saccharide directly conjugated to tetanus toxoid by 1-cyano-4-dimethylamino pyridinium tetrafluoroborate, in the absence of a linker; and (f) a Neisseria meningitidis serogroup Y capsular saccharide directly conjugated to tetanus toxoid by 1-cyano-4-dimethylamino pyridinium tetrafluoroborate, in the absence of a linker.
C4. The method according to any one of clause C1 to clause C3, wherein the composition elicits an immune response to any one of N. meningitidis serogroups A, C, W-135 and Y, wherein said serum bactericidal antibody response is higher than that elicited by a licensed N. meningitidis serogroups A, C, W-135 and Y meningococcal capsular polysaccharide vaccine.
C5. The method according to any one of clause C1 to clause C3, wherein the composition elicits an immune response to N. meningitidis serogroup A, wherein said serum bactericidal antibody response is higher than that elicited by a licensed N. meningitidis serogroups A, C, W-135 and Y meningococcal capsular polysaccharide vaccine.
C6. The method according to any one of clause C1 to clause C3, wherein the composition elicits an immune response to N. meningitidis serogroup C, wherein said serum bactericidal antibody response is higher than that elicited by a licensed N. meningitidis serogroups A, C, W-135 and Y meningococcal capsular polysaccharide vaccine.
C7. The method according to any one of clause C1 to clause C3, wherein the composition elicits an immune response to N. meningitidis serogroup W, wherein said serum bactericidal antibody response is higher than that elicited by a licensed N. meningitidis serogroups A, C, W-135 and Y meningococcal capsular polysaccharide vaccine.
C8. The method according to any one of clause C1 to clause C3, wherein the composition elicits an immune response to N. meningitidis serogroup Y, wherein said serum bactericidal antibody response is higher than that elicited by a licensed N. meningitidis serogroups A, C, W-135 and Y meningococcal capsular polysaccharide vaccine.
C9. The method according to any one of clause C1 to clause C3, wherein the composition elicits an immune response to each of the N. meningitidis serogroups A, C, W-135 and Y, wherein said serum bactericidal antibody response is higher than that elicited by a licensed N. meningitidis serogroups A, C, W-135 and Y meningococcal capsular polysaccharide vaccine.
C10. The method according to any one of clause C1 to clause C3, wherein the composition elicits an immune response to N. meningitidis serogroup B, wherein said serum bactericidal antibody response is higher than that elicited by a licensed meningococcal serogroup B factor H binding vaccine.
C11. The method according to any one of clause C1 to clause C3, wherein the composition elicits an immune response to each of the N. meningitidis serogroups A, C, W-135 and Y, wherein said serum bactericidal antibody response to each of the N. meningitidis serogroups A, C, W-135 and Y capsular polysaccharides is higher than that elicited by a licensed N. meningitidis serogroups A, C, W-135 and Y meningococcal capsular polysaccharide vaccine; and the composition elicits an immune response to N. meningitidis serogroup B, wherein said serum bactericidal antibody response is higher than that elicited by a licensed meningococcal serogroup B factor H binding vaccine; wherein the licensed N. meningitidis serogroups A, C, W-135 and Y meningococcal capsular polysaccharide vaccine and the licensed meningococcal serogroup B factor H binding vaccine are administered sequentially and are not in a combined dose.
C12. The method according to any one of clause C1 to clause C3, wherein the composition comprises an adjuvant.
C13. The method according to any one of clause C1 to clause C3, wherein the composition comprises an aluminum adjuvant.
C14. The method according to any one of clause C1 to clause C3, wherein the composition comprises aluminum hydroxide.
C15. The method according to any one of clause C1 to clause C3, wherein the composition comprises aluminum phosphate.
C16. The method according to any one of clause C1 to clause C3, wherein the composition comprises comprising aluminum.
C17. The method according to any one of clause C1 to clause C3, wherein at least 90% of the first polypeptide is bound to aluminum in the composition.
C18. The method according to any one of clause C1 to clause C3, wherein at least 90% of the second polypeptide is bound to aluminum in the composition.
C19. The method according to any one of clause C1 to clause C3, wherein the composition is formulated as a sterile liquid.
C20. The method according to any one of clause C1 to clause C3, wherein the composition comprises a pharmaceutically acceptable preservative.
C21. The method according to any one of clause C1 to clause C3, wherein the composition comprises polysorbate-80.
C22. The method according to any one of clause C1 to clause C3, wherein the composition comprises Tris-HCl; sodium chloride; sucrose; histidine; polysorbate 80; and aluminum phosphate.
C23. The method according to any one of clause C1 to clause C3, wherein the composition comprises about 120 μg/ml of the first polypeptide; about 120 μg/ml of the second polypeptide; about 0.5 mg/ml aluminum as aluminum phosphate; about 0.02 mg polysorbate-80; about 10 mM histidine; and about 150 mM sodium chloride.
C24. The method according to any one of clause C1 to clause C3, wherein the composition comprises about 60 μg of the first polypeptide; about 60 μg of the second polypeptide; about 5 μg of the MenA capsular saccharide conjugated to about 7.5 μg TT; about 5 μg of the MenC capsular saccharide conjugated to about 7.5 μg TT; about 5 μg of the MenW capsular saccharide conjugated to about 3.75 μg TT; about 5 μg of the MenY capsular saccharide conjugated to about 3.25 μg TT; about 97 μg Tris-HCl, pH 6.8±0.3; 4.69-4.71 mg of sodium chloride; about 28 mg of sucrose; about 0.78 mg of L-Histidine; about 0.02 mg polysorbate-80; about 0.25 mg aluminum; and further comprising 0.5 mL water, per dose.
C25. The method according to any one of clause C1 to clause C3, wherein the immune response comprises a serum bactericidal antibody.
C26. The method according to any one of clause C1 to clause C3, wherein the composition is capable of eliciting a booster immune response to at least one of the N. meningitidis serogroups A, C, W-135 and Y.
C27. The method according to any one of clause C1 to clause C3, wherein the composition is capable of eliciting a booster immune response to N. meningitidis serogroup B.
C28. The method according to any one of clause C1 to clause C3, wherein the immune response is elicited in a human 10 to 26 years old.
C29. The method according to any one of clause C1 to clause C3, wherein the immune response is elicited in a human aged 12 to <18 Months or 18 to <24 Months.
C30. The method according to any one of clause C1 to clause C3, wherein the immune response is elicited in a human aged 18 to <24 Months.
C31. The method according to any one of clause C1 to clause C3, wherein the immune response is elicited in a human aged ≥24 Months to <10 Years.
C32. The method according to any one of clause C1 to clause C3, wherein the immune response is elicited in a human that is seronegative against N. meningitidis serogroups A, C, W-135 and Y.
C33. The method according to any one of clause C1 to clause C3, wherein the immune response is elicited in a human that is seropositive against N. meningitidis serogroups A, C, W-135 and Y.
C34. The method according to any one of clause C1 to clause C3, wherein the composition is administered to the human in at least two doses, wherein the second dose is about 6 months after the first dose.
C35. The method according to clause C34, wherein the human is at least 10 years of age and at most 17 years of age.
C36. The method according to clause C35, wherein a third dose of the composition is administered to the human, wherein the human is at least 16 years of age.
C37. The method according to any one of clause C1 to clause C3, wherein the composition is administered to the human in at most two doses, wherein the second dose is about 6 months after the first dose.
C38. The method according to any one of clause C1 to clause C3, wherein the composition elicits an immune response against A22.
C39. The method according to any one of clause C1 to clause C3, wherein the composition elicits an immune response against A56.
C40. The method according to any one of clause C1 to clause C3, wherein the composition elicits an immune response against B24.
C41. The method according to any one of clause C1 to clause C3, wherein the composition elicits an immune response against B44.
C42. The method according to any one of clause C1 to clause C3, wherein the composition comprises about 60 μg of the first polypeptide; about 60 μg of the second polypeptide; about 5 μg of the MenA capsular saccharide conjugated to about 7.5 μg TT; about 5 μg of the MenC capsular saccharide conjugated to about 7.5 μg TT; about 5 μg of the MenW capsular saccharide conjugated to about 3.75 μg TT; about 5 μg of the MenY capsular saccharide conjugated to about 3.25 μg TT; about 97 μg Tris-HCl, pH 6.8±0.3; 4.69-4.71 mg of sodium chloride; about 28 mg of sucrose; about 0.78 mg of L-Histidine; about 0.02 mg polysorbate-80; about 0.25 mg aluminum; and further comprising 0.5 mL water, per dose.
C43. A composition comprising (a) a first polypeptide derived from a Neisseria meningitidis factor H binding protein (fHBP); (b) a second polypeptide derived from a Neisseria meningitidis factor H binding protein (fHBP); (c) a Neisseria meningitidis serogroup A capsular saccharide conjugate; (d) a Neisseria meningitidis serogroup C capsular saccharide conjugate; (e) a Neisseria meningitidis serogroup W capsular saccharide conjugate; and (f) a Neisseria meningitidis serogroup Y capsular saccharide conjugate; wherein the composition elicits an immune response to at least one of N. meningitidis serogroups A, C, W-135 and Y, wherein said serum bactericidal antibody response is higher than that elicited by a licensed vaccine against the N. meningitidis serogroup.
C44. The composition according to clause C43, wherein the polypeptide comprises an amino acid sequence having at least 70% identity to any one amino acid sequence selected from SEQ ID NO: 1 to SEQ ID NO: 62.
C45. A composition comprising (a) a first polypeptide derived from a Neisseria meningitidis factor H binding protein (fHBP); (b) a second polypeptide derived from a Neisseria meningitidis factor H binding protein (fHBP); (c) a Neisseria meningitidis serogroup A capsular saccharide conjugated to an adipic acid dihydrazide (ADH) linker by 1-cyano-4-dimethylamino pyridinium tetrafluoroborate, wherein the linker is conjugated to tetanus toxoid by carbodiimide chemistry; (d) a Neisseria meningitidis serogroup C capsular saccharide conjugated to an ADH linker by 1-cyano-4-dimethylamino pyridinium tetrafluoroborate, wherein the linker is conjugated to tetanus toxoid by carbodiimide chemistry; (e) a Neisseria meningitidis serogroup W capsular saccharide directly conjugated to tetanus toxoid by 1-cyano-4-dimethylamino pyridinium tetrafluoroborate, in the absence of a linker; and (f) a Neisseria meningitidis serogroup Y capsular saccharide directly conjugated to tetanus toxoid by 1-cyano-4-dimethylamino pyridinium tetrafluoroborate, in the absence of a linker; wherein the composition elicits an immune response to each of the N. meningitidis serogroups A, C, W-135 and Y capsular polysaccharides, wherein said serum bactericidal antibody response is higher than that elicited by a licensed N. meningitidis serogroups A, C, W-135 and Y meningococcal capsular polysaccharide vaccine.
C46. A method for inducing an immune response against a Neisseria meningitidis serogroup B subfamily A strain and against a Neisseria meningitidis serogroup B subfamily B strain in human, comprising administering to the human an effective amount of the composition according to any one of clause C43 to clause C45.
C47. A method for inducing an immune response against a Neisseria meningitidis serogroup A, a Neisseria meningitidis serogroup C, a Neisseria meningitidis serogroup W, and/or a Neisseria meningitidis serogroup Y strain in a human, comprising administering to the human an effective amount of the composition according to any one of clause C43 to clause C45.
C48. A method for inducing an immune response against a Neisseria meningitidis serogroup A, Neisseria meningitidis serogroup B, a Neisseria meningitidis serogroup C, a Neisseria meningitidis serogroup W, and/or a Neisseria meningitidis serogroup Y strain in a human, comprising administering to the human an effective amount of the composition according to any one of clause C43 to clause C45.
C49. A method for inducing an immune response against a Neisseria meningitidis serogroup A, Neisseria meningitidis serogroup B, a Neisseria meningitidis serogroup C, a Neisseria meningitidis serogroup W, a Neisseria meningitidis serogroup Y strain, and/or a Neisseria meningitidis serogroup X strain in a human, comprising administering to the human an effective amount of the composition according to any one of clause C43 to clause C45.
C50. The method according to any one of clauses C46 to C49, wherein the patient has not previously received a multivalent meningococcal capsular saccharide-carrier protein conjugate vaccine prior to the first administration of the composition according to any one of clause C43 to clause C45.
C51. The method according to any one of clauses C46 to C49, wherein the patient previously received a multivalent meningococcal capsular saccharide-carrier protein conjugate vaccine prior to the first administration of the composition according to any one of clause C1 and clause C10.
C52. Use of an effective amount of a composition for inducing an immune response against Neisseria meningitidis serogroup B in a human, wherein said composition comprises a) a first lipidated polypeptide comprising the amino acid sequence set forth in SEQ ID NO: 1, and b) a second lipidated polypeptide comprising the amino acid sequence set forth in SEQ ID NO: 2, wherein the composition induces an immune response against at least one N. meningitidis serogroup B strain expressing a polypeptide selected from the group consisting of A02, A28, A42, A63, A76, B05, B07, B08, B13, B52 and B107.
C53. The use according to clause C52, wherein the immune response induced is bactericidal.
C54. The use according to clause C52, wherein the composition further comprises polysorbate-80.
C55. The use according to any one of clauses C52 to C54, wherein the composition further comprises aluminum.
C56. The use according to any one of clauses C52 to C55, wherein the composition further comprises histidine buffer.
C57. The use according to any one of clauses C52 to C56, wherein the composition further comprises sodium chloride.
C58. The use according to any one of clauses C52 to C57, wherein the composition comprises about 120 μg/ml of the first polypeptide; about 120 μg/ml of the second polypeptide; about 2.8 molar ratio of polysorbate-80; about 0.5 mg/ml aluminum; about 10 mM histidine; and about 150 mM sodium chloride.
C59. The use according to any one of clauses C52 to C58, wherein the composition comprises about 60 μg of the first polypeptide; about 60 μg of the second polypeptide; about 18 μg polysorbate-80; about 250 μg aluminum; about 780 μg histidine; and about 4380 μg sodium chloride.
C60. The use according to any one of clauses C52 to C59, wherein the composition further comprises at least one additional immunogenic composition comprising a mixture of four distinct and separately made protein-capsular polysaccharide conjugates, wherein the first conjugate comprises N. meningitidis capsular polysaccharide of serogroup W conjugated to a carrier protein, the second conjugate comprises N. meningitidis capsular polysaccharide of serogroup Y conjugated to a carrier protein, the third conjugate comprises N. meningitidis capsular polysaccharide of serogroup A conjugated to a carrier protein, and the fourth conjugate comprises N. meningitidis capsular polysaccharide of serogroup C conjugated to a carrier protein, wherein the carrier protein is selected from the group consisting of diphtheria toxoid, CRM₁₉₇, and tetanus toxoid.
C61. The use according to clause C60, wherein the carrier protein is diphtheria toxoid.
C62. The use according to clause C60, wherein the carrier protein is tetanus toxoid.
C63. The use according to clause C60, wherein the at least one additional immunogenic composition is a liquid composition.
C64. The use according to clause C60, wherein the at least one additional immunogenic composition is not lyophilized.
C65. The use according to any one of clauses C60 to C64, wherein the composition induces an immune response against at least one Neisseria meningitidis serogroup A strain.
C66. The use according to any one of clauses C60 to C64, wherein the composition induces an immune response against at least one Neisseria meningitidis serogroup C strain.
C67. The use according to any one of clauses C60 to C64, wherein the composition induces an immune response against at least one Neisseria meningitidis serogroup W strain.
C68. The use according to any one of clauses C60 to C64, wherein the composition induces an immune response against at least one Neisseria meningitidis serogroup Y strain.
C69. The use according to any one of clauses C60 to C64, wherein the composition induces an immune response against at least one of a Neisseria meningitidis serogroup A strain, a Neisseria meningitidis serogroup C strain, a Neisseria meningitidis serogroup Y strain, a Neisseria meningitidis serogroup W strain, and any combination thereof.
C70. The use according to any one of clauses C52 to C69, wherein the effective amount of the composition comprises one dose.
C71. The use according to any one of clauses C52 to C70, wherein the effective amount of the composition comprises two doses.
C72. The use according to any one of clauses C52 to C70, wherein the effective amount of the composition further comprises a booster dose.
C73. The use according to any one of clauses C52 to C70, wherein the effective amount of the composition comprises at most two doses.
C74. The use according to any one of clauses C52 to C70, wherein the effective amount of the composition comprises at most three doses.
C75. The use according to clause C52, wherein the composition does not comprise a hybrid protein.
C76. The use according to clause C52, wherein the composition does not comprise a fusion protein.
C77. The use according to clause C52, wherein the composition is not lyophilized.
C78. The use according to clause C52, wherein the composition does not comprise formaldehyde.
C79. The use according to clause C60, wherein the composition does not comprise diphtheria toxoid or CRM.
C80. The use according to clause C60, wherein the Neisseria meningitidis serogroup A (MenA) capsular saccharide is conjugated to an adipic acid dihydrazide (ADH) linker by 1-cyano-4-dimethylamino pyridinium tetrafluoroborate chemistry, wherein the linker is conjugated to tetanus toxoid carrier protein (TT) by carbodiimide chemistry (MenA_{AH}-TT conjugate).
C81. The use according to clause C60, wherein the Neisseria meningitidis serogroup C (MenC) capsular saccharide is conjugated to an ADH linker by 1-cyano-4-dimethylamino pyridinium tetrafluoroborate chemistry, wherein the linker is conjugated to tetanus toxoid carrier protein (TT) by carbodiimide chemistry (MenC_{AH}-TT conjugate).
C82. The use according to clause C60, wherein the Neisseria meningitidis serogroup W (MenW) capsular saccharide is directly conjugated to tetanus toxoid carrier protein (TT) by 1-cyano-4-dimethylamino pyridinium tetrafluoroborate chemistry, in the absence of a linker (MenW-TT conjugate).
C83. The use according to clause C60, wherein the Neisseria meningitidis serogroup Y (MenY) capsular saccharide is directly conjugated to tetanus toxoid carrier protein (TT) by 1-cyano-4-dimethylamino pyridinium tetrafluoroborate chemistry, in the absence of a linker (MenY-TT conjugate).
C84. The use according to clause C60, wherein the Neisseria meningitidis serogroup A (MenA) capsular saccharide is conjugated to an adipic acid dihydrazide (ADH) linker by 1-cyano-4-dimethylamino pyridinium tetrafluoroborate chemistry, wherein the linker is conjugated to tetanus toxoid carrier protein (TT) by carbodiimide chemistry (MenA_{AH}-TT conjugate); the Neisseria meningitidis serogroup C (MenC) capsular saccharide is conjugated to an ADH linker by 1-cyano-4-dimethylamino pyridinium tetrafluoroborate chemistry, wherein the linker is conjugated to tetanus toxoid carrier protein (TT) by carbodiimide chemistry (MenC_{AH}-TT conjugate); the Neisseria meningitidis serogroup W (MenW) capsular saccharide is directly conjugated to tetanus toxoid carrier protein (TT) by 1-cyano-4-dimethylamino pyridinium tetrafluoroborate chemistry, in the absence of a linker (MenW-TT conjugate); and the Neisseria meningitidis serogroup Y (MenY) capsular saccharide is directly conjugated to tetanus toxoid carrier protein (TT) by 1-cyano-4-dimethylamino pyridinium tetrafluoroborate chemistry, in the absence of a linker (MenY-TT conjugate).
C85. The use according to clause C60, wherein the composition does not comprise a MenA capsular saccharide in the absence of an adipic acid dihydrazide (ADH) linker.
C86. The use according to any one of clauses C52 to C85, wherein the patient is aged 12 to <18 Months or 18 to <24 Months.
C87. The use according to any one of clauses C52 to C85, wherein the patient is aged 18 to <24 Months.
C88. The use according to any one of clauses C52 to C85, wherein the patient is aged ≥24 Months to <10 Years.
C89. The use according to any one of clauses C52 to C88, wherein the composition induces a bactericidal titer of serum immunoglobulin that is at least 2-fold higher in the human after receiving the first dose than a bactericidal titer of serum immunoglobulin in the human prior to receiving the first dose, when measured under identical conditions in a serum bactericidal assay using human complement.
C90. The use according to any one of clauses C52 to C89, wherein the composition induces a bactericidal titer of serum immunoglobulin that is at least 4-fold higher in the human after receiving the first dose than a bactericidal titer of serum immunoglobulin in the human prior to receiving the first dose, when measured under identical conditions in a serum bactericidal assay using human complement.
C91. The use according to any one of clauses C52 to C90, wherein the composition induces a bactericidal titer of serum immunoglobulin that is at least 8-fold higher in the human after receiving the first dose than a bactericidal titer of serum immunoglobulin in the human prior to receiving the first dose, when measured under identical conditions in a serum bactericidal assay using human complement.
C92. A method of inducing an immune response in a human, comprising administering to the human a composition comprising
   a) a first polypeptide comprising the amino acid sequence set forth in SEQ ID NO: 1; (b) a second polypeptide comprising the amino acid sequence set forth in SEQ ID NO: 2; (c) a Neisseria meningitidis serogroup A capsular saccharide conjugated to an adipic acid dihydrazide (ADH) linker by 1-cyano-4-dimethylamino pyridinium tetrafluoroborate, wherein the linker is conjugated to tetanus toxoid by carbodiimide chemistry; (d) a Neisseria meningitidis serogroup C capsular saccharide conjugated to an ADH linker by 1-cyano-4-dimethylamino pyridinium tetrafluoroborate, wherein the linker is conjugated to tetanus toxoid by carbodiimide chemistry; (e) a Neisseria meningitidis serogroup W capsular saccharide directly conjugated to tetanus toxoid by 1-cyano-4-dimethylamino pyridinium tetrafluoroborate, in the absence of a linker; and (f) a Neisseria meningitidis serogroup Y capsular saccharide directly conjugated to tetanus toxoid by 1-cyano-4-dimethylamino pyridinium tetrafluoroborate, in the absence of a linker,
wherein the composition induces an immune response to at least one of N. meningitidis serogroups A, C, W-135 and Y, wherein the immune response comprises a titer of serum bactericidal antibodies, and wherein the titer is higher than that induced by a licensed vaccine against N. meningitidis serogroups A, C, W-135 and Y meningococcal capsular polysaccharides.
C93. The method according to claim 1, wherein the licensed vaccine against N. meningitidis serogroups A, C, W-135 and Y meningococcal capsular polysaccharides is MENVEO.
C94. A method of inducing an immune response in a human, comprising administering to the human a composition comprising a) a first polypeptide comprising the amino acid sequence set forth in SEQ ID NO: 1; (b) a second polypeptide comprising the amino acid sequence set forth in SEQ ID NO: 2; (c) a Neisseria meningitidis serogroup A capsular saccharide conjugated to an adipic acid dihydrazide (ADH) linker by 1-cyano-4-dimethylamino pyridinium tetrafluoroborate, wherein the linker is conjugated to tetanus toxoid by carbodiimide chemistry; (d) a Neisseria meningitidis serogroup C capsular saccharide conjugated to an ADH linker by 1-cyano-4-dimethylamino pyridinium tetrafluoroborate, wherein the linker is conjugated to tetanus toxoid by carbodiimide chemistry; (e) a Neisseria meningitidis serogroup W capsular saccharide directly conjugated to tetanus toxoid by 1-cyano-4-dimethylamino pyridinium tetrafluoroborate, in the absence of a linker; and (f) a Neisseria meningitidis serogroup Y capsular saccharide directly conjugated to tetanus toxoid by 1-cyano-4-dimethylamino pyridinium tetrafluoroborate, in the absence of a linker, wherein the composition induces an immune response to N. meningitidis serogroup B, wherein the immune response comprises a titer of serum bactericidal antibodies that is higher than a titer of serum bactericidal antibodies induced by a licensed vaccine against N. meningitidis serogroup B.
C95. The method according to clause C94, wherein the licensed vaccine against N. meningitidis serogroup B is TRUMENBA.
C96. A method of inducing an immune response in a human, comprising administering to the human a composition comprising a) a first polypeptide comprising the amino acid sequence set forth in SEQ ID NO: 1; (b) a second polypeptide comprising the amino acid sequence set forth in SEQ ID NO: 2; (c) a Neisseria meningitidis serogroup A capsular saccharide conjugated to an adipic acid dihydrazide (ADH) linker by 1-cyano-4-dimethylamino pyridinium tetrafluoroborate, wherein the linker is conjugated to tetanus toxoid by carbodiimide chemistry; (d) a Neisseria meningitidis serogroup C capsular saccharide conjugated to an ADH linker by 1-cyano-4-dimethylamino pyridinium tetrafluoroborate, wherein the linker is conjugated to tetanus toxoid by carbodiimide chemistry; (e) a Neisseria meningitidis serogroup W capsular saccharide directly conjugated to tetanus toxoid by 1-cyano-4-dimethylamino pyridinium tetrafluoroborate, in the absence of a linker; and (f) a Neisseria meningitidis serogroup Y capsular saccharide directly conjugated to tetanus toxoid by 1-cyano-4-dimethylamino pyridinium tetrafluoroborate, in the absence of a linker, wherein the composition induces an immune response to at least one of N. meningitidis serogroups A, C, W-135 and Y and N. meningitidis serogroup B, wherein the immune response comprises a titer of serum bactericidal antibodies to each of N. meningitidis serogroups A, C, W-135 and Y that is higher than a titer of serum bactericidal antibodies induced by a licensed vaccine against N. meningitidis serogroups A, C, W-135 and Y capsular polysaccharide, and wherein the immune response comprises a titer of serum bactericidal antibodies to N. meningitidis serogroup B that is higher than a titer of serum bactericidal antibodies induced by a licensed vaccine against N. meningitidis serogroup B.
C97. The method according to clause C96, wherein the licensed vaccine against N. meningitidis serogroups A, C, W-135 and Y meningococcal capsular polysaccharides is MENVEO.
C98. The method according to any one of clauses C94 to C97, wherein the composition further comprises an adjuvant.
C99. The method according to any one of clauses C94 to C97, wherein the composition further comprises aluminum.
C100. The method according to clause C99, wherein the adjuvant comprises aluminum hydroxide.
C101. The method according to clause C99, wherein the adjuvant comprises aluminum phosphate.
C102. The method according to clause C94, wherein at least 90% of the first polypeptide is bound to aluminum in the composition.
C103. The method according to clause C94, wherein at least 90% of the second polypeptide is bound to aluminum in the composition.
C104. The method according to any one of clauses C94 to C97, wherein the composition is formulated as a sterile liquid.
C105. The method according to any one of clauses C94 to C97, wherein the composition further comprises polysorbate-80.
C106. The method according to any one of clauses C94 to C97, wherein the composition further comprises Tris-HCl; sodium chloride; sucrose; histidine; polysorbate 80; and aluminum phosphate.
C107. The method according to any one of clauses C94 to C97, wherein the composition comprises about 120 μg/ml of the first polypeptide; about 120 μg/ml of the second polypeptide; about 0.5 mg/ml aluminum as aluminum phosphate; about 0.02 mg polysorbate-80; about 10 mM histidine; and about 150 mM sodium chloride.
C108. The method according to any one of clauses C94 to C97, wherein the composition comprises about 60 μg of the first polypeptide; about 60 μg of the second polypeptide; about 5 μg of the MenA capsular saccharide conjugated to about 7.5 μg TT; about 5 μg of the MenC capsular saccharide conjugated to about 7.5 μg TT; about 5 μg of the MenW capsular saccharide conjugated to about 3.75 μg TT; about 5 μg of the MenY capsular saccharide conjugated to about 3.25 μg TT; about 97 μg Tris-HCl, pH 6.8±0.3; 4.69-4.71 mg of sodium chloride; about 28 mg of sucrose; about 0.78 mg of L-Histidine; about 0.02 mg polysorbate-80; about 0.25 mg aluminum; and further comprising 0.5 mL water, per dose.
C109. The method according to any one of clauses C94 to C97, wherein the composition is capable of eliciting a booster immune response to each of the N. meningitidis serogroups A, C, W-135 and Y.
C110. The method according to any one of clauses C94 to C97, wherein the composition is capable of eliciting a booster immune response to N. meningitidis serogroup B.
C111. The method according to any one of clauses C94 to C97, wherein the human is aged between 10 to 26 years old.
C112. The method according to any one of clauses C94 to C97, wherein the human is aged between 12 to <18 Months or 18 to <24 Months.
C113. The method according to any one of clauses C94 to C97, wherein the human is aged between 18 to <24 Months.
C114. The method according to any one of clauses C94 to C97, wherein the human is aged between ≥24 Months to <10 Years.
C115. The method according to any one of clauses C94 to C97, wherein the human is at least 16 years old.
C116. The method according to clause C115, comprising administering one dose to the human.
C117. The method according to any one of clauses C94 to C97, wherein the human is 10 to 12 years old.
C118. The method according to clause C113, comprising administering to the human at least two doses.
C119. The method according to clause C113, wherein the human is at most 16 years old.
C120. The method according to any one of clauses C94 to C97, comprising administering at least one dose of the composition to the human at about 11 years old and administering a further dose of the composition to the human at least four years after the first dose.
C121. The method according to any one of clauses C94 to C97, comprising administering at least one dose of the composition to the human at about 11 years old and administering a further dose of the composition to the human at least four years after the last dose.
C122. The method according to clause C116, wherein the further dose of the composition is administered about five years after the last dose.
C123. The method according to any one of clauses C94 to C97, comprising administering one dose of the composition to the human at about 11 years old and administering at least two doses of the composition to the human about five years after the first dose.
C124. The method according to any one of clauses C94 to C97, wherein the human is seronegative against N. meningitidis serogroups A, C, W-135 and Y capsular polysaccharides.
C125. The method according to any one of clauses C94 to C97, wherein the human is seropositive against N. meningitidis serogroups A, C, W-135 and Y capsular polysaccharides.
C126. The method according to any one of clauses C94 to C97, comprising administering a first dose and a second dose of the composition, wherein the second dose is about 6 months after the first dose.
C127. The method according to clause C122, wherein the human is at most 17 years of age.
C128. The method according to clause C122, comprising administering a third dose of the composition to the human at 16 years of age.
C129. The method according to any one of clauses C94 to C97, comprising administering at most two doses of the composition, wherein the second dose is about 6 months after the first dose.
C130. The method according to any one of clauses C94 to C97, wherein the composition elicits an immune response against a N. meningitidis serogroup B strain expressing A22.
C131. The method according to any one of clauses C94 to C97, wherein the composition elicits an immune response against a N. meningitidis serogroup B strain expressing A56.
C132. The method according to any one of clauses C94 to C97, wherein the composition elicits an immune response against a N. meningitidis serogroup B strain expressing B24.
C133. The method according to any one of clauses C94 to C97, wherein the composition elicits an immune response against a N. meningitidis serogroup B strain expressing B44.
C134. The method according to any one of clauses C94 to C97, wherein the composition induces a bactericidal immune response against any one of N. meningitidis serogroup B A22, A56, B24, B44 strains, or any combination thereof.
C135. The method according to any one of clauses C94 to C97, wherein the composition induces a bactericidal immune response against any one of N. meningitidis serogroup B B24, B16, B44, A22, B03, B09, A12, A19, A05, A07, B153 strains, or any combination thereof.

1-39. (canceled)

40. A method of using a kit to induce an immune response to N. meningitidis serogroups A, B, C, W-135 and Y in a human subject aged between 10 to 26 years old, comprising
preparing an immunogenic composition by reconstituting a lyophilized composition in a bivalent liquid composition, and
administering to the human subject a first dose and a second dose of the immunogenic composition, wherein the second dose is administered to the subject about 6 months after the first dose;
wherein the kit comprises:
a) the bivalent liquid composition comprises a first purified, lipidated polypeptide comprising the amino acid sequence set forth in SEQ ID NO: 1; a second purified, lipidated polypeptide comprising the amino acid sequence set forth in SEQ ID NO: 2; and aluminum; and
b) the lyophilized quadrivalent conjugate composition comprises:
(i) a purified Neisseria meningitidis serogroup A capsular polysaccharide (MenA) individually conjugated to an adipic acid dihydrazide (ADH) linker by 1-cyano-4-dimethylamino pyridinium tetrafluoroborate, wherein the linker is conjugated to tetanus toxoid (TT) by carbodiimide chemistry;
(ii) a purified Neisseria meningitidis serogroup C capsular polysaccharide (MenC) individually conjugated to an ADH linker by 1-cyano-4-dimethylamino pyridinium tetrafluoroborate, wherein the linker is conjugated to tetanus toxoid (TT) by carbodiimide chemistry;
(iii) a purified Neisseria meningitidis serogroup W capsular polysaccharide (MenW) individually conjugated directly to tetanus toxoid (TT) by 1-cyano-4-dimethylamino pyridinium tetrafluoroborate, in the absence of a linker; and
(iv) a purified Neisseria meningitidis serogroup Y capsular polysaccharide (MenY) individually conjugated directly to tetanus toxoid (TT) by 1-cyano-4-dimethylamino pyridinium tetrafluoroborate, in the absence of a linker;
wherein the human subject is naive to vaccination with N. meningitidis serogroups A, C, W-135 and Y capsular polysaccharides; and
wherein each dose comprises about 60 μg of the first polypeptide, about 60 μg of the second polypeptide, about 0.25 mg of aluminum, about 5 μg of the MenA capsular saccharide, about 5 μg of the MenC capsular saccharide, about 5 μg of the MenW capsular saccharide, and about 5 μg of the MenY capsular saccharide.

41. The method of claim 40, wherein the kit comprises the bivalent liquid composition contained within a pre-filled syringe (PFS).

42. The method of claim 41, wherein the kit comprises the lyophilized composition contained within a vial.

43. The method of claim 42, wherein the kit comprises a vial adapter.

44. The method of claim 43, wherein the immunogenic composition is prepared by affixing the vial adapter to the vial, attaching the PFS to the vial adapter, transferring the bivalent liquid composition into the vial, reconstituting the lyophilized composition with the bivalent liquid composition, and withdrawing the contents of the vial into the syringe via the vial adapter.

45. The method of claim 41, wherein the kit comprises the PFS stored in a tip cap up orientation.

46. The method of claim 41, wherein the kit comprises the PFS stored in a tip cap horizontal orientation.

47. The method of claim 41, wherein the first dose and the second are about 0.5 mL.

48. The method of claim 47, wherein the PFS has a fill volume between about 0.6 mL and about 0.7 mL.

49. The method of claim 40, wherein the kit comprises MenA polysaccharide conjugated to TT with a mean TT/polysaccharide ratio of 3.

50. The method of claim 40, wherein the kit comprises MenC polysaccharide conjugated to TT with a mean TT/polysaccharide ratio of 3.

51. The method of claim 40, wherein the kit comprises MenY polysaccharide conjugated to TT with a mean TT/polysaccharide ratio of 1.3.

52. The method of claim 40, wherein the kit comprises MenW polysaccharide conjugated to TT with a mean TT/polysaccharide ratio of 1.5.

53. The method of claim 40, wherein the aluminum is aluminum phosphate.

54. The method of claim 53, wherein the immunogenic composition comprises Tris-HCl, sodium chloride, sucrose, histidine, and polysorbate 80.

55. The method of claim 40, wherein the first polypeptide consists of the amino acid sequence set forth in SEQ ID NO: 1.

56. The method of claim 40, wherein the first polypeptide consists of the amino acid sequence set forth in SEQ ID NO: 2.

57. The method of claim 40, wherein between 90% and 100% of the amount of the first polypeptide is bound to the aluminum for 24 hours and between 90% and 100% of the amount of the second polypeptide is bound to the aluminum for 24 hours.

58. The method of claim 40, wherein the immunogenic composition does not comprise diphtheria toxoid or CRM.


SEQUENCE LISTING (74 sequences)

SEQ ID NO: 1 - protein, 258 aa, synthetic construct
  REGION 1..258: note = Synthetic amino acid sequence
CGSSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 2 - protein, 261 aa, synthetic construct
  REGION 1..261: note = Synthetic amino acid sequence
CGSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 3 - protein, 4 aa, synthetic construct
  REGION 1..4: note = Synthetic amino acid sequence
CGSS

SEQ ID NO: 4 - protein, 12 aa, synthetic construct
  REGION 1..12: note = Synthetic amino acid sequence
CGSSGGGGVAAD

SEQ ID NO: 5 - protein, 15 aa, synthetic construct
  REGION 1..15: note = Synthetic amino acid sequence
CSSGSGSGGGGVAAD

SEQ ID NO: 6 - protein, 255 aa, synthetic construct
  REGION 1..255: note = Synthetic amino acid sequence
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVDLAAAYIKPDEKHHAVISGSVLYNQDEKGSYSLGIFGGKAEEVAGSAEVKTVNGIRHIGLAAKQ

SEQ ID NO: 7 - protein, 259 aa, Neisseria meningitidis
  source 1..259: note = Group B
CSSGGGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGDKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 8 - protein, 261 aa, Neisseria meningitidis
  source 1..261: note = Group B
CSSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 9 - protein, 254 aa, Neisseria meningitidis
  source 1..254: note = Group B
CSSGGGGVAADIGAGLADALTAPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 10 - protein, 254 aa, Neisseria meningitidis
  source 1..254: note = Group B
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 11 - protein, 263 aa, Neisseria meningitidis
  source 1..263: note = Group B
CSSGGGGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFDKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 12 - protein, 255 aa, Neisseria meningitidis
  source 1..255: note = Group B
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQEQDPEHSGKMVAKRRFKIGDIAGEHTSFDKLPKDVMATYRGTAFGSDDAGGKLTYTIDFAAKQGHGKIEHLKSPELNVELATAYIKPDEKHHAVISGSVLYNQDEKGSYSLGIFGGQAQEVAGSAEVETANGIHHIGLAAKQ

SEQ ID NO: 13 - protein, 255 aa, Neisseria meningitidis
  source 1..255: note = Group B
CSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 14 - protein, 255 aa, Neisseria meningitidis
  source 1..255: note = Group B
CSSGGGGVAADIGAVLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 15 - protein, 255 aa, Neisseria meningitidis
  source 1..255: note = Group B
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 16 - protein, 263 aa, Neisseria meningitidis
  source 1..263: note = Group B
CSSGGGGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 17 - protein, 255 aa, Neisseria meningitidis
  source 1..255: note = Group B
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 18 - protein, 254 aa, Neisseria meningitidis
  source 1..254: note = Group B
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPGGKAEYHGKAFSSDDPNGRLHYSIDFTKKQGYGRIEHLKTPEQNVELASAELKADEKSHAVILGDTRYGSEEKGTYHLALFGDRAQEIAGSATVKIREKVHEIGIAGKQ

SEQ ID NO: 19 - skipped

SEQ ID NO: 20 - protein, 257 aa, Neisseria meningitidis
  source 1..257: note = Group B
CSSGGGGVAADIGAGLADALTAPLDHKDKGLKSLTLEDSISQNGTLTLSAQGAEKTFKVGDKDNSLNTGKLKNDKISRFDFVQKIEVDGQTITLASGEFQIYKQNHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEISIAGKQ

SEQ ID NO: 21 - protein, 254 aa, Neisseria meningitidis
  source 1..254: note = Group B
CSSGGGGVAADIGAGLADALTTPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 22 - protein, 262 aa, Neisseria meningitidis
  source 1..262: note = Group B
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKIDSLINQRSFLVSGLGGEHTAFNQLPGDKAEYHGKAFSSDDPNGRLHYTIDFTNKQGYGRIEHLKTPELNVDLASAELKADEKSHAVILGDTRYGSEEKGTYHLALFGDRAQEIAGSATVKIGEKVHEIGIAGKQ

SEQ ID NO: 23 - protein, 255 aa, Neisseria meningitidis
  source 1..255: note = Group B
CSSGGGGVAADIGAGLADALTAPLDHKDKGLQSLILDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFSSDDAGGKLIYTIDFAAKQGHGKIEHLKSPELNVDLAAADIKPDEKHHAVISGSVLYNQAEKGSYSLGIFGGKAQEVAGSAEVKTVNGIRHIGLAAKQ

SEQ ID NO: 24 - protein, 17 aa, synthetic construct
  REGION 1..17: note = Synthetic amino acid sequence
CGSSGGGGSGGGGVTAD

SEQ ID NO: 25 - protein, 16 aa, synthetic construct
  REGION 1..16: note = Synthetic amino acid sequence
CSSGGGGSGGGGVTAD

SEQ ID NO: 26 - protein, 255 aa, Neisseria meningitidis; identical to SEQ ID NO: 17

SEQ ID NO: 27 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 28 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 29 - protein, 260 aa, Neisseria meningitidis
CSSGGGGSGGGGVAADIGTGLADALTAPLDHKDKGLQSLMLDQSVRKNEKLKLSAQGAEKTYGNGDSLNTGKLKNDKISRFDFIHQIEVDGQLITLESGEFQVYKQSHSALTALQTEQVQDSEHSEKMVAKRRFKIGDIAGEHTSFDKLPKDVMATYRGTAFGSDDAGGKLTYTIDFAAKQGHGKIEHLKSPELNVELAAAYIKPDEKRHAVISGSVLYNQDEKGSYSLGIFGGQAQEVAGSAEVETANGIHHIGLAAKQ

SEQ ID NO: 30 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTALQTEQEQDPEHSGKMVAKRRFKIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVKTANGIHHIGLAAKQ

SEQ ID NO: 31 - protein, 254 aa, synthetic construct
  REGION 1..254: note = Synthetic amino acid sequence
SSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 32 - protein, 253 aa, synthetic construct
  REGION 1..253: note = Synthetic amino acid sequence
SSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPGGKAEYHGKAFSSDDPNGRLHYSIDFTKKQGYGRIEHLKTPEQNVELASAELKADEKSHAVILGDTRYGSEEKGTYHLALFGDRAQEIAGSATVKIREKVHEIGIAGKQ

SEQ ID NO: 33 - protein, 253 aa, synthetic construct
  REGION 1..253: note = Synthetic amino acid sequence
SSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 34 - protein, 256 aa, synthetic construct
  REGION 1..256: note = Synthetic amino acid sequence
SSGGGGVAADIGAGLADALTAPLDHKDKGLKSLTLEDSISQNGTLTLSAQGAEKTFKVGDKDNSLNTGKLKNDKISRFDFVQKIEVDGQTITLASGEFQIYKQNHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEISIAGKQ

SEQ ID NO: 35 - protein, 253 aa, synthetic construct
  REGION 1..253: note = Synthetic amino acid sequence
SSGGGGVAADIGAGLADALTTPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 36 - protein, 261 aa, synthetic construct
  REGION 1..261: note = Synthetic amino acid sequence
SSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKIDSLINQRSFLVSGLGGEHTAFNQLPGDKAEYHGKAFSSDDPNGRLHYTIDFTNKQGYGRIEHLKTPELNVDLASAELKADEKSHAVILGDTRYGSEEKGTYHLALFGDRAQEIAGSATVKIGEKVHEIGIAGKQ

SEQ ID NO: 37 - protein, 254 aa, synthetic construct
  REGION 1..254: note = Synthetic amino acid sequence
SSGGGGVAADIGAGLADALTAPLDHKDKGLQSLILDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFSSDDAGGKLIYTIDFAAKQGHGKIEHLKSPELNVDLAAADIKPDEKHHAVISGSVLYNQAEKGSYSLGIFGGKAQEVAGSAEVKTVNGIRHIGLAAKQ

SEQ ID NO: 38 - protein, 254 aa, synthetic construct; identical to SEQ ID NO: 31
  REGION 1..254: note = Synthetic amino acid sequence

SEQ ID NO: 39 - protein, 253 aa, synthetic construct
  REGION 1..253: note = Synthetic amino acid sequence
SSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 40 - protein, 253 aa, synthetic construct; identical to SEQ ID NO: 33
  REGION 1..253: note = Synthetic amino acid sequence

SEQ ID NO: 41 - protein, 259 aa, synthetic construct
  REGION 1..259: note = Synthetic amino acid sequence
SSGGGGSGGGGVAADIGTGLADALTAPLDHKDKGLQSLMLDQSVRKNEKLKLSAQGAEKTYGNGDSLNTGKLKNDKISRFDFIHQIEVDGQLITLESGEFQVYKQSHSALTALQTEQVQDSEHSEKMVAKRRFKIGDIAGEHTSFDKLPKDVMATYRGTAFGSDDAGGKLTYTIDFAAKQGHGKIEHLKSPELNVELAAAYIKPDEKRHAVISGSVLYNQDEKGSYSLGIFGGQAQEVAGSAEVETANGIHHIGLAAKQ

SEQ ID NO: 42 - protein, 254 aa, synthetic construct
  REGION 1..254: note = Synthetic amino acid sequence
SSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTALQTEQEQDPEHSGKMVAKRRFKIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVKTANGIHHIGLAAKQ

SEQ ID NO: 43 - protein, 262 aa, synthetic construct
  REGION 1..262: note = Synthetic amino acid sequence
SSGGGGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 44 - protein, 254 aa, synthetic construct
  REGION 1..254: note = Synthetic amino acid sequence
SSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 45 - protein, 259 aa, synthetic construct
  REGION 1..259: note = Synthetic amino acid sequence
SSGGGGSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 46 - protein, 260 aa, synthetic construct
  REGION 1..260: note = Synthetic amino acid sequence
SSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 47 - protein, 259 aa, synthetic construct
  REGION 1..259: note = Synthetic amino acid sequence
SSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 48 - protein, 260 aa, synthetic construct
  REGION 1..260: note = Synthetic amino acid sequence
CSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 49 - protein, 255 aa, synthetic construct; identical to SEQ ID NO: 23
  REGION 1..255: note = Synthetic amino acid sequence

SEQ ID NO: 50 - protein, 255 aa, synthetic construct; identical to SEQ ID NO: 17
  REGION 1..255: note = Synthetic amino acid sequence

SEQ ID NO: 51 - protein, 255 aa, synthetic construct
  REGION 1..255: note = Synthetic amino acid sequence
GSSGGGGVAADIGAVLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 52 - protein, 254 aa, synthetic construct
  REGION 1..254: note = Synthetic amino acid sequence
GSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 53 - protein, 258 aa, synthetic construct
  REGION 1..258: note = Synthetic amino acid sequence
SSGGGGSGGGGVAADIGAGLADALTAPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 54 - protein, 253 aa, synthetic construct
  REGION 1..253: note = Synthetic amino acid sequence
SSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 55 - protein, 255 aa, synthetic construct
  REGION 1..255: note = Synthetic amino acid sequence
CSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 56 - protein, 254 aa, synthetic construct
  REGION 1..254: note = Synthetic amino acid sequence
SSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 57 - protein, 262 aa, synthetic construct; identical to SEQ ID NO: 22
  REGION 1..262: note = Synthetic amino acid sequence

SEQ ID NO: 58 - protein, 254 aa, synthetic construct
  REGION 1..254: note = Synthetic amino acid sequence
SSGGGGVAADIGAVLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 59 - protein, 258 aa, synthetic construct; identical to SEQ ID NO: 1
  REGION 1..258: note = Synthetic amino acid sequence

SEQ ID NO: 60 - protein, 257 aa, synthetic construct
  REGION 1..257: note = Synthetic amino acid sequence
GSSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 61 - protein, 254 aa, synthetic construct
  REGION 1..254: note = Synthetic amino acid sequence
SSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 62 - protein, 252 aa, synthetic construct
  REGION 1..252: note = Synthetic amino acid sequence
GGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 63 - protein, 262 aa, Neisseria meningitidis
  source 1..262: note = Group B
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGGKAEYHGKAFSSDDPNGRLHYSIDFTKKQGYGGIEHLKTPEQNVELASAELKADEKSHAVILGDTRYGSEEKGTYHLALFGDRAQEIAGSATVKIGEKVHEIGIAGKQ

SEQ ID NO: 64 - protein, 262 aa, Neisseria meningitidis
  source 1..262: note = Group B
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEISIAGKQ

SEQ ID NO: 65 - protein, 257 aa, Neisseria meningitidis
  source 1..257: note = Group B
CSSGGGGVAADIGAGLADALTAPLDHKDKGLKSLTLEDSISQNGTLTLSAQGAERTFKAGNKDNSLNTGKLKNDKISRFDFVQKIEVDGQTITLASGEFQIYKQNHSAVVALQIEKINNPDKIDSLINQRSFLVSSLGGEHTAFNQLPGGKAEYHGKAFSSDDPNGRLHYSIDFTKKQGYGRIEHLKTPEQNVELAAAELKADEKSHAVILGDTRYGSEEKGTYHLALFGDRAQEIAGSATVKIGEKVHEIGIAGKQ

SEQ ID NO: 66 - protein, 262 aa, Neisseria meningitidis
  source 1..262: note = Group B
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYDSEEKGTYHLALFGDRAQEIAGSATVKIGEKVHEISIAGKQ

SEQ ID NO: 67 - protein, 262 aa, Neisseria meningitidis
  source 1..262: note = Group B
CSSGGGGSGGGGVAADIGAGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQTEKVNNPDKTDSLINQRSFLVSGLGGEHTAFNQLPVGKSEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEISIAGKQ

SEQ ID NO: 68 - protein, 255 aa, Neisseria meningitidis
  source 1..255: note = Group B
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTALQTEQVQDSEDSGKMVAKRQFRIGDIAGEHTSFDKLPKGGSATYRGTAFSSDDAGGKLTYTIDFAAKQGHGKIEHLKSPELNVELATAYIKPDEKRHAVISGSVLYNQDEKGSYSLGIFGGQAQEVAGSAEVETANGIQHIGLAAKQ

SEQ ID NO: 69 - protein, 255 aa, Neisseria meningitidis
  source 1..255: note = Group B
CSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTALGSDDAGGKLTYTIDFAAKQGHGKIEHLKSPELNVELATAYIKPDEKRHAVISGSVLYNQDEKGSYSLGIFGGQAQEVAGSAEVETVNGIHHIGLAAKQ

SEQ ID NO: 70 - protein, 255 aa, Neisseria meningitidis
  source 1..255: note = Group B
CSSGGGGVAADIGAGLADALTAPLDHKDKSLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 71 - protein, 255 aa, Neisseria meningitidis
  source 1..255: note = Group B
CSSGGGGVAADIGAGLADALTAPLDHKDKGLQSLTLDQSVRKNEKLKLAAQGAEKTYGNGDSLNTGKLKNDKVSRFDFIRQIEVDRQLITLESGEFQVYKQSHSALTALQTEQVQDSEHSGKMVAKRQFRIGDIAGEHTSFDKLPEGGRATYRGTAFGSDDAGGKLIYTIDFAAKQGHGKIEHLKSPELNVDLAAADIKPDEKHHAVISGSVLYNQAEKGSYSLGIFGGKAQEVAGSAEVKTVNGIRHIGLAAKQ

SEQ ID NO: 72 - protein, 255 aa, Neisseria meningitidis
  source 1..255: note = Group B
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTALQTEQEQDLEHSGKMVAKRRFKIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVKTANGIHHIGLAAKQ

SEQ ID NO: 73 - protein, 258 aa, Neisseria meningitidis
  source 1..258: note = Group B
CSSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 74 - protein, 257 aa, Neisseria meningitidis
  source 1..257: note = Group B
CSSGGGGVAADIGAGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ